(19) United States
(12) Patent Application Publication
(10) Pub. No.: US 2024/0417788 A1
(43) Pub. Date: Dec. 19, 2024

(54) SPATIALLY RESOLVED EPIGENOME-TRANSCRIPTOME CO-PROFILING

(71) Applicant: Yale University, New Haven, CT (US)

(72) Inventors: Rong Fan, Cheshire, CT (US); Di Zhang, New Haven, CT (US); Yanxiang Deng, New Haven, CT (US)

(73) Assignee: Yale University, New Haven, CT (US)

(21) Appl. No.: 18/740,662

(22) Filed: Jun. 12, 2024

Related U.S. Application Data
(60) Provisional application No. 63/507,841, filed on Jun. 13, 2023.

(51) Int. Cl. C12Q 1/6855; B01L 3/00; C12Q 1/6804; C12Q 1/6806; C12Q 1/6869; G01N 1/30

(52) U.S. Cl. CPC C12Q 1/6855; B01L 3/502761; C12Q 1/6804; C12Q 1/6806; C12Q 1/6869; G01N 1/30; B01L 2300/0609; B01L 2300/0883; B01L 2300/0893; G01N 2001/305

(57) ABSTRACT

Provided herein are compositions and methods for high resolution spatial transcriptomic and epigenomic co-profiling of a biological sample.

### STATEMENT REGARDING FEDERALLY SPONSORED RESEARCH OR DEVELOPMENT

This invention was made with government support under U54AG076043, UG3CA257393, ROICA245313, and RF1MH128876 awarded by the National Institutes of Health (NIH). The government has certain rights in the invention.

### REFERENCE TO A “SEQUENCE LISTING,” SUBMITTED AS AN XML FILE

The Sequence Listing written in the xml file: “046172-5333-P1US_SequenceListing.xml”; created on Jun. 12, 2023, and 196,497 bytes in size, is hereby incorporated by reference.

### BACKGROUND OF THE INVENTION

Single-cell multi-omics allows for uncovering the mechanism of gene regulation across different omics layers (Allaway, 2021, Nature, 597:693-697; Chen, 2019, Nat Biotechnol, 37:1452-1457; Cao, 2018, Science, 361:1380-1385; Trevino, 2021, Cell, 184:5053-5069) but lacks spatial information, which is crucial to understanding cellular function in tissue. Recently, spatial epigenomics, transcriptomics, and proteomics emerged (Liu, 2021, Cell, 183:1665-1681; Deng, 2022, Science, 375:681-686; Deng, 2022, Nature, 609:375-383; Chen, 2022, Cell, 185:1777-1792; Lu, 2022, Cell, 185:4448-4464) but most of them can capture only one layer of the omics information. Although computational methods can integrate data from multiple omics (Ma, 2020, Cell, 183:1103-1116), they cannot readily uncover the mechanistic link between different omics layers.

Spatial omics technologies (spatial epigenomics, transcriptomics, and proteomics) based on either next-generation sequencing (NGS) (Liu, 2021, Cell, 183:1665-1681; Deng, 2022, Science, 375:681-686; Deng, 2022, Nature, 609:375-383; Chen, 2022, Cell, 185:1777-1792; Thornton, 2021, Nature Communications, 12:1274; Cho, 2021, Cell, 184:3559-3572; Fu, 2022, Cell, 185:4621-4633) or imaging (Lu, 2022, Cell, 185:4448-4464; Fang, 2022, Science, 377:52-62) offer an unprecedented opportunity to generate new and rich insights into gene regulation in the spatial tissue context. However, in order to comprehensively understand the mechanism of gene regulation, different layers of omics information need to be profiled simultaneously. It was first demonstrated in dissociated single cells (Allaway, 2021, Nature, 597:693-697; Chen, 2019, Nat Biotechnol, 37:1452-1457; Cao, 2018, Science, 361:1380-1385; Trevino, 2021, Cell, 184:5053-5069), but yet to be realized directly in tissue. Imaging-based DNA seqFISH+ combined with RNA seqFISH detected spatial chromatin and gene expression for target genes and genomic loci (Takei, 2021, Nature, 590:344-350). As of today, current technologies have not been able to perform unbiased genome-wide co-mapping of epigenome and transcriptome on the same tissue section at cellular level.

Thus, there is a need in the art for improved compositions and methods for performing unbiased genome-wide co-mapping of epigenome and transcriptome on the same tissue section at cellular level. This invention satisfies this unmet need.

### SUMMARY OF THE INVENTION

In one embodiment, the invention relates to a method comprising the steps of:
(a) delivering to a region of interest in a tissue sample mounted on a substrate reagents for transposition including a Tn5 transposition complex pre-loaded with a DNA adapter containing a universal ligation linker and reagents for reverse transcription including a DNA adapter containing a universal ligation linker and an RNA detection probe;
(b) delivering to the region of interest a first set of barcoded polynucleotides, wherein the barcoded polynucleotides comprise a first region for ligation to the linker adaptor sequence, a second unique region for spatial barcoding and a third linker region for ligation to a region of the second barcode or a universal ligation linker, wherein the first set of barcoded polynucleotides is delivered through a first microfluidic device clamped to the region of interest;
(c) delivering to the region of interest ligation reagents to join the ligation adaptor to the barcoded polynucleotides of the first set;
(d) delivering to the region of interest a second set of barcoded polynucleotides, wherein the barcoded polynucleotides comprise a first region for ligation to the linker region of the first barcode or a universal ligation linker, a second unique region for spatial barcoding and a third ligation region comprising a sequence for recognition by a primer for DNA amplification, wherein the second set of barcoded polynucleotides is delivered through a second microfluidic device clamped to the region of interest, wherein the second microfluidic device is oriented on the region of interest perpendicular to the direction of the microchannels of the first microfluidic device;
(e) delivering to the region of interest ligation reagents to join barcoded polynucleotides of the first set to barcoded polynucleotides of the second set;
(f) imaging the region of interest to produce a sample image;
(g) delivering to the region of interest lysis buffer or denaturation reagents to produce a lysed or denatured tissue sample; and
(h) extracting the cDNA and genomic DNA from the lysed or denatured tissue sample.

In one embodiment, the RNA detection probe comprises a poly-T sequence that binds to the poly-A trail of mRNAs.

In one embodiment, the method further comprises a step of permeabilizing the tissue sample prior to delivering the transposase and linker adaptor sequence.

In one embodiment, the method further comprises delivering to the biological sample a ligation linker sequence, wherein the ligation linker is selected from:
a) a nucleic acid molecule comprising a sequence complementary to the ligation linker sequence of the ligation adaptor associated with the transposon and a sequence complementary to the ligation linker sequence of the barcoded polynucleotides of the first set; or
b) a nucleic acid molecule comprising a sequence complementary to the ligation linker sequence of the barcoded polynucleotides of the first set and a sequence complementary to the ligation linker sequence of the barcoded polynucleotides of the second set.

In one embodiment, the method further comprises step (i) sequencing the cDNA, the genomic DNA or a combination thereof.

In one embodiment, the method further comprises constructing at least one of a spatial transcriptomic map and a spatial epigenomic map of the tissue section by matching the spatially addressable barcoded conjugates to corresponding sequencing reads.

In one embodiment, the method further comprises identifying the anatomical location of the nucleic acids by correlating the spatial map to the sample image.

In one embodiment, the tissue section mounted on a slide is produced by:
sectioning a formalin fixed paraffin embedded (FFPE) tissue, optionally into a 5-10 μm section and mounting the tissue section onto a substrate, optionally a poly-L-lysine-coated slide;
applying to the tissue section a wash solution, optionally a xylene solution, to deparaffinize the tissue section;
applying to the tissue section a rehydration solution to rehydrate the tissue section;
applying to the tissue section an enzymatic solution to permeabilize the tissue section; and
applying formalin to the tissue section to post-fix the tissue section.

In one embodiment, the first and/or second microfluidic device is fabricated from polydimethylsiloxane (PDMS).

In one embodiment, the first and/or second microfluidic device comprises 10 to 1000 microchannels.

In one embodiment, the first and/or second microfluidic device comprises serpentine microchannels.

In one embodiment, the method further comprises delivering to the region of interest a third set of barcoded polynucleotides, wherein the third set of barcoded polynucleotides is delivered to specific zones, such that each zone distinguishes a specific region of overlap of the first and second barcode sequences; wherein the third set of barcoded polynucleotides are delivered directly to the tissue section, optionally through a set of holes in a device clamped to the substrate, wherein each hole is positioned directly above a zone of overlap of the first and second barcode sequences.

In one embodiment, delivery of the first set of barcoded polynucleotides is delivered through the first microfluidic device using a negative pressure system and/or delivery of the second set of barcoded polynucleotides is delivered through the second microfluidic device using a negative pressure system.

In one embodiment, the lysis buffer or denaturation reagents are delivered directly to the tissue section, optionally through a hole in a device clamped to the substrate, wherein the hole is positioned directly above the region of interest.

In one embodiment, the first set of barcoded polynucleotides comprises SEQ ID NO:1-100. In one embodiment, the second set of barcoded polynucleotides comprises SEQ ID NO:101-200.

In one embodiment, the imaging is with an optical or fluorescence microscope.

In one embodiment, the substrate is a glass slide or a plastic slide.

In one embodiment, the invention relates to a method comprising the steps of:
(a) delivering to a region of interest in a tissue sample mounted on a substrate reagents for spatial tagmentation including (i) a primary antibody specific for binding to an epigenomic marker of interest (ii) a secondary antibody and (iii) protein A tethered Tn5-DNA complex pre-loaded with a DNA adapter containing a universal ligation linker, and reagents for reverse transcription including a DNA adapter containing a universal ligation linker and an RNA detection probe;
(b) delivering to the region of interest a first set of barcoded polynucleotides, wherein the barcoded polynucleotides comprise a first region for ligation to the linker adaptor sequence, a second unique region for spatial barcoding and a third linker region for ligation to a region of the second barcode or a universal ligation linker, wherein the first set of barcoded polynucleotides is delivered through a first microfluidic device clamped to the region of interest;
(c) delivering to the region of interest ligation reagents to join the ligation adaptor to the barcoded polynucleotides of the first set;
(d) delivering to the region of interest a second set of barcoded polynucleotides, wherein the barcoded polynucleotides comprise a first region for ligation to the linker region of the first barcode or a universal ligation linker, a second unique region for spatial barcoding and a third ligation region comprising a sequence for recognition by a primer for DNA amplification, wherein the second set of barcoded polynucleotides is delivered through a second microfluidic device clamped to the region of interest, wherein the second microfluidic device is oriented on the region of interest perpendicular to the direction of the microchannels of the first microfluidic device;
(e) delivering to the region of interest ligation reagents to join barcoded polynucleotides of the first set to barcoded polynucleotides of the second set;
(f) imaging the region of interest to produce a sample image;
(g) delivering to the region of interest lysis buffer or denaturation reagents to produce a lysed or denatured tissue sample; and
(h) extracting the cDNA and genomic DNA from the lysed or denatured tissue sample.

In one embodiment, the method further comprises a step of permeabilizing the tissue sample prior to delivering the transposase and linker adaptor sequence.

In one embodiment, the primary antibody is a whole antibody, a Fab antibody fragment, a F(ab′)₂ antibody fragment, a monospecific Fab₂ fragment, a bispecific Fab₂ fragment, a trispecific Fab₃ fragment, a single chain variable fragment (scFv), a bispecific diabody, a trispecific diabody, an scFv-Fc molecule, a nanobody, or a minibody.

In one embodiment, the epigenomic marker is H2AK5ac, H2AK9ac, H2BK120ac, H2BK12ac, H2BK15ac, H2BK20ac, H2BK5ac, H2Bub, H3, H3ac, H3K14ac, H3K18ac, H3K23ac, H3K23me2, H3K27me1, H3K27me2, H3K36ac, H3K36me1, H3K36me2, H3K4ac, H3K56ac, H3K79me1, H3K79me3, H3K9acS10ph, H3K9me2, H3S10ph, H3T11ph, H4, H4ac, H4K12ac, H4K16ac, H4K5ac, H4K8ac, H4K91ac, H3F3A, H3K27me3, H3K36me3, H3K4me1, H3K79me2, H3K9me1, H3K9me2, H3K9me3, H4K20me1, H2AFZ, H3K27ac, H3K4me2, H3K4me3, or H3K9ac, or any combination thereof.

In one embodiment, the RNA detection probe comprises a poly-T sequence that binds to the poly-A trail of mRNAs.

In one embodiment, the method further comprises delivering to the biological sample a ligation linker sequence, wherein the ligation linker is selected from:
a) a nucleic acid molecule comprising a sequence complementary to the ligation linker sequence of the ligation adaptor associated with the transposon and a sequence complementary to the ligation linker sequence of the barcoded polynucleotides of the first set; or
b) a nucleic acid molecule comprising a sequence complementary to the ligation linker sequence of the barcoded polynucleotides of the first set and a sequence complementary to the ligation linker sequence of the barcoded polynucleotides of the second set.

In one embodiment, the method further comprises step (i) sequencing the cDNA, the genomic DNA or a combination thereof.

In one embodiment, the method further comprises constructing at least one of a spatial transcriptomic map and a spatial epigenomic map of the tissue section by matching the spatially addressable barcoded conjugates to corresponding sequencing reads.

In one embodiment, the method further comprises identifying the anatomical location of the nucleic acids by correlating the spatial map to the sample image.

In one embodiment, the tissue section mounted on a slide is produced by:
sectioning a fixed frozen tissue or a formalin fixed paraffin embedded (FFPE) tissue, optionally into a 5-10 μm section and mounting the tissue section onto a substrate, optionally a poly-L-lysine-coated slide;
applying to the tissue section a wash solution, optionally a xylene solution, to deparaffinize the tissue section;
applying to the tissue section a rehydration solution to rehydrate the tissue section;
applying to the tissue section an enzymatic solution to permeabilize the tissue section; and
applying formalin to the tissue section to post-fix the tissue section.

In one embodiment, the first and/or second microfluidic device is fabricated from polydimethylsiloxane (PDMS), rubber, plastic or glass.

In one embodiment, the first and/or second microfluidic device comprises 10 to 1000 microchannels.

In one embodiment, the first and/or second microfluidic device comprises serpentine microchannels.

In one embodiment, the method further comprises delivering to the region of interest a third set of barcoded polynucleotides, wherein the third set of barcoded polynucleotides is delivered to specific zones, such that each zone distinguishes a specific region of overlap of the first and second barcode sequences; wherein the third set of barcoded polynucleotides are delivered directly to the tissue section, optionally through a set of holes in a device clamped to the substrate, wherein each hole is positioned directly above a zone of overlap of the first and second barcode sequences.

In one embodiment, delivery of the first set of barcoded polynucleotides is delivered through the first microfluidic device using a negative pressure system and/or delivery of the second set of barcoded polynucleotides is delivered through the second microfluidic device using a negative pressure system.

In one embodiment, the lysis buffer or denaturation reagents are delivered directly to the tissue section, optionally through a hole in a device clamped to the substrate, wherein the hole is positioned directly above the region of interest.

In one embodiment, the first set of barcoded polynucleotides comprises SEQ ID NO:1-100. In one embodiment, the second set of barcoded polynucleotides comprises SEQ ID NO:101-200.

In one embodiment, the imaging is with an optical, fluorescence or Raman microscope.

In one embodiment, the substrate is a glass slide, a plastic slide, a silicon wafer, a silica wafter, or other solid support surface.

In one embodiment, the invention relates to set of polynucleotides for spatially barcoding a sequencing library. In one embodiment, the set of barcoded polynucleotides comprising SEQ ID NO:1-100. In one embodiment, the set of barcoded polynucleotides comprises SEQ ID NO:101-200. In one embodiment, the set of barcoded polynucleotides comprises SEQ ID NO:1-200.

In one embodiment, the invention relates to a kit comprising one or more set of barcoded polynucleotides for spatially barcoding a sequencing library. In one embodiment, the kit comprises a set of barcoded polynucleotides comprising SEQ ID NO: 1-100. In one embodiment, the kit comprises a set of barcoded polynucleotides comprises SEQ ID NO:101-200. In one embodiment, the kit comprises a first set of barcoded polynucleotides comprising SEQ ID NO: 1-100, and a second set of barcoded polynucleotides comprises SEQ ID NO:101-200.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1H depict the design and evaluation of spatial epigenome-transcriptome co-sequencing with E13 mouse embryo. (FIG. 1A) Schematic workflow. (FIG. 1B) Comparison of number of unique fragments and fraction of reads in peaks (FRP) in spatial-ATAC-RNA-seq and spatial-CUT&Tag-RNA-seq. (FIG. 1C) Gene and UMI (unique molecular identifier) count distribution in spatial-ATAC-RNA-seq and spatial-CUT&Tag-RNA-seq. The number of pixels in E13: 2,187; Human brain: 2,500; Mouse brain (ATAC): 9,215; Mouse brain (H3K27me3): 9,752; Mouse brain (H3K27ac): 9,370; Mouse brain (H3K4me3): 9,548. The box plots show the median (centre line), the first and third quartiles (box limits), and 1.5× the interquartile range (whiskers). (FIG. 1D) Spatial distribution and UMAP of all the clusters for ATAC, RNA, and joint clustering of ATAC and RNA data. Overlay of clusters with the tissue image reveals that the spatial clusters precisely match the anatomic regions. Pixel size, 50 μm. Scale bar, 1 mm. (FIG. 1E) Spatial mapping of GAS and gene expression for selected marker genes in different clusters for ATAC and RNA in spatial-ATAC-RNA-seq. (FIG. 1F) Pseudotime analysis from radial glia to postmitotic premature neurons visualized at a spatial level. (FIG. 1G) Heatmaps delineating gene expression and gene activity scores for marker genes. (FIG. 1H) Dynamic changes of gene activity scores and gene expression across pseudotime.

FIG. 2A through FIG. 2E depict spatial chromatin accessibility and transcriptome co-profiling of P22 mouse brain. (FIG. 2A) Design of the microfluidic chips for 100×100 barcodes with 20 μm channel size. (FIG. 2B) Spatial distribution and UMAP of all the clusters for ATAC and RNA in spatial-ATAC-RNA-seq for the mouse brain. Pixel size, 20 μm. Scale bar, 1 mm. (FIG. 2C) Integration of ATAC data with scATAC-seq data (Li, 2021, Nature 598:129-136) from mouse brain. (FIG. 2D) Integration of RNA data with scRNA-seq (Zeisel, 2018, Cell, 174:999-1014) from mouse brain. (FIG. 2E) Spatial mapping of GAS and gene expression for selected marker genes in different clusters for ATAC and RNA in spatial-ATAC-RNA-seq.

FIG. 3A through FIG. 3F depict spatial histone modification and transcriptome co-profiling of P22 mouse brain. FIG. 3A to FIG. 3C depict spatial distribution and UMAP of all the clusters for H3K27me3 and RNA (FIG. 3A), H3K27ac and RNA (FIG. 3B), H3K4me3 and RNA (FIG. 3C) in the mouse brain. Pixel size, 20 μm. Scale bar, 1 mm. (FIG. 3D) Integration of H3K27me3 data with scCUT&Tag (H3K27me3) data (Bartosovic, 2022, Nat Biotechnol, doi: 10.1038/s41587-022-01535-4. Online ahead of print) from mouse brain. (FIG. 3E) Integration of H3K27ac data with scCUT&Tag (H3K27ac) data (Bartosovic, 2022, Nat Biotechnol, doi: 10.1038/s41587-022-01535-4. Online ahead of print) from mouse brain. (FIG. 3F) Integration of RNA data in spatial-CUT&Tag (H3K27me3)-RNA-seq, spatial-CUT&Tag (H3K27ac)-RNA-seq, and spatial-CUT&Tag (H3K4me3)-RNA-seq with scRNA-seq data (Zeisel, 2018, Cell, 174:999-1014) from mouse brain.

FIG. 4A through FIG. 4G depict region specific epigenetic regulation of gene expression. FIG. 4A to FIG. 4C depict correlation of H3K27me3 CSS and RNA gene expression: (FIG. 4A) H3K27ac GAS and RNA gene expression and (FIG. 4B) H3K4me3 GAS and RNA gene expression in (FIG. 4C) corpus callosum. (FIG. 4D) Upset plot of H3K27me3 CSS and RNA gene expression in striatum, deeper and superficial cortical layer. Low CSS or gene expression (−), High CSS or gene expression (+). (FIG. 4E-FIG. 4G) Venn diagrams showing the number of high (+) or low (−) CSS/GAS for different histone modifications in corpus callosum with common RNA marker genes.

FIG. 5A through FIG. 5D depict spatial chromatin accessibility and transcriptome co-profiling of human hippocampus. (FIG. 5A) Bright-field image, spatial distribution and UMAP of all the clusters based on ATAC and RNA in the human hippocampus. ML: molecular layer. Pyr: pyramidal neuron. Pixel size, 50 μm. Scale bar, 1 mm. (FIG. 5B) Integration of the ATAC data with scATAC-seq (Corces, M. R. et al., 2020, Nat Genet, 52:1158-1168) from human hippocampus. (FIG. 5C) Integration of the RNA data with snRNA-seq data from human brain (Franjic, D. et al., 2022, Neuron, 110:452-469). (FIG. 5D) Spatial mapping of GAS and gene expression for selected marker genes in different clusters for ATAC and RNA.

FIG. 6A through FIG. 6E depict the workflow of spatial-ATAC-RNA-seq and spatial-CUT&Tag-RNA-seq. (FIG. 6A) Schematic workflow of spatial-ATAC-RNA-seq and spatial-CUT&Tag-RNA-seq. (FIG. 6B-FIG. 6C) Chemistry workflow of ATAC (FIG. 6B) and RNA (FIG. 6C) in spatial-ATAC-RNA-seq. (FIG. 6D-FIG. 6E), Chemistry workflow of CUT&Tag (FIG. 6D) and RNA (FIG. 6E) in spatial-CUT&Tag-RNA-seq.

FIG. 7A through FIG. 7J depict further analysis of spatial-ATAC-RNA-seq for E13 mouse embryo. (FIG. 7A) H&E image from an adjacent tissue section of E13 mouse embryo. (FIG. 7B) Spatial mapping of GAS and gene expression for selected marker genes in spatial-ATAC-RNA-seq. (FIG. 7C) Integration of scRNA-seq data (Cao, 2019, Nature, 566:496-502) from E13.5 mouse embryos with ATAC and RNA data in spatial ATAC-RNA-seq. MOCA, Mouse Organogenesis Cell Atlas. (FIG. 7D) Spatial mapping of cell types identified by label transfer from scRNA-seq (Cao, 2019, Nature, 566:496-502) to ATAC (top) and RNA (bottom). (FIG. 7E) Genome track visualization of marker genes with peak-to-gene links for distal regulatory elements and peak co-accessibility. (FIG. 7F) The expression level and the percentage of pixels in all clusters (marker genes for each cluster) for RNA data in spatial-ATAC-RNA-seq. (FIG. 7G) Dot plot showing the identification of positive TF regulators. (FIG. 7H) Spatial mapping of deviation scores for selected TF motifs. (FIG. 7I) Annotation of marker peaks in different clusters. (FIG. 7J) GREAT enrichment analysis of marker peaks in different clusters (Binomial and hypergeometric tests).

FIG. 8A through FIG. 8F depicts further pseudotime analysis of radial glia and postmitotic premature neurons in spatial-ATAC-RNA-seq. (FIG. 8A) GO enrichment analysis for genes from FIG. 1G. (FIG. 8B-FIG. 8C) Pseudotime analysis from radial glia to postmitotic premature neurons with GAS (FIG. 8B) and gene expression (FIG. 8C). (FIG. 8D) Monocle2 analyses showing different states in (FIG. 8B). (FIG. 8E) Heatmap of different states along the pseudotime trajectory. (FIG. 8F) GO analysis of genes in red box of FIG. 8E (One-sided version of Fisher's exact test, p-value was adjusted for multiple comparisons by Benjamini & Hochberg method).

FIG. 9A through FIG. 9D depicts further analysis of spatial-ATAC-RNA-seq for P22 mouse brain. (FIG. 9A) Spatial mapping of gene activity scores and gene expression for selected marker genes in spatial-ATAC-RNA-seq. (FIG. 9B) Genome track visualization of marker genes with peak-to-gene links for distal regulatory elements and peak co-accessibility. (FIG. 9C) Spatial mapping of cell types identified by label transfer from scATAC-seq (Li, 2021, Nature, 598:129-136) to ATAC data. IT: intratelencephalic. PT: pyramidal tract. NP: near-projecting. CT: corticothalamic. L: layer. (FIG. 9D) Spatial mapping of cell types identified by label transfer from scRNA-seq (Zeisel, 2018, Cell, 174:999-1014) to RNA data.

FIG. 10A through FIG. 10D depict further analysis of P22 mouse brain in spatial-ATAC-RNA-seq. (FIG. 10A) Candidate TF regulators of Sox2, Pax6, and Mobp. Highlighted points are TFs with abs (regulation score)≥1 (−log 10 scale) (Kartha, 2022, Cell, 2:100166), with all other TFs shown in gray (Z test). (FIG. 10B) Spatial mapping of deviation scores for selected TF motifs. (FIG. 10C) Heatmaps of peak-to-gene links in spatial-ATAC-RNA-seq. (FIG. 10D) The number of significantly correlated peaks for each gene.

FIG. 11A through FIG. 11G depictsspatial chromatin accessibility and transcriptome co-sequencing of P21mouse brain. (FIG. 11A-FIG. 11C) Spatial distribution and UMAP of all the clusters for ATAC (FIG. 11A), RNA (FIG. 11B), and joint clustering of ATAC and RNA (FIG. 11C) in spatial-ATAC-RNA-seq for the mouse brain. Pixel size, 20 μm. Scale bar, 1 mm. (FIG. 11D) Nissl-stained image from an adjacent tissue section of P21 mouse brain. Scale bar, 1 mm. (FIG. 11E) Integration of ATAC data in spatial-ATAC-RNA-seq with scATAC-seq data (Li, 2021, Nature, 598:129-136) from mouse brain. (FIG. 11F) Integration of RNA data in spatial-ATAC-RNA-seq with scRNA-seq data (Zeisel, 2018, Cell, 174:999-1014) from mouse brain. (FIG. 11G) Spatial mapping of GAS and gene expression for selected marker genes in different clusters for ATAC and RNA in spatial-ATAC-RNA-seq.

FIG. 12A through FIG. 12H depict further analysis for spatial-CUT&Tag-RNA-seq with P22 mouse brain. (FIG. 12A) Integration of CUT&Tag (H3K4me3) data in spatial-CUT&Tag-RNA-seq with scCUT&Tag (H3K4me3) data (Bartosovic, 2021, Nature Biotechnology, 39:825-835) from mouse brain. (FIG. 12B) Integration of RNA data in spatial-CUT&Tag (H3K27me3)-RNA-seq, spatial-CUT&Tag (H3K27ac)-RNA-seq, and spatial-CUT&Tag (H3K4me3)-RNA-seq with scRNA-seq data (Zeisel, 2018, Cell, 174:999-1014) from mouse brain. (FIG. 12C-FIG. 12E) Integration of RNA data in spatial-CUT&Tag (H3K27me3)-RNA-seq (FIG. 12C), RNA data in data in spatial-CUT&Tag (H3K27ac)-RNA-seq (FIG. 12D), and RNA data in spatial-CUT&Tag (H3K4me3)-RNA-seq (FIG. 12E) with scRNA-seq data (Zeisel, 2018, Cell, 174:999-1014) from mouse brain. (FIG. 12F) Spatial mapping of cell types identified by label transfer from scRNA-seq (Zeisel, 2018, Cell, 174:999-1014) to RNA data in spatial-CUT&Tag (H3K27me3)-RNA-seq. (FIG. 12G) Spatial mapping of cell types identified by label transfer from scRNA-seq (Zeisel, 2018, Cell, 174:999-1014) to RNA (top) and from scRNA-seq (Zeisel, 2018, Cell, 174:999-1014) to CUT&Tag (H3K27ac, bottom) data in spatial-CUT&Tag (H3K27ac)-RNA-seq. (FIG. 12H) Spatial mapping of cell types identified by label transfer from scRNA-seq (Zeisel, 2018, Cell, 174:999-1014) to RNA (top) and from scRNA-seq (Zeisel, 2018, Cell, 174:999-1014) to CUT&Tag (H3K4me3, bottom) data in spatial-CUT&Tag (H3K4me3)-RNA-seq.

FIG. 13A through FIG. 13F depicts spatial epigenome and transcriptome co-sequencing and integrative analysis of P21 mouse brain. (FIG. 13A-FIG. 13C) Spatial distribution and UMAP of all the clusters for CUT&Tag (H3K27ac) (FIG. 13A), RNA (FIG. 13B), and joint clustering of CUT&Tag (H3K27ac) and RNA (FIG. 13C) in spatial-CUT&Tag-RNA-seq for the mouse brain. Pixel size, 20 μm. Scale bar, 1 mm. (FIG. 13D), Nissl-stained image from an adjacent tissue section of P21 mouse brain. Scale bar, 1 mm. (FIG. 13E), Integration of CUT&Tag (H3K27ac) data in spatial-CUT&Tag-RNA-seq with scCUT&Tag (H3K27ac) data (Bartosovic, 2021, Nature Biotechnology, 39:825-835) from mouse brain. (FIG. 13F) Integration of RNA data in spatial-CUT&Tag-RNA-seq with scRNA-seq data (Zeisel, 2018, Cell, 174:999-1014) from mouse brain.

FIG. 14A through FIG. 14H depicts spatial mapping of CSS, GAS, and gene expression of selected genes for P22 mouse brain. (FIG. 14A-FIG. 14G) Spatial mapping of CSS, GAS, and gene expression of Mal (FIG. 14A), Mag (FIG. 14B), Car2 (FIG. 14C), Grin2b (FIG. 14D), Syt1 (FIG. 14E), Gpr88 (FIG. 14F), and Ptprz1 (FIG. 14G) from ATAC and RNA in spatial-ATAC-RNA-seq, and CUT&Tag (H3K27me3, H3K27ac, or H3K4me3) and RNA in spatial-CUT&Tag-RNA-seq. (FIG. 14H) Spatial mapping of CSS and gene expression of Nav3, Sncb, Ablim2, and Gng7 in spatial-CUT&Tag (H3K27me3)-RNA-seq.

FIG. 15A through FIG. 15C depict further analysis of human hippocampus in spatial-ATAC-RNA-seq. (FIG. 15A) Spatial mapping of cell types identified by label transfer from scRNA-seq (Franjic, 2022, Neuron, 110:452-469) to RNA data in spatial-ATAC-RNA-seq for human hippocampus. (FIG. 15B) Dot plot showing the identification of positive TF regulators. (FIG. 15C) Spatial mapping of deviation scores for selected TF motifs in spatial-ATAC-RNA-seq.

FIG. 16A through FIG. 16D depict quality control metrics for spatial-ATAC-RNA-seq and spatial-CUT&Tag-RNA-seq datasets. (FIG. 16A) Scatterplots showing the TSS enrichment score vs unique nuclear fragments per pixel. (FIG. 16B) Nissl-stained image from an adjacent tissue section of P21 mouse brain. The grid has an interval of 20 μm. Scale bar, 200 μm. (FIG. 16C) The insert size distribution of ATAC or CUT&Tag fragments in spatial-ATAC-RNA-seq or spatial-CUT&Tag-RNA-seq. (FIG. 16D) The enrichment of ATAC or CUT&Tag reads around TSSs in spatial-ATAC-RNA-seq or spatial-CUT&Tag-RNA-seq.

FIG. 17A through FIG. 17E depict quality control metrics for spatial-ATAC-RNA-seq and spatial-CUT&Tag-RNA-seq datasets. (FIG. 17A) Comparison of TSS fragments and fraction of mitochondrial fragments in spatial-ATAC-RNA-seq and spatial-CUT&Tag-RNA-seq. (FIG. 17B) Comparison of number of unique fragments, TSS fragments, fraction of reads in peaks (FRiP), and fraction of mitochondrial fragments between biological replicates for spatial-ATAC-RNA-seq and spatial-CUT&Tag (H3K27ac)-RNA-seq. (FIG. 17C) Gene and UMI count distribution between biological replicates for spatial-ATAC-RNA-seq and spatial-CUT&Tag (H3K27ac)-RNA-seq. The box plots show the median (centre line), the first and third quartiles (box limits), and 1.5× the interquartile range (whiskers). (FIG. 17D) The reproducibility of spatial-ATAC-RNA-seq between biological replicates on ATAC data (left) and RNA data (right) for P21 mouse brain. (FIG. 17E) The reproducibility of spatial-CUT&Tag-RNA-seq between biological replicates on CUT&Tag data (left) and RNA data (right) for P21 mouse brain.

FIG. 18A through FIG. 18C depict benchmarking of data quality in spatial-ATAC-RNA-seq. (FIG. 18A) Comparison of transcriptional profiles between RNA in spatial-ATAC-RNA-seq and the ENCODE RNA-Seq data in brain of mouse embryo. (FIG. 18B) Aggregated spatial chromatin accessibility profiles in spatial-ATAC-RNA-seq recapitulated ENCODE ATAC-seq profiles in brain of mouse embryo. (FIG. 18C) Venn diagrams showing the overlap of peaks from ATAC in spatial-ATAC-RNA-seq and ENCODE ATAC-seq in brain of mouse embryo.

FIG. 19A through FIG. 19B depict further analysis of spatial-ATAC-RNA-seq for E13 mouse embryo. (FIG. 19A) GO enrichment analysis in selected RNA clusters from spatial-ATAC-RNA-seq for E13 mouse embryo (One-sided version of Fisher's exact test, p-value was adjusted for multiple comparisons by Benjamini & Hochberg method). (FIG. 19B) The number of significantly correlated peaks for each gene.

FIG. 20A through FIG. 20C depict further analysis of spatial-ATAC-RNA-seq for P22 mouse brain. (FIG. 20A) Ribbon plot showing the relationship between ATAC and RNA clusters in spatial-ATAC-RNA-seq. (FIG. 20B) Annotation of marker peaks in different clusters. (FIG. 20C) GREAT enrichment analysis of marker peaks in different clusters (Hypergeometric test).

FIG. 21A through FIG. 21C depict spatial mapping of CSS, GAS, and gene expression of selected genes in spatial-CUT&Tag-RNA-seq for P22 mouse brain. (FIG. 21A-FIG. 21C) Spatial mapping of CSS or GAS, and gene expression for selected marker genes in spatial-CUT&Tag (H3K27me3)-RNA-seq (FIG. 21A), spatial-CUT&Tag (H3K27ac)-RNA-seq (FIG. 21B), and spatial-CUT&Tag (H3K4me3)-RNA-seq (FIG. 21C).

FIG. 22A through FIG. 22C depict further analysis for spatial-CUT&Tag-RNA-seq with P22 mouse brain. (FIG. 22A) Genome track visualization of marker genes with peak-to-gene links for distal regulatory elements and peak co-accessibility. (FIG. 22B) Heatmaps of peak-to-gene links in spatial-CUT&Tag (H3K27ac)-RNA-seq. (FIG. 22C) The layers identified by Belayer from the top right mapping region of the P22 mouse brain. Evaluate the consistency between the two Belayer results (right) by adjusted rand index (ARI): ARI=0.915.

FIG. 23 depicts GO enrichment analysis of P22 mouse brain. GO enrichment analysis of genes from FIG. 4A (One-sided version of Fisher's exact test, p-value was adjusted for multiple comparisons by Benjamini & Hochberg method).

FIG. 24A through FIG. 24B depict further analysis for epigenetic regulation of gene expression with P22 mouse brain. (FIG. 24A) Correlation analysis of H3K27me3 CSS and RNA gene expression in striatum. (FIG. 24B) GO enrichment analysis of genes from FIG. 24A (One-sided version of Fisher's exact test, p-value was adjusted for multiple comparisons by Benjamini & Hochberg method).

FIG. 25A and FIG. 25B depict further analysis for epigenetic regulation of gene expression with P22 mouse brain. (FIG. 25A) Correlation analysis of H3K27me3 CSS and RNA gene expression in superficial cortical layer. (FIG. 25B) GO enrichment analysis of genes from FIG. 25A (One-sided version of Fisher's exact test, p-value was adjusted for multiple comparisons by Benjamini & Hochberg method).

FIG. 26A and FIG. 26B depict further analysis for epigenetic regulation of gene expression with P22 mouse brain. (FIG. 26A) Correlation analysis of H3K27me3 CSS and RNA gene expression in deeper cortical layer. (FIG. 26B) GO enrichment analysis of genes from FIG. 26A (One-sided version of Fisher's exact test, p-value was adjusted for multiple comparisons by Benjamini & Hochberg method).

FIG. 27 depicts further analysis for epigenetic regulation of gene expression with P22 mouse brain. GO enrichment analysis of genes from FIG. 4B and FIG. 4C (One-sided version of Fisher's exact test, p-value was adjusted for multiple comparisons by Benjamini & Hochberg method).

FIG. 28A and FIG. 28B depict further analysis for epigenetic regulation of gene expression with P22 mouse brain. (FIG. 28A) Correlation analysis of H3K27me3 CSS and H3K27ac GAS in corpus callosum. (FIG. 28B) GO enrichment analysis of genes from (FIG. 28A) (One-sided version of Fisher's exact test, p-value was adjusted for multiple comparisons by Benjamini & Hochberg method).

FIG. 29A and FIG. 29B depict further analysis for epigenetic regulation of gene expression with P22 mouse brain. (FIG. 29A) Correlation analysis of H3K27me3 CSS and H3K4me3 GAS in corpus callosum. (FIG. 29B) GO enrichment analysis of genes from (a) (One-sided version of Fisher's exact test, p-value was adjusted for multiple comparisons by Benjamini & Hochberg method).

### DETAILED DESCRIPTION

The present invention relates generally to systems and methods for spatially resolved epigenomic and transcriptomic co-profiling at single-cell level directly in the original tissue specimen. The presently described systems and methods represents an advancement in the field of biomedical research with far-reaching impact in developmental biology, cancer research, immunology, cardiovascular disease study, histopathology, and therapeutic discovery.

The present disclosure takes advantage of new technologies for spatial epigenomics—high resolution and deterministic spatial ATAC-seq (hsrATAC-seq) and spatial-CUT&Tag-seq which can be combined with high resolution and deterministic spatial RNA-seq for co-mapping of chromatin accessibility or epigenomic profiling and transcriptomic profiling.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In some embodiments, the invention provides methods of transcriptomic and epigenomic co-profiling of samples by combining spatial epigenomic mapping with spatial RNA analysis. Exemplary spatial-omics methods that can be incorporated in the methods of the invention include, but are not limited to, those described in International Patent Application Publication No. WO2022147239A1, U.S. patent application Ser. No. 17/036,401 and in Liu et al, 2020, Cell, 183 (6): 1665-1681 each of which is incorporated by reference herein in its entirety.

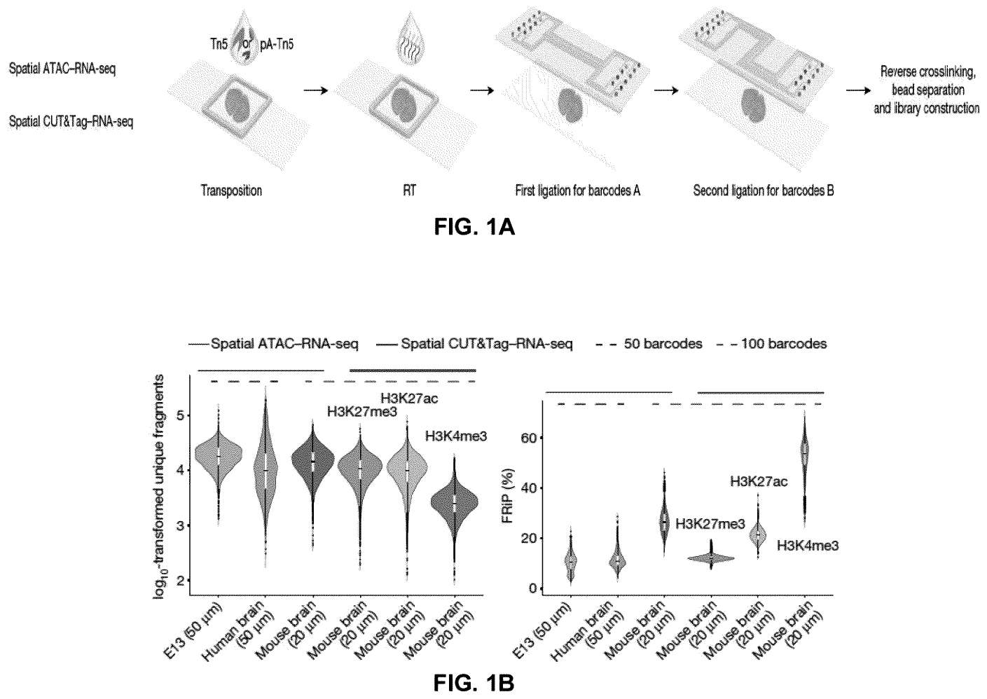

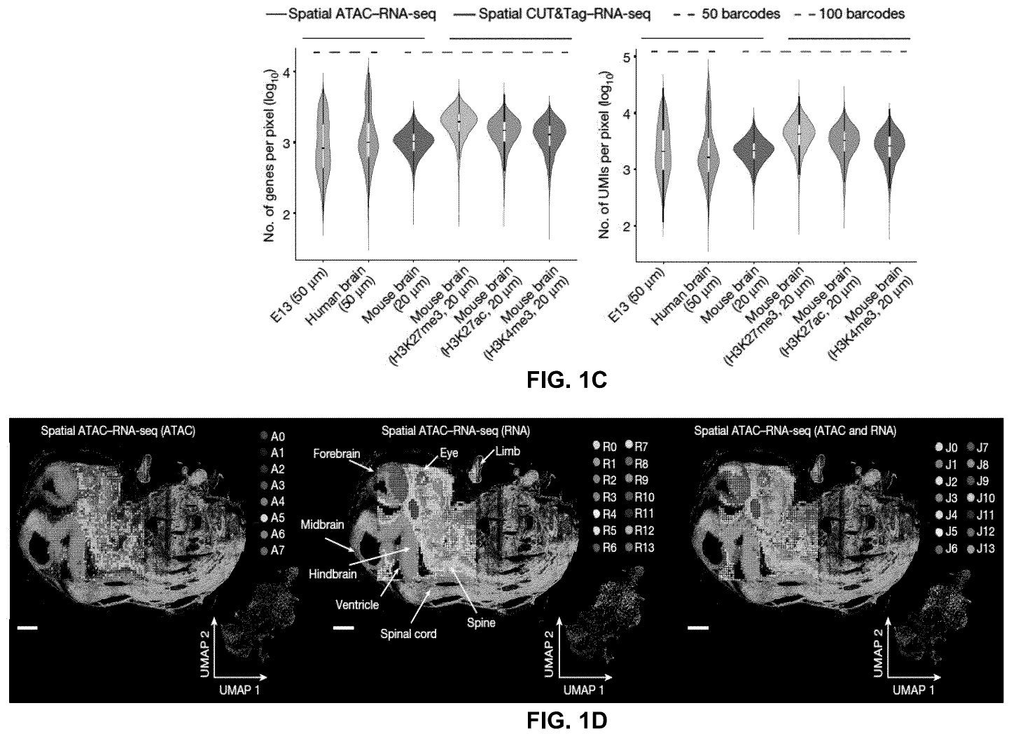

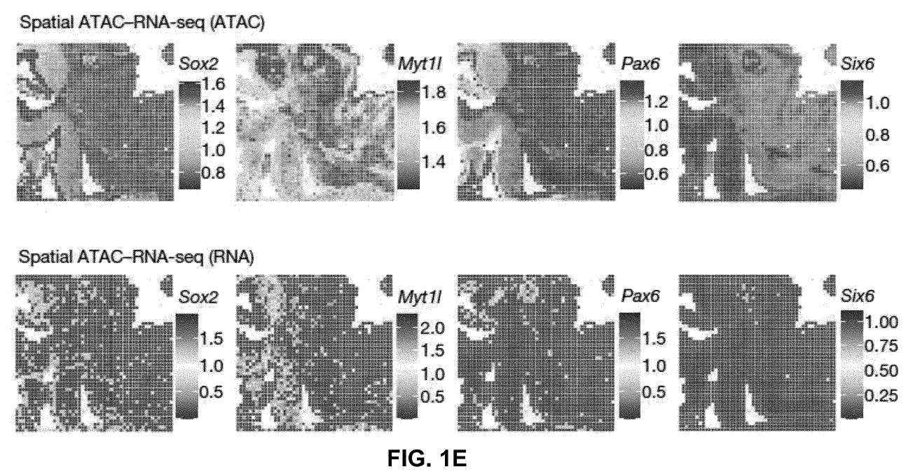

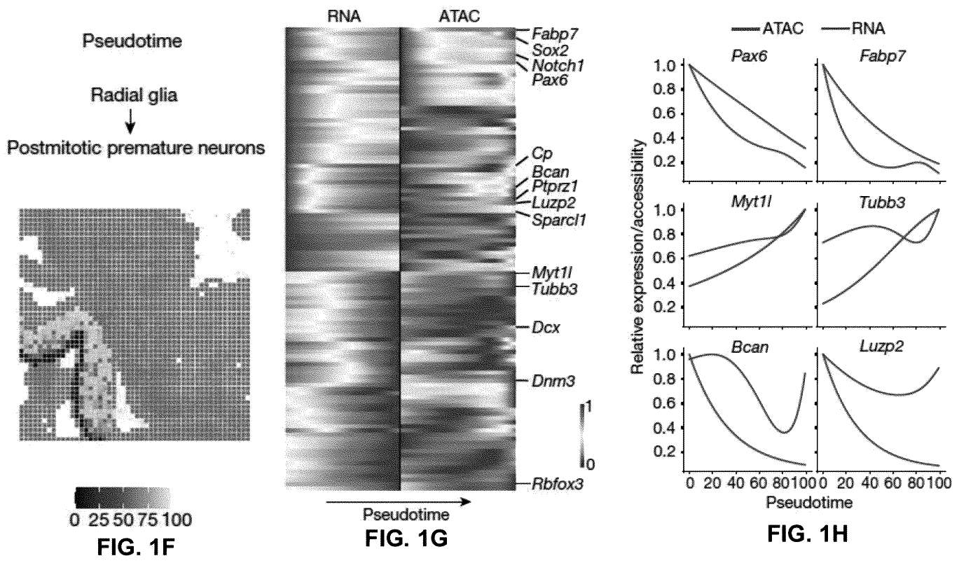

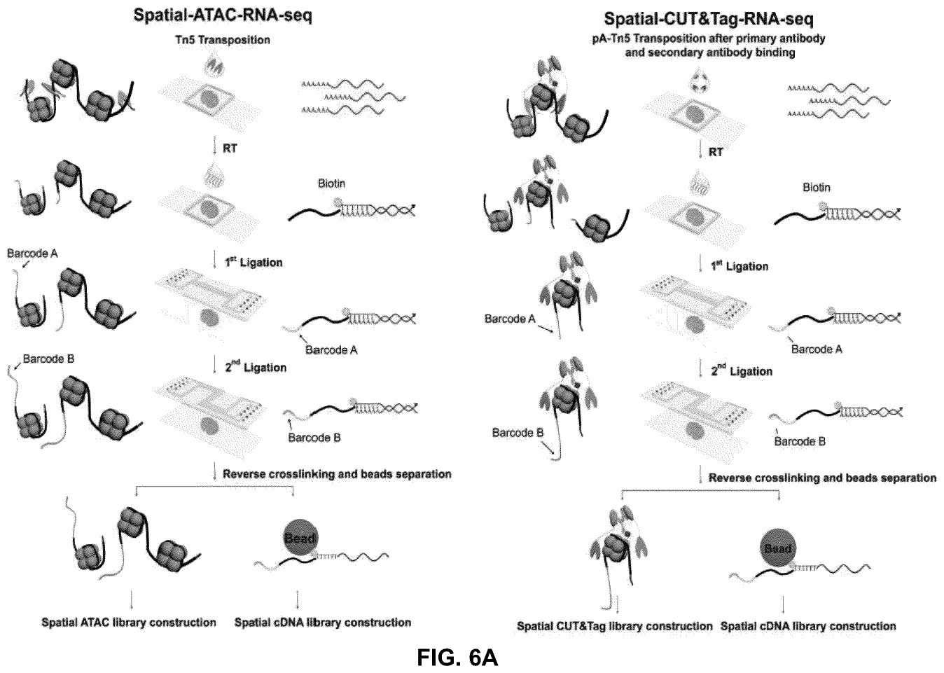

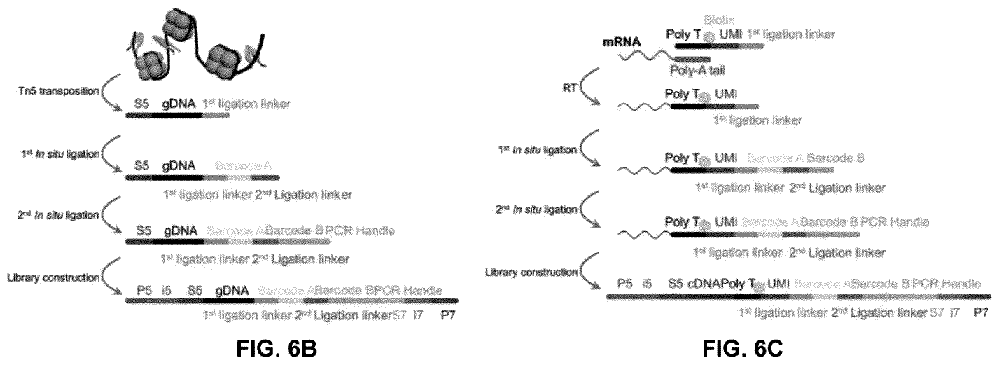

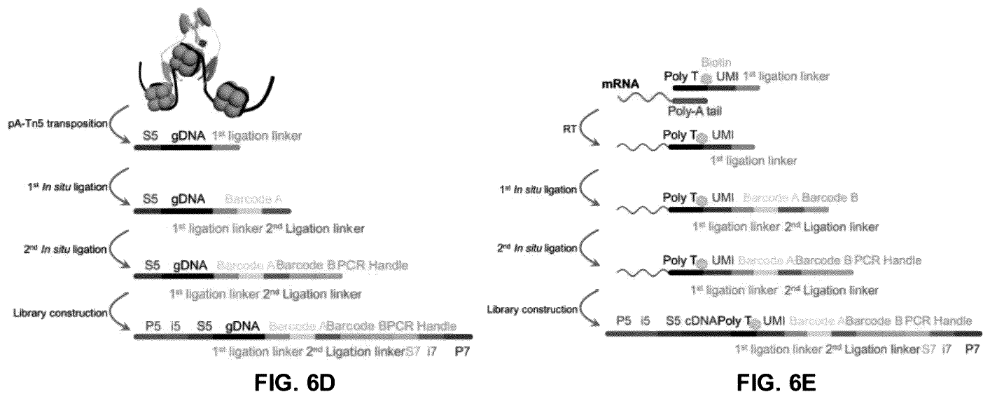

Exemplary schematic diagrams of the co-profiling methods of the invention are shown in FIG. 1A and FIG. 6. Spatial-ATAC-RNA-seq is shown schematically in FIG. 1A and FIG. 6A-FIG. 6C. In some embodiments, the method comprises the steps of applying a Tn5 transposition complex pre-loaded with a DNA adapter containing a universal ligation linker that can be inserted into the transposase accessible genomic DNA loci to a fixed frozen tissue section, incubating the tissue section with a biotinylated DNA adapter containing a universal ligation linker and a poly-T sequence that binds to the poly-A trail of mRNAs to initiate reverse transcription (RT) in tissue, placing a microfluidic channel array chip on the tissue section to introduce a first set of spatial barcodes (referred to herein as barcode set A) that are then covalently linked to the universal ligation linker via templated ligation, applying a second microchip with microchannels perpendicular to the first flow direction to the sample to introduce spatial barcodes that are then ligated to a second set of spatial barcodes (referred to herein as barcode set B), resulting in a 2D grid of spatially barcoded tissue pixels defined by the unique combination of barcodes A and B, and releasing the spatially barcoded cDNA and genomic DNA (gDNA) fragments by reversing the crosslinking. In some embodiments, cDNAs are enriched with streptavidin beads and gDNA fragments are retained in the supernatant. In some embodiments, libraries of gDNA and cDNA are constructed separately for next-generation sequencing (NGS).

Spatial-ATAC-RNA-seq is shown schematically in FIG. 1A and FIG. 6A-FIG. 6E. In some embodiments, the method of Spatial-CUT&Tag-RNA-seq comprises a step of applying an antibody against specific histone modification (i.e., H3K27me3, H3K27ac, or H3K4me3) to the tissue section and then applying a protein A tethered Tn5-DNA complex to perform co-assay of cleavage under targets and tagmentation (CUT&Tag), incubating the tissue section with a biotinylated DNA adapter containing a universal ligation linker and a poly-T sequence that binds to the poly-A trail of mRNAs to initiate reverse transcription (RT) in tissue, placing a microfluidic channel array chip on the tissue section to introduce a first set of spatial barcodes (referred to herein as barcode set A) that are then covalently linked to the universal ligation linker via templated ligation, applying a second microchip with microchannels perpendicular to the first flow direction to the sample to introduce spatial barcodes that are then ligated to a second set of spatial barcodes (referred to herein as barcode set B), resulting in a 2D grid of spatially barcoded tissue pixels defined by the unique combination of barcodes A and B, and releasing the spatially barcoded cDNA and genomic DNA (gDNA) fragments by reversing the crosslinking. In some embodiments, cDNAs are enriched with streptavidin beads and gDNA fragments are retained in the supernatant. In some embodiments, libraries of gDNA and cDNA are constructed separately for next-generation sequencing (NGS).

In one embodiment, the method comprises the steps of: placing a first microfluidic chip with parallel channels directly against tissue sample slide to be analyzed, contacting the sample with a transposase assembled with a DNA oligo sequence that serves as a ligation linker, flowing a first set of unique DNA barcodes (A₁-Aᵢ, wherein ᵢ is an integer between 1 and 1001) across the channels of the microfluidic chip in a first direction (A), ligating the first barcode set to the ligation linker, washing, removing the first microfluidic chip, applying a second microfluidic chip, wherein the second microfluidic chip is placed such that the flow direction is perpendicular to the flow direction of the first chip (A), flowing a second set of unique DNA barcodes (B₁-Bⱼ, wherein ⱼ is an integer between 1 and 1001) across the channels of the microfluidic chip in a second direction (B) which is perpendicular to the first direction (A), and ligating the second set of barcodes to the first barcode set. In some embodiments, the method further comprises lysing the cells, retrieving the spatially barcoded DNA molecules and preparing a NGS sequencing library from the spatially barcoded DNA molecules. In one embodiment, the method further includes a step of permeabilization prior to contacting the sample with the primary antibody. For example, in one embodiment, the sample is permeabilized with NP40-Digitonin buffer prior to contacting the sample with the transposase. In one embodiment, the transposase is a fusion protein of hyperactive Tn5 transposase and protein A.

In one embodiment, the method comprises the steps of: placing a first microfluidic chip with parallel channels (e.g., 20 or 50 μm in width) directly against tissue sample slide to be analyzed, contacting the sample with one or more antibodies specific for an epigenomic marker, contacting the sample with a secondary antibody and a transposase assembled with a DNA oligo sequence that serves as a ligation linker, flowing a first set of unique DNA barcodes (A₁-Aᵢ, wherein ᵢ is an integer between 1 and 1001) across the channels of the microfluidic chip in a first direction (A), ligating the first barcode set to the ligation linker, washing, removing the first microfluidic chip, applying a second microfluidic chip, wherein the second microfluidic chip is placed such that the flow direction is perpendicular to the flow direction of the first chip (A), flowing a second set of unique DNA barcodes (B₁-Bⱼ, wherein ⱼ is an integer between 1 and 1001) across the channels of the microfluidic chip in a second direction (B) which is perpendicular to the first direction (A), and ligating the second set of barcodes to the first barcode set. In some embodiments, the method further comprises lysing the cells, retrieving the spatially barcoded DNA molecules and preparing a NGS sequencing library from the spatially barcoded DNA molecules. In one embodiment, the method further includes a step of permeabilization prior to contacting the sample with the primary antibody. For example, in one embodiment, the sample is permeabilized with NP40-Digitonin buffer prior to contacting the sample with the primary antibody. In one embodiment, the transposase is a fusion protein of hyperactive Tn5 transposase and protein A.

In one embodiment, the method of the invention incorporates a DNA ligation adaptor or DNA barcode sequence, or a combination thereof, onto a nucleic acid molecule comprising an epigenomic mark of interest using a “cut and tag” method or “tagmentation.” As used herein, the term “tagmentation” refers to the modification of DNA by a transposome complex comprising transposase enzyme complexed with adaptors comprising transposon end sequence. Tagmentation results in the simultaneous fragmentation of the target DNA molecule comprising the epigenomic mark of interest and ligation of the adaptors to the 5′ ends of both strands of duplex fragments. Following a purification step to remove the transposase enzyme, additional sequences (e.g., barcodes) can be added to the ends of the adapted fragments, for example by PCR, ligation, or any other suitable methodology known to those of skill in the art.

The method of the invention can use any transposase that can accept a transposase end sequence and fragment a target nucleic acid, attaching a transferred end, but not a non-transferred end. A “transposome” is comprised of at least a transposase enzyme and a transposase recognition site. In some such systems, termed “transposomes”, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”. In some such insertion events, one strand of the transposase recognition site may be transferred into the target nucleic acid.

Some embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35:785, 1983; Savilahti, H, et al., EMBO J., 14:4893, 1995). An exemplary transposase recognition site that forms a complex with a hyperactive Tn5 transposase (e.g., EZ-Tn5™ Transposase, Epicentre Biotechnologies, Madison, Wis.).

More examples of transposition systems that can be used with certain embodiments provided herein include Staphylococcus aureus Tn552 (Colegio et al., J. Bacteriol., 183:2384-8, 2001; Kirby C et al., Mol. Microbiol., 43:173-86, 2002), Ty1 (Devine & Boeke, Nucleic Acids Res., 22:3765-72, 1994 and International Publication WO 95/23875), Transposon Tn7 (Craig, N L, Science. 271: 1512, 1996; Craig, N L, Review in: Curr Top Microbiol Immunol., 204:27-48, 1996), Tn/O and IS10 (Kleckner N, et al., Curr Top Microbiol Immunol., 204:49-82, 1996), Mariner transposase (Lampe D J, et al., EMBO J., 15:5470-9, 1996), Tcl (Plasterk R H, Curr. Topics Microbiol. Immunol., 204:125-43, 1996), P Element (Gloor, G B, Methods Mol. Biol., 260:97-114, 2004), Tn3 (Ichikawa & Ohtsubo, J Biol. Chem. 265:18829-32, 1990), bacterial insertion sequences (Ohtsubo & Sekine, Curr. Top. Microbiol. Immunol. 204:1-26, 1996), retroviruses (Brown, et al., Proc Natl Acad Sci USA, 86:2525-9, 1989), and retrotransposon of yeast (Boeke & Corces, Annu Rev Microbiol. 43:403-34, 1989). More examples include ISS, Tn10, Tn903, IS911, and engineered versions of transposase family enzymes (Zhang et al., 2009, PLOS Genet. 5: e1000689. Epub 2009 Oct. 16; Wilson C. et al (2007) J. Microbiol. Methods 71:332-5).

In one embodiment, the transposase is hyperactive Tn5 transposase tethered to protein A.

In one embodiment, the transposase is linked to a methylation sensitive restriction enzyme. Methylation sensitive restriction enzymes (MSREs) include, but are not limited to, Aat II, Acc II, Aor13H I, Aor51H I, BspT104 I, BssH II, Cfr10 I, Cla I, Cpo I, Eco52 I, Hae II, Hap II, Hha I, Mlu I, Nae I, Not I, Nru I, Nsb I, PmaC I, Psp1406 I, Pvu I, Sac II, Sal I, Sma I, and SnaB I.

In one embodiment, the tagmentation reaction is allowed to proceed for at least 10 minutes, at least 15 minutes, at least 20 minutes, at least 25 minutes, at least 30 minutes or for more than 30 minutes prior to flowing the first barcode set through the fluidic microchip.

In one embodiment, the concentration of transposome used for the tagementation reaction is between 1 μl and 20 μl. For example, in one embodiment, an 8 μl Tn5 transposome is assembled comprising 2 μl DNA oligo, 4 μl EZ-Tn5 Transposase (1 U/μl), and 2 μl glycerol). Before the tagmentation reaction, the Tn5 transposome is mixed with Tagment DNA buffer, 1×PBS, 10% Tween-20, 1% Digitonin to a total of 200 μl. In one embodiment, tagmentation is performed using a reaction time of at least 15, at least 20, at least 25, at least 30 or more than 30 minutes. In one embodiment, tagmentation is performed using 8 μl Tn5 transposome with a reaction time of 30 minutes.

### Epigenomic Marker Identification

In some embodiments, the methods of the invention include barcoding a nucleic acid molecule containing an epigenomic marker of interest in a biological sample. In some embodiments, the method includes the use of a primary antibody specific for binding to the epigenomic marker of interest. Non-limiting examples of antibodies include whole antibodies, Fab antibody fragments, F(ab′)₂ antibody fragments, monospecific Fab₂ fragments, bispecific Fab₂ fragments, trispecific Fab₃ fragments, single chain variable fragments (scFvs), bispecific diabodies, trispecific diabodies, scFv-Fc molecules, nanobodies, and minibodies.

In one embodiment, the primary antibody for use in the methods of the invention is specific for an epigenomic marker. Exemplary epigenomic markers that can be identified using the method of the invention include, but are not limited to, H2AK5ac, H2AK9ac, H2BK120ac, H2BK12ac, H2BK15ac, H2BK20ac, H2BK5ac, H2Bub, H3, H3ac, H3K14ac, H3K18ac, H3K23ac, H3K23me2, H3K27me1, H3K27me2, H3K36ac, H3K36me1, H3K36me2, H3K4ac, H3K56ac, H3K79me1, H3K79me3, H3K9acS10ph, H3K9me2, H3S10ph, H3T11ph, H4, H4ac, H4K12ac, H4K16ac, H4K5ac, H4K8ac, H4K91ac, H3F3A, H3K27me3, H3K36me3, H3K4me1, H3K79me2, H3K9me1, H3K9me2, H3K9me3, H4K20me1, H2AFZ, H3K27ac, H3K4me2, H3K4me3, and H3K9ac. Exemplary primary antibodies specific for epigenomic markers include, but are not limited to: (accession numbers from encodeproject.org) ENCAB841KJH, ENCAB000AOZ, ENCAB000APA, ENCAB000AOY, ENCAB000ARP, ENCAB000AQJ, ENCAB000ASI, ENCAB000AOS, ENCAB000AOR, ENCAB000APJ, ENCAB000API, ENCAB000ARU, ENCAB050QKP, ENCAB000AQK, ENCAB000AOT, ENCAB928LTI, ENCAB788ZME, ENCAB928HBB, ENCAB417DUO, ENCAB000AHF, ENCAB296TBH, ENCAB000APH, ENCAB000APG, ENCAB000ARW, ENCAB188IXL, ENCAB039IRN, ENCAB000AOK, ENCAB000AOL, ENCAB960XYH, ENCAB000ARX, ENCAB000ARY, ENCAB000ASZ, ENCAB602YNP, ENCAB205THQ, ENCAB375PDS, ENCAB931TIC, ENCAB961FBP, ENCAB750SJL, ENCAB453MST, ENCAB592AAE, ENCAB638MGM, ENCAB382YEO, ENCAB127FOW, ENCAB790SCK, ENCAB000ASH, ENCAB000ASJ, ENCAB121PMJ, ENCAB470FGK, ENCAB056ZFO, ENCAB000AOM, ENCAB000AOO, ENCAB000AON, ENCAB231VKB, ENCAB458UGW, ENCAB502YEA, ENCAB000ANJ, ENCAB829JCF, ENCAB002YEX, ENCAB093UKQ, ENCAB376DXS, ENCAB783AQT, ENCAB062SHF, ENCAB172ZWF, ENCAB638TXJ, ENCAB113TJV, ENCAB630GBO, ENCAB000AQQ, ENCAB529WLG, ENCAB150MLG, ENCAB255ALZ, ENCAB862RIQ, ENCAB327ADQ, ENCAB000AQT, ENCAB413BOQ, ENCAB498DNV, ENCAB093TAW, ENCAB151HMS, ENCAB000ARR, ENCAB000ARQ, ENCAB846BDR, ENCAB864KQT, ENCAB647DFQ, ENCAB000ART, ENCAB000ARS, ENCAB000APB, ENCAB494QXU, ENCAB723WFC, ENCAB984FPK, ENCAB738OTL, ENCAB844TLA, ENCAB771AMN, ENCAB643NJW, ENCAB219DGO, ENCAB155VEG, ENCAB036YAO, ENCAB268VLH, ENCAB009VWX, ENCAB000AQY, ENCAB266AZH, ENCAB000AUP, ENCAB000AQZ, ENCAB000ANB, ENCAB000ATC, ENCAB000ASA, ENCAB694MYM, ENCAB000AUT, ENCAB900FRR, ENCAB000ASD, ENCAB000ASC, ENCAB000ASB, ENCAB000AXZ, ENCAB000AXS, ENCAB323UEU, ENCAB000ADT, ENCAB169CDD, ENCAB782COR, ENCAB000ATF, ENCAB000ANC, ENCAB000ARI, ENCAB000ARJ, ENCAB000BLC, ENCAB000BLA, ENCAB000BLB, ENCAB910BYC, ENCAB773ECH, ENCAB570ZTO, ENCAB261ELA, ENCAB661HUV, ENCAB405MHV, ENCAB582RBY, ENCAB000ARD, ENCAB000AQW, ENCAB211WTE, ENCAB861ENQ, ENCAB000ADV, ENCAB360BDG, ENCAB523NUQ, ENCAB000AQB, ENCAB000BKT, ENCAB000APZ, ENCAB000AQC, ENCAB000AQD, ENCAB000ASN, ENCAB000ADU, ENCAB000AQE, ENCAB000ATB, ENCAB000AUW, ENCAB000AQF, ENCAB000AND, ENCAB000AQG, ENCAB000ARH, ENCAB000BKX, ENCAB000BSH, ENCAB543RHW, ENCAB027VOE, ENCAB539BDB, ENCAB969VGQ, ENCAB256MFX, ENCAB093ZAC, ENCAB663IEY, ENCAB650MWL, ENCAB472HKJ, ENCAB000ADW, ENCAB249ROX, ENCAB644AJI, ENCAB491AYZ, ENCAB000ARZ, ENCAB000APR, ENCAB000APS, ENCAB000ADX, ENCAB000ATH, ENCAB000AYB, ENCAB378MIH, ENCAB845ARK, ENCAB000AQU, ENCAB208AUK, ENCAB000ANE, ENCAB000ARE, ENCAB000APP, ENCAB000APO, ENCAB775EVT, ENCAB483QLF, ENCAB913CFY, ENCAB627HBE, ENCAB001LDA, ENCAB000AOQ, ENCAB000ANI, ENCAB000ANH, ENCAB000AQP, ENCAB004CMB, ENCAB352FQM, ENCAB180QII, ENCAB000APT, ENCAB000ANP, ENCAB681ELK, ENCAB449CFZ, ENCAB778TBN, ENCAB172IHG, ENCAB929ZIJ, ENCAB027OJQ, ENCAB769IVA, ENCAB164QXS, ENCAB890YOB, ENCAB691OYV, ENCAB499JWV, ENCAB292IFT, ENCAB130GEM, ENCAB369JSU, ENCAB003LHL, ENCAB000ANQ, ENCAB679IZV, ENCAB048FFK, ENCAB000AUR, ENCAB000APW, ENCAB000APV, ENCAB000APY, ENCAB000APU, ENCAB000AXW, ENCAB000APX, ENCAB000ANX, ENCAB000ANY, ENCAB000ATI, ENCAB000AQS, ENCAB000ARG, ENCAB000ARF, ENCAB972UJU, ENCAB027NDF, ENCAB343QJE, ENCAB000ANZ, ENCAB000AUS, ENCAB000AQV, ENCAB629MIV, ENCAB000AQI, ENCAB000BKS, ENCAB000ASY, ENCAB000AOU, ENCAB000BSK, ENCAB721ICQ, ENCAB343GLF, ENCAB749NPH, ENCAB943WPC, ENCAB661VDQ, ENCAB101KHB, ENCAB974EBC, ENCAB372RPK, ENCAB502OHI, ENCAB557LLB, ENCAB088TFM, ENCAB037IXK, ENCAB003HJF, ENCAB793BZS, ENCAB228OWC, ENCAB000ADS, ENCAB654QHT, ENCAB000AQM, ENCAB137OAB, ENCAB000AQN, ENCAB000APD, ENCAB000APF, ENCAB000APE, ENCAB000APC, ENCAB000ANA, ENCAB000BKR, ENCAB000BSI, ENCAB749UMK, ENCAB638ANC, ENCAB813FEB, ENCAB492DPX, ENCAB346FTT, ENCAB420YAH, ENCAB716RFU, ENCAB382AVR, ENCAB367DWC, ENCAB413RSR, ENCAB000AOP, ENCAB000ADY, ENCAB000ASO, ENCAB000AUX, ENCAB000ANF, ENCAB000BSJ, ENCAB725RFE, ENCAB610CEF, ENCAB008SYM, ENCAB170RJO, ENCAB582RSV, ENCAB385IEP, ENCAB081ENJ, ENCAB902NZL, ENCAB848NER, ENCAB682XRE, ENCAB388GOH, ENCAB884CKI, ENCAB000ARL, ENCAB008TOZ, ENCAB513PLB, ENCAB000ARB, ENCAB000ARO, ENCAB000ARC, ENCAB000ARA, ENCAB000ARK, ENCAB000ASG, ENCAB000AUU, ENCAB000ARM, ENCAB000ARN, ENCAB140BWE, ENCAB000ANU, ENCAB000AQR, ENCAB000AAA, ENCAB000ANL, ENCAB000APM, ENCAB000APL, ENCAB000ANG, ENCAB000ATA, ENCAB000AUV, ENCAB000APN, ENCAB000ANV, ENCAB000BKU, ENCAB000BKY, ENCAB000BLG, ENCAB000BLD, ENCAB000BLJ, ENCAB000BLH, ENCAB000BLE, ENCAB000BLI, ENCAB000BLF, ENCAB874PYE, ENCAB237XGS, ENCAB261POO, ENCAB576XIU, ENCAB851GAY, ENCAB000AOX, ENCAB000ANM, ENCAB000ANK, ENCAB000ANN, ENCAB000ANO, and ENCAB000ARV.

### Barcoded Polynucleotides

In some embodiments, the methods relate to contacting a sample with at least one set of barcoded polynucleotides. In some embodiments, the methods relate to contacting a sample with at least two sets of barcoded polynucleotides. In some embodiments, the number of unique barcoded polynucleotides in a set corresponds to the number of channels on a microfluidic chip. Therefore, in various embodiments, a set of barcoded polynucleotides comprises 5 to 1000 unique barcode sequences.

Non-limiting examples of barcoded polynucleotides (e.g., barcoded DNA) of the present disclosure a provided in Tables 4 and 5. In some embodiments, barcoded polynucleotides (e.g., of a first set of barcoded polynucleotides) include two ligation linker sequences, and a spatial barcode sequence, wherein the spatial barcode sequence is flanked on either side by a ligation linker sequence. In some embodiments, barcoded polynucleotides (e.g., of a second set of barcoded polynucleotides) include a ligation linker sequence, a spatial barcode sequence, and a sequence complementary to a PCR primer.

In one exemplary embodiment, for use with a microfluidic chip comprising 100 microchannels, a set of barcoded polynucleotides comprises 100 barcoded polynucleotides. Exemplary sets of 100 barcoded polynucleotides comprise set “A” barcodes of Table 4, comprising SEQ ID NO: 1-SEQ ID NO: 100. In one exemplary embodiment, for use with a microfluidic chip comprising 100 microchannels, a second set of barcoded polynucleotides comprises set “B” barcodes of Table 5, comprising SEQ ID NO: 101-SEQ ID NO:200.

A ligation linker sequence is any sequence complementary to a sequence of a ligation adaptor sequence or universal ligation linker, as provided herein. The length of a ligation linker sequence may vary. For example, a ligation linker sequence may have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, a ligation linker sequence may have a length of 5, 10, 15, 20, 25, 30, 35, 40, 45, or 50 nucleotides. Longer ligation linker sequences are contemplated herein. In some embodiments, a ligation linker sequence of a barcoded polynucleotide of one set (e.g., a first set) differ (e.g., have a different composition of nucleotides and/or a different length) from a ligation linker sequence of a barcoded polynucleotide of another set (e.g., a second set).

A barcode sequence is a unique sequence that can be used to distinguish a barcoded polynucleotide in a biological sample from other barcoded polynucleotides in the same biological sample. A spatial barcode sequence is a barcode sequence that is associated with a particular location in a biological sample (e.g., a tissue section mounted on a slide). The concept of “barcodes” and appending barcodes to nucleic acids and other proteinaceous and non-proteinaceous materials is known to one of ordinary skill in the art (see, e.g., Liszczak G et al. Angew Chem Int Ed Engl. 2019 Mar. 22; 58 (13): 4144-4162). Thus, it should be understood that the term “unique” is with respect to the molecules of a single biological sample and means “only one” of a particular molecule or subset of molecules of the sample. Thus, a “pixel” (also referred to as a “patch) comprising a unique spatially addressable barcoded conjugate (or a unique subset of spatially addressable barcoded conjugates) is the only pixel in the sample that includes that particular unique barcoded polynucleotide (or unique subset of barcoded polynucleotides), such that the pixel (and any molecule(s) within the pixel) can be identified based on that unique barcoded conjugate (or a unique subset of barcoded conjugates).

For example, in some embodiments, the polynucleotides of subset A1 (of Barcode A) are coded with a specific barcode sequence, while the polynucleotides of subsets A2, A3, A4, etc. are each coded with a different barcode sequence, each barcode specific to the subset. Likewise, the polynucleotides of subset B1 (of Barcode B) are coded with a specific barcode sequence, while the polynucleotides of subsets B2, B3, B4, etc. are each coded with a different barcode sequence, each barcode specific to the subset. Thus, each overlapping patch, which includes a unique combination of Barcode A subsets and Barcode B subsets, contains a unique composite barcode (Barcode A+Barcode B). For example, an overlapping pixel (patch) containing A1+B1 barcodes is uniquely coded relative to its neighboring overlapping patches, which contain A2+B1 barcodes, A1+B2 barcodes, A2+B2 barcodes, etc.

The length of a spatial barcode sequence may vary. For example, a spatial barcode sequence may have a length of 5 to 50 nucleotides (e.g., 5 to 40, 5 to 30, 5 to 20, 5 to 10, 10 to 50, 10 to 40, 10 to 30, or 10 to 20 nucleotides). In some embodiments, a spatial barcode sequence may have a length of 5, 10, 15, 20, 25, 30, 35, 40, 45, or 50 nucleotides. Longer spatial barcode sequences are contemplated herein.

In one embodiment, the method of the invention further comprises contacting the sample with one or more additional barcode sequence (e.g., a “zone” barcode sequence to distinguish specific regions or “zones” of a larger surface.) Therefore, in various embodiments, the methods include sequential ligation of at least one, two, three, four, five, or more than five unique barcode sequences to a target nucleic acid molecule. In one embodiment, each barcoded polynucleotide set comprises at least 10 barcoded polynucleotides.

### Universal Ligation Linkers

Also provided herein are universal ligation linkers, which may be a polynucleotide, for example, that includes (i) a first nucleotide sequence that is complementary to and/or binds to the linker sequence of the barcoded polynucleotides of a first set of barcoded polynucleotides, and (ii) a second nucleotide sequence that is complementary to and/or binds to the linker sequence of the barcoded polynucleotides of a second set of barcoded polynucleotides. The purpose of the universal ligation linkers is to serve as a bridge to join barcoded polynucleotides from two different sets (e.g., the first set comprising two ligation linker sequences flanking a spatial barcode sequence, and the second set comprising a ligation linker sequence, a spatial barcode sequence, and a sequence complementary to a PCR primer). The length of a universal ligation linker may vary. For example, a universal ligation linker may have a length of 10 to 100 nucleotides (e.g., 10 to 90, 10 to 80, 10 to 70, 10 to 60, 10 to 50, 10 to 40, 10 to 30, 10 to 20, 20 to 100, 20 to 90, 20 to 80, 20 to 70, 20 to 60, 20 to 50, 20 to 40, or 20 to 30 nucleotides). In some embodiments, a universal ligation linker may have a length of 10, 15, 20, 25, 30, 35, 40, 45, 50, 55, 60, 65, 70, 75, 80, 85, 90, 95, or 100 nucleotides. Longer universal ligation linkers are contemplated herein.

The universal ligation linkers are typically added to a biological sample following the delivery of a set of barcoded polynucleotides, although, in some embodiments, universal ligation linkers are annealed to the barcoded polynucleotides prior to delivery.

In some embodiments, the ligation adapter or universal ligation linker added to the 5′ and/or 3′ end of a nucleic acid during the method of the invention includes, but are not limited to, a nucleic molecule comprising a nucleotide sequence of SEQ ID NO:202 or SEQ ID NO:203, or a fragment thereof. In some embodiments, the ligation adapter or universal ligation linker added to the 5′ and/or 3′ end of a nucleic acid during the method of the invention includes, but are not limited to, a nucleic molecule for hybridization to a nucleotide sequence of SEQ ID NO:202 or SEQ ID NO:203, or a fragment thereof.

### Methods

In some embodiments, the methods comprise delivering to a biological tissue a first set of barcoded polynucleotides. A first set may include any number of barcoded polynucleotides. In some embodiments, a first set include 5 to 1000 barcoded polynucleotides. For example, a first set may comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. More than 1000 barcoded polynucleotides in a first set are contemplated herein.

In some embodiments, the method further includes a step of permeabilization prior to delivering the first set of barcoded polynucleotides, for example, through the first microfluidic device. Thus, in some embodiments, the methods comprise delivering to a biological tissue permeabilization reagents (e.g., detergents such as Triton-X 100 or Tween-20). In some embodiments, the methods comprise delivering to a biological tissue a first set of barcoded polynucleotides, and then delivering to the biological tissue permeabilization reagents.

In some embodiments, the methods comprise delivering to the biological sample a second set of barcoded polynucleotides. A second set may include any number of barcoded polynucleotides. In some embodiments, a second set include 5 to 1000 barcoded polynucleotides. For example, a first set may comprise 5 to 900, 5 to 800, 5 to 700, 5 to 600, 5 to 500, 5 to 400, 5 to 300, 5 to 200, 5 100, 10 to 1000, 10 to 900, 10 to 800, 10 to 700, 10 to 600, 10 to 500, 10 to 400, 10 to 300, 10 to 200, 20 to 1000, 20 to 900, 20 to 800, 20 to 700, 20 to 600, 20 to 500, 20 to 400, 20 to 300, 20 to 200, 50 to 1000, 50 to 900, 50 to 800, 50 to 700, 50 to 600, 50 to 500, 50 to 400, 50 to 300, or 50 to 200 barcoded polynucleotides. More than 1000 barcoded polynucleotides in a second set are contemplated herein.

In some embodiments, the methods comprise joining barcoded polynucleotides of the first set to barcoded polynucleotides of the second set. In some embodiments, the methods comprise exposing the biological sample to a ligation reaction, thereby producing a two-dimensional array of spatially addressable barcoded conjugates bound to molecules of interest, wherein the spatially addressable barcoded conjugates comprises a unique combination of barcoded polynucleotides from the first set and the second set.

In some embodiments, the methods comprise imaging the biological sample to produce a sample image. An optical microscope or a fluorescence microscope, for example, may be used to image the sample.

### Sequencing

In some embodiments, the methods include a sequencing step. For example, next generation sequencing (NGS) methods (or other sequencing methods) may be used to sequence the nucleic acid molecules recovered following cell lysis. In some embodiments, the methods comprise preparing an NGS library in vitro. Thus, in some embodiments, the methods comprise sequencing the library of barcoded nucleic acid molecules to produce sequencing reads. Other sequencing methods are known, and an example protocol is provided herein.

In some embodiments, the methods comprise constructing a spatial epigenomic map of the biological sample by matching the spatially addressable barcoded conjugates to corresponding sequencing reads. In some embodiments, the methods comprise identifying the location of the molecules of interest by correlating the spatial epigenomic map to the sample image.

### HSR Microfluidic-Based Systems

To achieve high spatial resolution in a biological context, a detector (e.g., microfluidic device) should profile single cells and resolve spatial features small enough to meaningfully image patterns in the spatial arrangement of single cells and groups of cells. An exemplary high spatial resolution microfluidic based system that can be utilized for the methods of the invention is described in detail in U.S. patent application Ser. No. 17/036,401 and in Liu et al, 2020, Cell, 183 (6): 1665-1681 each of which is incorporated by reference herein in its entirety.

Single-Cell Resolution. A detector can profile single cells if the detectors' pixels are of approximately equal or smaller size than the cells. Given mammalian cell sizes that range from approximately 5-20 microns (μm) in length, this entails utilizing a detector with pixels of approximately the same length. Although cell sizes vary within samples, and some cells may be larger and some smaller than detector pixels with a constant size, the inventors have found that by combining optical imaging with digital spatial reconstruction they can select those pixels that circumscribe a single cell in order to achieve true single-cell resolution, even if only for subset of a reconstructed image.

Imaging Multicellular Motifs. In addition to profiling individual cells, it is also useful to consider the ability of an imaging detector to resolve spatial features as being determined by the center-center distance between imaging pixels. This perspective becomes more relevant when examining structures or motifs comprising groups of cells rather than individual cells, such as developing organoids in mouse embryos, as shown in the Examples provided herein.

The standard criterion used in data processing in both the time and spatial domains is the Nyquist Criterion, which dictates that given a center-center distance of a certain number of microns, a detector can faithfully reproduce imaged spatial features only down to approximately twice that center-center distance. Given mammalian cell sizes that range from approximately 5-20 μm and that typically neighbor each other face-to-face, features of cell neighborhoods should vary over distances equal to one or more cell lengths. Thus, to resolve these features, a the HSR detector provided herein, in some embodiments, includes pixels with center-center distance between pixels of not more than several cell lengths, e.g., 10-50 μm.

Imaging systems with pixel sizes and center-center distances much larger than these values cannot profile single cells or resolve features characteristic of cells or multicellular features and therefore do not display HSR. For example, a detector with pixels with size of 1 millimeter would probe distance scales of size 1-2 mm or larger and would not resolve single cells or multicellular features. As the present disclosure described elsewhere herein, pixels much smaller than this range (e.g., less than one micron) result in unsuitable detectors because their mappable area becomes extremely small and logistical tasks (including reagent loading and delivery) become impractical to carry out. The inventors have found that there is a critical range for high-throughput HSR detection with channel width and pitch (near the region of interest) between approximately 2.5-50 μm, for example.

### Microfluidic Devices

Microfluidic devices (e.g., chips) may be used, in some embodiments, to deliver barcoded polynucleotides to a biological sample in a spatially defined manner. A system based on crossed microfluidic channels, such as those described here, have several key parameters that largely determine the spatial resolution and mappable area of the device. These include (1) the number of microfluidic channels (n/eta); (2) the microchannel width (w/omega), measured in microns, i.e., the width of the open space in each microfluidic channel (tissue beneath these open spaces is imaged); and (3) microchannel pitch (A/delta), measured in microns, i.e., the width of the closed space between the end of one channel and the start of another channel (tissue beneath these closed spaces is not imaged).

In some embodiments, the microfluidic devices provided herein include multiple microchannels characterized by a certain width, depth, and pitch. In some embodiments, the microfluidic devices of the invention achieve high spatial resolution at the single-cell level.

In one embodiment, the system of the invention comprises two microfluidic devices. For example, in one embodiment, a first device flows reagents left to right and is drawn as a series of rows, and a second device flows reagents from top to bottom and is drawn as a series of columns. The pixels of the detector comprise the overlap areas between the two sets of shapes, and as can be seen in the drawing such a geometry endows the squares with edge length w microns. As an illustrative example, assume a detection scheme that utilizes microfluidic devices with η=50, ω=10 microns, and Δ=10 microns. In some embodiments, the detector will feature pixels that are squares with edge length 10 microns, and the distance between squares in the horizontal and vertical directions is equal to 20 microns. This means it can profile single cells that are approximately 10 microns or larger and resolve spatial features (e.g., characteristics of cell neighborhoods) that are 40 microns or larger. In some embodiments, such microfluidic-based detectors will display certain performance characteristics determined by the design and the design parameters, including, but not limited to, the ability to profile individual cells; a minimum length scale of spatial feature reproduction; and the size of the mappable area.

Number of microchannels. In some embodiments, a first set of barcoded polynucleotides is delivered through a first microfluidic chip that comprises parallel microchannels positioned on a surface of the biological sample. In some embodiments, a first microfluidic chip comprises at least 5, at least 10, at least 20, at least 30, at least 40, or at least 50 parallel microchannels. In some embodiments, a first microfluidic chip comprises 5, 10, 20, 30, 40, or 50 parallel microchannels. In some embodiments, a first microfluidic chip comprises 5-1000 parallel microchannels (e.g., 5-10, 5-25, 5-50, 5-75, 10-25, 10-50, 10-75, 10-1000, 25-500, 25-200, 25-100, 50-200, or 50-100 parallel microchannels). In some embodiments, a second set of barcoded polynucleotides is delivered through a second microfluidic chip that comprises parallel microchannels that are positioned on the biological sample perpendicular to the direction of the microchannels of the first microfluidic chip. In some embodiments, a second microfluidic chip comprises at least 5, at least 10, at least 20, at least 30, at least 40, or at least 50 parallel microchannels. In some embodiments, a second microfluidic chip comprises 5-1000 parallel microchannels (e.g., 5-10, 5-25, 5-50, 5-75, 10-25, 10-50, 10-75, 10-1000, 25-500, 25-200, 25-100, 50-200, or 50-100 parallel microchannels).

Microchannel width. In some embodiments, a microchannel has a width of at least 5 μm (e.g., at least 5 μm, at least 10 μm, at least 15 μm, at least 20 μm, at least 25 μm, at least 30 μm, at least 35 μm, at least 40 μm, or at least 50 μm). In some embodiments, a microchannel has a width of 10 μm, 15 μm, 20 μm, 25 μm, 30 μm, 35 μm, 40 μm, 50 μm or more than 50 μm. In some embodiments, a microchannel has a width of 5 μm to 1000 μm (e.g., 10-500 μm, 10-100 μm, 20-200 μm, 20-100 μm).

In some embodiments, the microchannels have variable width. Variable channel width eases fluid flow through the microfluidic channels. For example, in one embodiment, a 50 μm device features 100 μm channels which shrink to 50 μm only near the region of interest. As another example, a 20 μm device's channels shrink to 100, 50, and then 20 μm near the region of interest. As yet another example, a 10 μm device's channels range from 100, 50, 25, and then 10 μm near the region of interest.

In some embodiments, a microchannel has a width of 20 μm to 1000 μm near the inlet and outlet ports and a width of 5 μm to 100 μm near the region of interest. For example, a microchannel may have a width of 100 μm near the inlet and outlet ports and width of 50 μm near the region of interest. As another example, a microchannel may have a width of 100 μm near the inlet and outlet ports and width of 20 μm near the region of interest. In some embodiments, a microchannel has a width of 50, 60, 70, 80, 90, 100, 110, 120, 130, 130, 140, or 150 μm near the inlet and outlet ports. In some embodiments, a microchannel has a width of 10, 20, 30, 40, or 50 μm near the region of interest.

In some embodiments, the microchannels are serpentine, allowing for the fluid to flow back and forth across a sample in a pattern. Use of serpentine microchannels can be used to apply a specific barcode sequence in a repeated pattern across a sample. In some embodiments a serpentine microfluidic device is combined with a non-serpentine microfluidic device which flows a second set of barcodes in a straight pattern and a third method of applying barcodes to specific non-overlapping zones, such that each tixel comprises a unique set of barcodes.

Microchannel height. In one embodiment, the microchannel height is approximately equal (e.g., within 10%) to the microchannel width. In some embodiments, a microchannel has a height of at least 10 μm (e.g., at least 15 μm, at least 20 μm, at least 25 μm, at least 30 μm, at least 35 μm, at least 40 μm, or at least 50 μm). In some embodiments, a microchannel has a height of 10 μm, 15 μm, 20 μm, 25 μm, 30 μm, 35 μm, 40 μm, or 50 μm). In some embodiments, a microchannel has a height of 10 μm to 150 μm (e.g., 10-125 μm, 10-100 μm, 25-150 μm, 25-125 μm, 25-100 μm, 50-150 μm, 50-125 μm, or 50-100 μm). These heights have been tested and shown to be sufficient to provide clearance above dust or tissue blockages, for example, and low enough to provide good sufficient rigidity and to prevent deformation of the channel during clamping and flow.

In some embodiments, a microchannel has a width of 10 μm and a height of 12-15 μm. In other embodiments, a microchannel has a width of 25 μm and a height of 17-22 μm. In yet other embodiments, a microchannel has a width of 50 μm and a height of 20-100 μm.

Microchannel pitch. The pitch is the distance between microchannels of a microfluidic device (e.g., chip). In some embodiments, the pitch of a microfluidic device is at least 10 μm (e.g., at least 15 μm, at least 20 μm, at least 25 μm, at least 30 μm, at least 35 μm, at least 40 μm, or at least 50 μm). In some embodiments, the pitch of a microfluidic device is at 10 μm, 15 μm, 20 μm, 25 μm, 30 μm, 35 μm, 40 μm, or 50 μm. In some embodiments, the pitch of a microfluidic device is at 10 μm to 150 μm (e.g., 10-125 μm, 10-100 μm, 25-150 μm, 25-125 μm, 25-100 μm, 50-150 μm, 50-125 μm, or 50-100 μm).

### Negative Pressure Systems

Many microfluidics platforms utilize positive pressure via syringe pumps, peristaltic pumps, and other types of positive pressure pumps whereby fluid is pumped from a reservoir into the device. Generally, a connection is made to interface the reservoir/pump assembly with the microfluidic device; often this takes the form of tubes terminating in pins that plug into inlet ports on the device. However, this type of system requires laborious and time-consuming fine-tuning of the assembly process associated with several drawbacks. For example, if the pins are inserted insufficiently deep into the inlet wells or the pin diameter is too small relative to the ports, then upon activation of the pumps, fluid pressure will eject the tube from the port. As another example, if the pins are inserted excessively deep into the wells, then upon activation of the pumps, fluid pressure will separate the microfluidic device from the glass substrate, resulting in leakage. While epoxying pins into ports and/or bonding the microfluidic device to the substrate via plasma bonding or thermal bonding might address the foregoing drawbacks, these strategies make it difficult to disassemble the system in a non-destructive way, resulting in component loss and are impractical when the substrate contains sensitive material, such as a tissue section, and/or antibodies.

The methods and devices provided herein, by contrast, overcome the drawbacks associated with existing microfluidic platforms by using, in some embodiments, a negative pressure system that utilizes a vacuum to pull liquid through the device from the back, rather than positive pressure to push it through the device from the front. This has several advantages, including, for example, (i) reducing the risk of leakage by pulling together the device and substrate and (ii) increasing efficiency and ease of use—the vacuum can be applied to all outlet ports, unlike pins, which must be inserted individually into each inlet port. Using a negative pressure system saves several hours per run of fine-tuning and pin assembly.

Thus, in some embodiments provided herein, the barcoded polynucleotides are delivered to a region of interest through a microfluidic device (e.g., chip) using negative pressure (vacuum). In some embodiments, delivery of a first set of barcoded polynucleotides is delivered through a first microfluidic device using a negative pressure system. In some embodiments, delivery of a second set of barcoded polynucleotides is delivered through a second microfluidic device using a negative pressure system.

### Inlet and Outlet Ports

In some embodiments the microfluidic devices having a common outlet port are vulnerable to backflow of reagents into the region of interest through incorrect microchannels, particularly during device disassembly. Such backflow can result in incorrect addressing of target molecules, resulting in an incorrect reconstruction of a spatial map of target molecules performed in later steps of the methods (e.g., after sequencing). To limit the possibility of reagent backflow, the microfluidic devices provided herein, in some embodiments, include microchannels that each have its own inlet port and outlet port. For example, in one embodiment, a microchannel device comprising 50 microchannels has 50 inlet ports and 50 outlet ports. In one embodiment, a microchannel device comprising 100 microchannels has 100 inlet ports and 100 outlet ports.

### Clamping

During initial experiments used to test the microfluidic devices and methods provided herein, frequent leakage of reagents occurred between channels on the region of interest. Convention clamping mechanisms proved cumbersome and introduced difficulties in addressing inlet and outlet ports. To address the issues identified, a new clamping mechanism was developed, which combines specific clamping parameters including localized clamping and specific clamping forces. A range of clamping forces was investigated—in some instances, the clamping force was insufficient to prevent leaks, and in other cases the clamping force was so great that flow was significantly reduced or even stopped entirely in some or all microchannels. Without being bound by theory, it was though that the was due to the channel cross section being deformed by the clamping force, reducing the cross-sectional area and making the channels more vulnerable to blockages due, for example, either to dust or the tissue occupying the entire microchannel.

Microfluid chips, in some embodiments, are fabricated from polydimethylsiloxane (PDMS). Other substrates may be used.

### Samples

In some embodiments, a sample is a biological sample. Non-limiting examples of biological samples include tissues, cells, and bodily fluids (e.g., blood, urine, saliva, cerebrospinal fluid, and semen). The biological sample may be adult tissue, embryonic tissue, or fetal tissue, for example. In some embodiments, a biological sample is from a human or other animal. For example, a biological sample may be obtained from a murine (e.g., mouse or rat), feline (e.g., cat), canine (e.g., dog), equine (e.g., horse), bovine (e.g., cow), leporine (e.g., rabbit), porcine (e.g., pig), hircine (e.g., goat), ursine (e.g., bear), or piscine (e.g., fish). Other animals are contemplated herein.

In some embodiments, a biological sample is fixed, and thus is referred to as a fixed biological sample. Fixation (e.g., tissue fixation) refers to the process of chemically preserving the natural state of a biological sample, for example, for subsequent histological analysis. Various fixation agents are routinely used, including, for example, formalin (e.g., formalin fixed paraffin embedded (FFPE) tissue), formaldehyde, paraformaldehyde and glutaraldehyde, any of which may be used herein to fix a biological sample. Other fixation reagents (fixatives) are contemplated herein.

In some embodiments, the biological sample is a tissue. In some embodiments, the biological sample is a cell. A biological sample, such as a tissue or a cell, in some embodiments, is sectioned and mounted on a surface, such as a slide. In such embodiments, the sample may be fixed before or after it is sectioned. In some embodiments, the fixation process involves perfusion of the animal from which the sample is collected.

### Kits

Also provided herein are kits for producing a high resolution spatial epigenomic map of a biological sample, for example. In some embodiments, the kits comprise a ligation linker sequence, a first set of barcoded polynucleotides, and a second set of barcoded polynucleotides.

In some embodiments, the kits comprise a (i) a primary antibody that specifically binds to an epigenomic marker of interest, (ii) a secondary antibody and (iii) a protein A tethered transposase. In one embodiment the protein A tethered transposon is preloaded with a ligation adaptor sequence.

In some embodiments, the kits comprise at least one reagent selected from tissue fixation reagents, reverse transcription reagents, ligation reagents, polymerase chain reaction reagents, template switching reagents, and sequencing reagents.

In some embodiments, the kits comprise tissue slides (e.g., glass slides).

In some embodiments, the kits comprise at least one microfluidic chip that comprises parallel or serpentine microchannels.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Spatial Epigenome-Transcriptome Co-Profiling of Mammalian Tissues

Emerging spatial technologies including spatial transcriptomics and spatial epigenomics are becoming powerful tools for profiling cellular states in the tissue context (Liu, 2021, Cell, 183:1665-1681; Deng, 2022, Science, 375:681-686; Deng, 2022, Nature, 609:375-383; Chen, 2022, Cell, 185:1777-1792; Lu, 2022, Cell, 185:4448-4464). However, current methods capture only one layer of omics information at a time, precluding the possibility to examine the mechanistic relationship across the central dogma of molecular biology. Disclosed herein are technologies for spatially resolved genome-wide joint profiling of epigenome and transcriptome by co-sequencing chromatin accessibility and gene expression (spatial-ATAC-RNA-seq) or histone modifications (H3K27me3, H3K27ac or H3K4me3) and gene expression (spatial-CUT&Tag-RNA-seq) on the same tissue section at near single-cell resolution. Additionally disclosed are the application of these technologies to embryonic and juvenile mouse brain as well as adult human brain to map how epigenetic mechanisms control transcriptional phenotype and cell dynamics in tissue. Although high concordant tissue features were identified by either spatial epigenome or spatial transcriptome, distinct patterns, suggesting their differential roles in defining cell states were also observed. These technologies link epigenome to transcriptome pixel-by-pixel and enable new discoveries in spatial epigenetic priming, differentiation, and gene regulation within the tissue architecture. The disclosed technologies are directly applicable to life science and biomedical research.

Herein, to further investigate the epigenetic mechanism underlying gene expression regulation, technologies for spatially resolved genome-wide co-mapping of epigenome and transcriptome by simultaneously profiling chromatin accessibility and mRNA expression (spatial-ATAC-RNA-seq) or histone modifications and mRNA expression (spatial-CUT&Tag-RNA-seq, H3K27me3, H3K27ac, or H3K4me3 histone modifications, respectively) on the same tissue section at the cellular level via deterministic co-barcoding to integrate the chemistry of spatial-ATAC-seq (Deng, 2022, Nature, 609:375-383) or spatial-CUT&Tag (Deng, 2022, Science, 375:681-686) with that for spatial transcriptomics, are disclosed. Also disclosed are the application of these technologies to embryonic and juvenile mouse brains as well as adult human brain hippocampus to dissect epigenetic states in regulating cell types and dynamics in tissue.

The materials and methods employed in these experiments are now described.

### Preparation of Tissue Slides

Mouse C57 Embryo Sagittal Frozen Sections (MF-104-13-C57) were purchased from Zyagen (San Diego, CA). The freshly harvested E13 mouse embryos were snap-frozen in OCT blocks and sectioned with 7-10 μm thickness. The tissue sections were collected on poly-L-lysine coated glass slides (Electron Microscopy Sciences, 63478-AS).

Juvenile mouse brain tissue (P21-P22) was obtained from Sox10:Cre-RCE:LoxP (EGFP) line on a C57BL/6xCD1 mixed genetic background maintained at Karolinska Institutet. The line was first generated by crossing Sox10:Cre animals (The Jackson Laboratory, 025807) with RCE: loxP (enhanced green fluorescent protein (EGFP)) animals (The Jackson Laboratory, 32037-JAX). It was established and maintained by breeding males without the Cre allele with females carrying a hemizygous Cre allele, while the reporter allele was kept in homozygosity or hemizygosity in both males and females. This results in specific labelling of oligodendrocyte lineage with EGFP. All animals were free from mouse bacterial and viral pathogens, ectoparasites and endoparasites. The following light/dark cycle was kept for the mice: dawn 6:00-7:00, daylight 7:00-18:00, dusk 18:00-19:00, night 19:00-6:00. Mice were housed in individually ventilated cages with a maximum number of 5 per cage (IVC sealsafe GM500, tecniplast). General housing parameters such as temperature, ventilation, and relative humidity followed the European convention for the protection of vertebrate animals used for experimental and other scientific purposes. The air quality was controlled by using the stand-alone air handling units equipped with a HEPA filter. The consistent relative air humidity was 55%+10 with a temperature of 22° C. The husbandry parameters were monitored with ScanClime® (Scanbur) units. The cages contained card box shelter, gnawing sticks, and nesting material (Scanbur), placed on a hardwood bedding (TAPVEI, Estonia). The mice were provided a regular chow diet and water was supplied with a water bottle and changed weekly. Cages were changed every two weeks in a laminar air-flow cabinet.

Mice were sacrificed at P21/P22 (both sexes were used) by anesthesia with ketamine (120 mg/kg of body weight) and xylazine (14 mg/kg of body weight), followed by transcranial perfusion with cold oxygenated artificial cerebrospinal fluid aCSF (87 mM NaCl, 2.5 mM KCl, 1.25 mM NaH2PO4, 26 mM NaHCO3, 75 mM Sucrose, 20 mM Glucose, 1 mM CaCl2*2H2O and 2 mM MgSO4*7H2O in dH2O). Upon isolation, the brains were kept for a minimal time period in aCSF until embedding in Tissue-Tek® O.C.T. compound (Sakura) and snap-freezing using a mixture of dry ice and ethanol. Coronal cryosections of 10 μm were mounted on poly-L-lysine coated glass slides (63478-AS, Electron Microscopy Sciences or 2″×3″ glass slides, AtlasXomics).

All experimental procedures were conducted following the European directive 2010/63/EU, local Swedish directive L150/SJVFS/2019:9, Saknr L150, and Karolinska Institutet complementary guidelines for procurement and use of laboratory animals, Dnr. 1937/03-640. All the procedures described were approved by the local committee for ethical experiments on laboratory animals in Sweden (Stockholms Norra Djurförsöksetiska nämnd), lic. nr. 1995/2019 and 7029/2020.

The human brain tissue was obtained from the Brain Collection of the New York State Psychiatric Institute (NYSPI) at Columbia University, which includes brain samples from the Republic of Macedonia. Brain tissue collection was conducted with NYSPI Institutional Review Board approval and informed consent obtained from next of kin who agreed to donate the brains and participated in psychological autopsy interviews.

Analysis was performed on brain hippocampus tissue from a 31 year-old Caucasian male, with postmortem interval (PMI, time from demise to brain collection) of 6.5 hours, with no psychiatric or neurological diagnosis, who died of a traumatic accident, and had a high global functioning before death as measured by Global Assessment Scale (GAS) (Endicott, 1976, General Psychiatry, 33:766-771) score which was 90 (score 1 to 100, with 100 the highest functioning), and with toxicology negative for psychotropic medications and drugs.

The anterior hippocampal region was dissected from a fresh frozen coronal section (20 mm thickness) of the right brain hemisphere. The dentate gyrus region (around 10 mm×10 mm) of the anterior hippocampal region was selected. The cryosections of 10 μm were collected on poly-L-lysine coated glass slides (63478-AS, Electron Microscopy Sciences). The samples were stored at −80° C. until further use.

### Preparation of Transposome

Unloaded Tn5 transposase (C01070010) and pA-Tn5 (C01070002) were purchased from Diagenode, and the transposome was assembled following manufacturer's guidelines. The oligos applied for transposome assembly were:

Tn5ME-A,
(SEQ ID NO: 214)
5′-TCGTCGGCAGCGTCAGATGTGTATAAGAGACAG-3′;
Tn5MErev,
(SEQ ID NO: 215)
5′-/5Phos/CTGTCTCTTATACACATCT-3′;
Tn5ME-B,
(SEQ ID NO: 216)
5′-/5Phos/CATCGGCGTACGACTAGATGTGTATAAGAGACAG-3′.

### DNA Barcodes Sequences, DNA Oligos, and Other Key Reagents

DNA oligos used for PCR and library construction were shown in Table 5, All the DNA barcodes sequences were provided in Table 6, Table 7, and all other chemicals and reagents were listed in Table 8.

### Fabrication of the PDMS Microfluidic Device

The chrome photomasks were purchased from Front Range Photomasks (Lake Havasu City, AZ) with the channel width of 20 μm or 50 μm. The molds for polydimethylsiloxane (PDMS) microfluidic devices were fabricated with standard photolithography. The manufacturer's guidelines were followed to spin-coat SU-8-negative photoresist (SU-2025, SU-2010, Microchem) onto a silicon wafer (C04004, WaferPro). The heights of the features were about 20 μm and 50 μm for 20-μm-wide and 50-μm-wide devices, respectively. The PDMS microfluidic devices were fabricated via the SU-8 molds. The curing and base agent were mixed at a 1:10 ratio and poured it into the molds. After degassing for 30 minutes, the mixture was cured at 70° C. for 2 hours. The solidified PDMS was cut out for further use. A detailed protocol for the fabrication and preparation of the PDMS device52 was published.

### Spatial-ATAC-RNA-Seq:

The frozen tissue slide was thawed for 10 minutes at room temperature. The tissue was fixed with formaldehyde (0.2%, with 0.05 U/μL RNase Inhibitor) for 5 minutes and quenched with 1.25 M glycine for another 5 minutes. After fixation, the tissue was washed with 1 mL 0.5×DPBS-RI twice and cleaned with DI water.

The tissue permeabilization was done with 200 μL lysis buffer (3 mM MgCl2; 0.01% Tween-20; 10 mM Tris-HCl, pH 7.4; 0.01% NP-40; 10 mM NaCl; 1% BSA; 0.001% Digitonin; 0.05 U/μL RNase Inhibitor) for 15 minutes and was washed by 200 μL wash buffer (10 mM Tris-HCl pH 7.4; 10 mM NaCl; 3 mM MgCl2; 1% BSA; 0.1% Tween-20) twice for 5 minutes. Transposition mix (5 μL home-made transposome; 33 μL 1×DPBS; 50 μL 2× Tagmentation buffer; 1 μL 1% Digitonin; 1 μL 10% Tween-20; 0.05 U/μL RNase Inhibitor; 10 μL Nuclease-free H2O) was added and incubated at 37° C. for 30 minutes. After that, 200 μL 40 mM EDTA with 0.05 U/μL RNase Inhibitor was added and incubated for 5 minutes at room temperature to stop transposition. Finally, the tissue section was washed with 200 μL 0.5×PBS-RI twice for 5 minutes and cleaned with DI water.

For reverse transcription, RT mixture (12.5 μL 5×RT Buffer; 4.5 μL RNase-free water; 0.4 μL RNase Inhibitor; 0.8 μL SuperaseIn RNase Inhibitor; 3.1 μL 10 mM dNTPs each; 6.2 L Maxima H Minus Reverse Transcriptase; 25 μL 0.5×PBS-RI; 10 μL RT primer) was added. The tissue was incubated 30 minutes at room temperature, and 42° C. for 90 minutes in a wet box. After the RT reaction, the tissue was washed with 1×NEBuffer 3.1 with 1% RNase Inhibitor for 5 min.

For 1st barcodes (barcodes A) in situ ligation, the 1st PDMS chip was covered to the region of interest (ROI) on the tissue. For alignment purpose, 10× objective (Thermo Fisher Scientific, EVOS FL Auto microscope (AMF7000), EVOS FL Auto 2 Software Revision 2.0.2094.0) was used to take a brightfield image. The PDMS device and tissue slide were clamped with a home-made acrylic clamp tightly. At first, barcode A was annealed with ligation linker 1, 10 μL 100 μM ligation linker, 10 μL 100 μM each barcode A, and 20 μL 2× annealing buffer (20 mM Tris; pH 7.5-8.0; 100 mM NaCl; 2 mM EDTA) were mixed well. For each channel, 5 μL ligation master mixture was prepared with 2 μL of ligation mix (27 μL of T4 DNA ligase buffer; 72.4 μL of RNase free water; 5.4 μL of 5% Triton X-100; 11 μL T4 DNA ligase), 2 μL of 1× NEBuffer 3.1, and 1 μL of each annealed DNA barcode A (A1-A50/A100, 25 μM). Vacuum was used to load ligation master mixture into 50 channels of the device. The device was incubated at 37° C. for 30 minutes in a wet box. The PDMS chip and clamp were removed after washed with 1×NEBuffer 3.1 for 5 minutes. The slide was washed with water and dried by air.

For 2nd barcodes (barcodes B) in situ ligation, the 2nd PDMS chip was covered to the slide. Another brightfield image with 10× objective was taken. The acrylic clamp was applied to clamp the PDMS and tissue slide together. The annealing of barcodes B (B1-B50/B100, 25 μM) and the preparation of ligation master mix were the same with barcodes A. The device was incubated at 37° C. for 30 minutes in a wet box. The PDMS chip and clamp were removed after washing with 1×DPBS with SUPERase In RNase Inhibitor for 5 minutes. The slide was washed with water and dried by air. The brightfield image was taken for further alignment.

For lysis of the tissue, the ROI of the tissue was digested with 100 μL reverse crosslinking mixture (0.4 mg/mL proteinase K; 1 mM EDTA; 50 mM Tris-HCl, pH 8.0; 200 mM NaCl; 1% SDS) at 58° C. for 2 hours in a wet box. The lysate was collected into a 1.5 mL tube and incubated at 65° C. overnight.

For DNA and cDNA separation, the lysate was purified with Zymo DNA Clean & Concentrator-5 and eluted to 100 μL of RNase free water. The 1× B&W buffer with 0.05% Tween-20 was used to wash 40 μL Dynabeads MyOne Streptavidin C1 beads for 3 times. Then 200 μL 2× B&W buffer with 2.5 μL of SUPERase In RNase Inhibitor was used to resuspend the beads. The resuspended beads were mixed with the lysate and allowed binding at room temperature for 1 hour with agitation. A magnet was used to separate beads and supernatant in the lysate.

The supernatant was taken out for ATAC library construction. The supernatant was purified with Zymo DNA Clean & Concentrator-5 and eluted to 20 μL of RNase free water. PCR solution (25 μL 2×NEBNext Master Mix; 2.5 μL 25 μM indexed i7 primer; 2.5 μL 25 μM P5 PCR primer) was added and mix well. The PCR was first conducted by following the program: 72° C. 5 minutes, 98° C. 30 seconds, and cycled at 98° C. 10 seconds, 63° C. 30 seconds, and 72° C. 1 minute for 5 times. To figure out additional cycles, the pre-amplified mixture (5 μL) was mixed with qPCR solution (5 μL 2×NEBNext Master Mix; 0.24 μL 25×SYBR Green; 0.5 μL 25 μM new P5 PCR primer; 3.76 μL nuclease-free H2O; 0.5 L 25 μM indexed i7 primer). Then, qPCR reaction was conducted as following program: 98° C. 30 seconds, and cycled at 98° C. 10 seconds, 63° C. 30 seconds, and 72° C. 1 minute for 20 times. The remaining pre-amplified DNA (45 μL) was amplified by running additional cycles determined by qPCR (reach ⅓ of saturated signal). The final PCR product was purified by 1× Ampure XP beads (45 μL) and eluted in 20 μL nuclease-free H2O.

The beads were used for cDNA library construction. The beads were washed twice with 400 μL 1× B&W buffer with 0.05% Tween-20 and once more with 10 mM Tris (pH 8.0) containing 0.1% Tween-20. Streptavidin beads with bound cDNA molecules were resuspended in a TSO solution (22 μL 10 mM dNTPs each; 44 μL 5× Maxima RT buffer; 44 μL 20% Ficoll PM-400 solution; 88 μL RNase free water; 5.5 L of 100 μM template switch primer; 11 μL of Maxima H Minus Reverse Transcriptase; 5.5 μL of RNase Inhibitor). The beads were incubated at room temperature for 30 minutes and then at 42° C. for 90 minutes with gentle shaking. After washing beads once with 400 μL 10 mM Tris with 0.1% Tween-20 and once with water, the beads were resuspended into a PCR solution (110 μL 2× Kapa HiFi HotStart Master Mix; 8.8 μL 10 μM primer 1 and primer 2; 92.4 μL RNase free water). The PCR thermocycling was performed as following program: 95° C. 3 minutes, and cycled at 98° C. 20 seconds, 65° C. 45 seconds, 72° C. 3 minutes for 5 times. After five cycles, Dynabeads MyOne Streptavidin C1 beads were removed from PCR solution and 25×SYBR Green was added at 1× concentration. Samples were again placed in a qPCR machine with the following thermocycling conditions: 95° C. for 3 minutes, cycled at 98° C. 20 seconds, 65° C. 20 seconds, and 72° C. 3 minutes for 15 times, followed by a single 5 minutes at 72° C. after cycling. The reaction was removed once qPCR signal began to plateau. The PCR product was purified with a 0.8× Ampure XP beads and eluted in 20 μL nuclease-free H₂O.

A Nextera XT Library Prep Kit was used for library preparation. The purified cDNA (1 ng) was diluted in RNase free water to a total volume of 5 μL. 10 μL Tagment DNA buffer and 5 μL Amplicon Tagment mix was added and incubated at 55° C. for 5 minutes. 5 μL of NT buffer was added and incubated at room temperature for 5 minutes. PCR master solution (15 μL PCR master mix; 1 μL 10 μM P5 primer; 1 μL 10 μM indexed P7 primer; 8 μL RNase free water) was added and PCR reaction was performed with the following program: 95° C. 30 seconds, cycled at 95° C. 10 seconds, 55° C. 30 seconds, 72° C. 30 seconds, and 72° C. for 5 minutes after 12 cycles. The PCR product was purified with a 0.7× Ampure XP beads to get the library.

The Agilent Bioanalyzer High Sensitivity Chip was used to get the size distribution and concentration of the library before sequencing. Next Generation Sequencing (NGS) was conducted on Illumina NovaSeq 6000 sequencer (pair-end 150 bp mode).

### Spatial-CUT&Tag-RNA-Seq:

The frozen tissue slide was thawed for 10 minutes at room temperature. The tissue was fixed with formaldehyde (0.2%, with 0.05 U/μL RNase Inhibitor) for 5 minutes and quenched with 1.25M glycine for another 5 minutes. After fixation, the tissue was washed with 1 mL wash buffer (150 mM NaCl; 20 mM HEPES pH 7.5; 1 tablet Protease inhibitor cocktail; 0.5 mM Spermidine) twice and dip into DI water. The tissue section was permeabilized with NP40-Digitonin wash buffer (0.01% Digitonin; 0.01% NP40; in wash buffer) for 5 minutes. The primary antibody (1:50 dilution with antibody buffer (0.001% BSA; 2 mM EDTA; in NP40-Digitonin wash buffer) was added and incubated at 4° C. overnight. The secondary antibody (Guinea Pig anti-Rabbit IgG) (1:50 dilution with NP40-Digitonin wash buffer) was added and incubated 30 minutes at room temperature. The tissue was washed with wash buffer for 5 minutes. A 1:100 dilution of pA-Tn5 adapter complex in 300-wash buffer (1 tablet Protease inhibitor cocktail; 300 mM NaCl; 0.5 mM Spermidine; 20 mM HEPES pH 7.5) was added and incubated at room temperature for 1 hour. Then followed with a 5 minute wash with 300-wash buffer. Tagmentation buffer (10 mM MgCl2 in 300-wash buffer) was added and incubated at 37° C. 1 hour. Then, 40 mM EDTA with 0.05 U/μL RNase Inhibitor was added and incubated at room temperature for 5 minutes to stop tagmentation. The tissue was washed twice with 0.5×DPBS-RI for 5 minutes for further use.

For reverse transcription, two ligations, and beads separation, the protocols were the same with Spatial-ATAC-RNA-seq.

For the construction of CUT&Tag library, the supernatant was purified with Zymo DNA Clean & Concentrator-5 and eluted to 20 μL of RNase free water. PCR solution (2 μL 10 μM P5 PCR primer and indexed i7 primer; 25 μL NEBNext Master Mix) was added and mixed well. The PCR was performed with the program: 58° C. 5 minutes, incubated at 72° C. 5 minutes and 98° C. 30 seconds, then cycled at 98° C. 10 seconds, and incubated at 60° C. 10 seconds for 12 times, and final incubated at 72° C. 1 minutes. The PCR product was purified by 1.3× Ampure XP beads using the standard protocol and eluted in 20 μL nuclease-free H2O.

The cDNA libraries construction followed the former spatial-ATAC-RNA-seq protocol.

The Agilent Bioanalyzer High Sensitivity Chip was used to get the size distribution and concentration of the library before sequencing. Next-generation sequencing (NGS) was conducted on Illumina NovaSeq 6000 sequencer (pair-end 150 bp mode).

### Data Preprocessing

For ATAC and CUT&Tag data, linker 1 and linker 2 were used to filter Read 2, and the sequences were converted to Cell Ranger ATAC v1.2 format (10× Genomics). The genome sequences were in the newly formed Read 1, barcodes A and barcodes B were included in newly formed Read 2. Human reference (GRCh38) or mouse reference (GRCm38) was used to align the fastq files. The achieved BED like fragments were used to conduct downstream analysis. The fragments file includes fragments of information on spatial locations (barcode A×barcode B) and the genome.

For RNA data, the Read 2 was refined to extract Barcode A, Barcode B, and UMI. ST pipeline v1.7.2 (Navarro, 2017, Bioinformatics, 33:2591-2593) was used to map the processed read 1 against the mouse genome (GRCm38) or human genome (GRCh38), which created the gene matrix for downstream analysis. The gene matrix contains information of genes and spatial locations (barcode A×barcode B).

### Data Clustering and Visualization

The location of pixels on the tissue from the bright field image was first identified using MATLAB 2020b.

Library Signac v1.8 (Stuart, 2021, Nat Methods, 18:1333-1341) in R v4.1. The ATAC, CUT&Tag, and RNA matrices were read into Signac v1.8 (Stuart, 2021, Nat Methods, 18:1333-1341). The “DefaultAssay” function was used for the RNA assay. For RNA data visualization, the feature was set to 3000 with the “FindVariableFeatures” function. Then the data was normalized using the “SCTransform” function. Normalized RNA data was clustered, and RNA UMAP was built. The “DefaultAssay” function was applied to the ATAC/CUT&Tag assay. For ATAC/CUT&Tag data visualization, the min.cutoff was set with the “FindTopFeatures” function. The data was normalized and dimensionally reduced using Latent Semantic Indexing (LSI). Then the ATAC/CUT&Tag data was clustered and ATAC/CUT&Tag UMAP was built.

The “DefaultAssay” function was used for the joint ATAC/CUT&Tag and RNA assay. For visualization of joint ATAC/CUT&Tag and RNA data (Hao, 2021, Cell, 184:3573-3587), the “FindMultiModalNeighbors” function was used. The reduction.list was set to (“pca”, “Isi”), the dims.list was set to the same with RNA and ATAC/CUT&Tag, the modality.weight.name was set to RNA.weight, and the joint UMAP was built.

To plot the above generated UMAPs together, “DefaultAssay” was set to RNA, the UMAPs for ATAC/CUT&Tag, RNA, or joint ATAC/CUT&Tag and RNA were visualized separately using “DimPlot”.

As for RNA spatial data visualization, the gene matrix obtained from RNA was loaded into Seurat v4.1 (Hao, 2021, Cell, 184:3573-3587) as a Seurat object, the metadata of RNA obtained from Signac was read into the Seurat object. All the spatial maps were then plotted with the “SpatialPlot” function.

As for ATAC/CUT&Tag spatial data visualization, the fragment file got from ATAC/CUT&Tag was read into ArchR v1.0.1 (Granja, 2021, Nat Genet, 53:403-411) as an ArchRProject, the metadata of ATAC/CUT&Tag got from Signac was read into the ArchRProject. The data from ArchRProject was normalized and dimensionally reduced using iterative Latent Semantic Indexing (LSI). For GAS (gene activity score) and CSS (chromatin silencing score) calculation, using the Gene Score model in ArchR. A gene score matrix was obtained for downstream analysis. The “getMarkerFeatures” and “getMarkers” functions in ArchR (testMethod=“Wilcoxon”, cutOff=“FDR<=0.05”, groupBy=“seurat_cluster”) were used to find the marker genes/regions for each cluster. To visualize the spatial data, results obtained from ArchR were imputed to Seurat v4.1 to map the data back to the tissue. The size of the pixels were scaled using the “pt.size.factor” parameter in the Seurat package for better visualization.

For peak-to-gene links, RNA Seurat object was inputted using the “addGeneIntegrationMatrix” function in ArchR, then the peak-to-gene links were drawn with the “addPeak2GeneLinks” function. Co-accessibility of peaks was calculated using the “addCoAccessibility” function in ArchR.

### Integrative Data Analysis and Cell Type Identification

Seurat v4.1 (Hao, 2021, Cell, 184:3573-3587) was used for RNA data integration and cell type identification, the “SCTransform” function was used to normalize spatial RNA and scRNA-seq data. The “SelectIntegrationFeatures” function was used to get the common features of the two datasets. The “FindIntegrationAnchors” function was applied to find anchors. The “IntegrateData” function was used to get an integrated dataset through the identified anchors. The obtained integrated dataset was clustered, which showed a good match between spatial RNA and scRNA-seq data. The “FindTransferAnchors” function was used to find transfer anchors, and the transfer anchors were used to conduct label transfer with the “TransferData” function (if more than one cell type was presented in one pixel, the major cell type was assigned).

Signac v1.8 and Seurat v4.1 were used for the integration of ATAC/CUT&Tag and scATAC-seq/scCUT&Tag data. The scATAC-seq/scCUT&Tag data was quantified according to ATAC/CUT&Tag data to ensure that there were common features across two datasets. The “FindIntegrationAnchors” function (reduction= “rlsi”) was used to identify the anchors between two datasets. The “IntegrateEmbeddings” function was used to get an integrated dataset through the identified anchors. The obtained integrated dataset was clustered, which showed well match between spatial ATAC/CUT&Tag and scATAC-seq/scCUT&Tag data. For ATAC data, the “FindTransferAnchors” function was used to find transfer anchors, and the transfer anchors were used to map scATAC-seq to spatial ATAC data with the “MapQuery” function.

ArchR v1.0.1 was used for cell type identification for ATAC/CUT&Tag data from scRNA-seq data. The gene score matrix of ATAC/CUT&Tag was compared with the gene expression matrix from scRNA-seq and aligned pixels from ATAC/CUT&Tag data with cells from scRNA-seq. The “GeneIntegrationMatrix” was used to add Pseudo-scRNA-seq profiles and cell identities.

### Correlation of CSS/GAS and Gene Expression

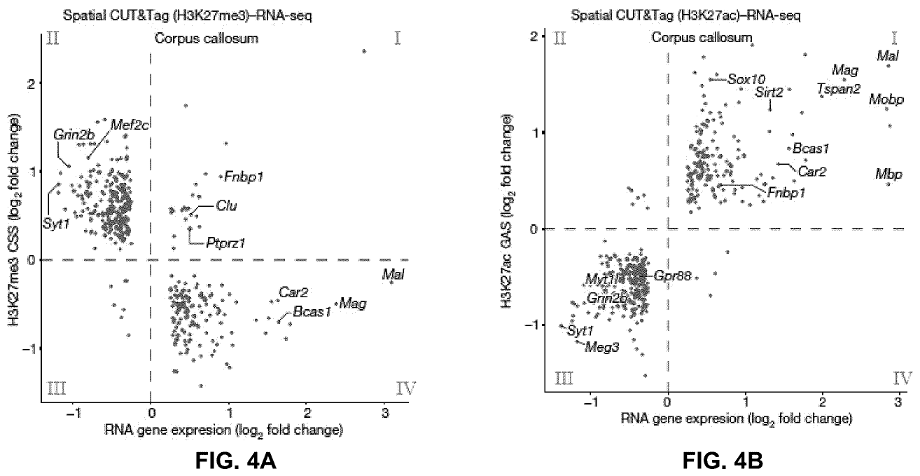

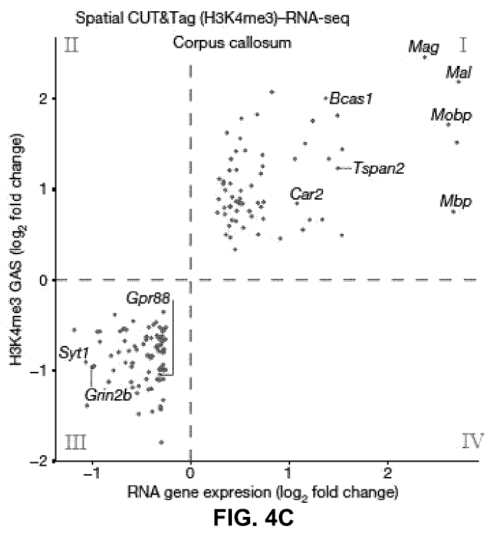

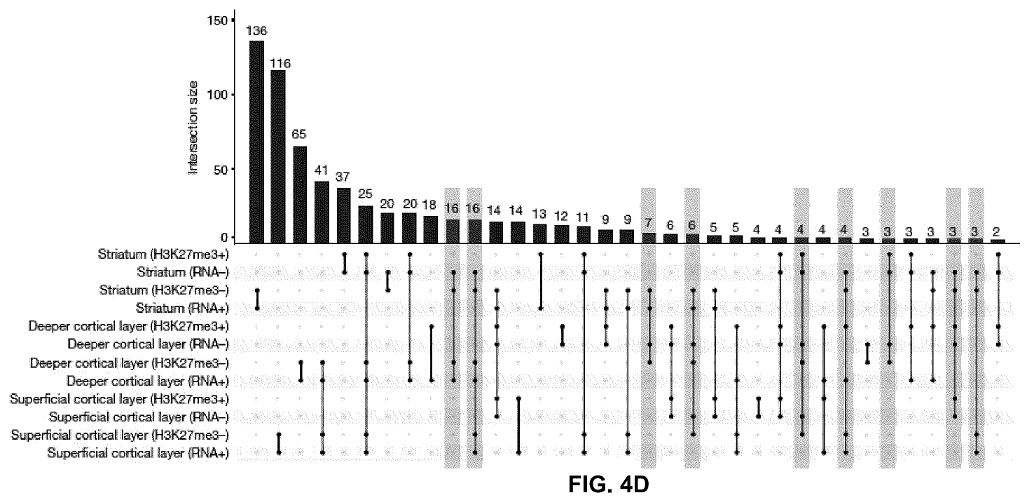

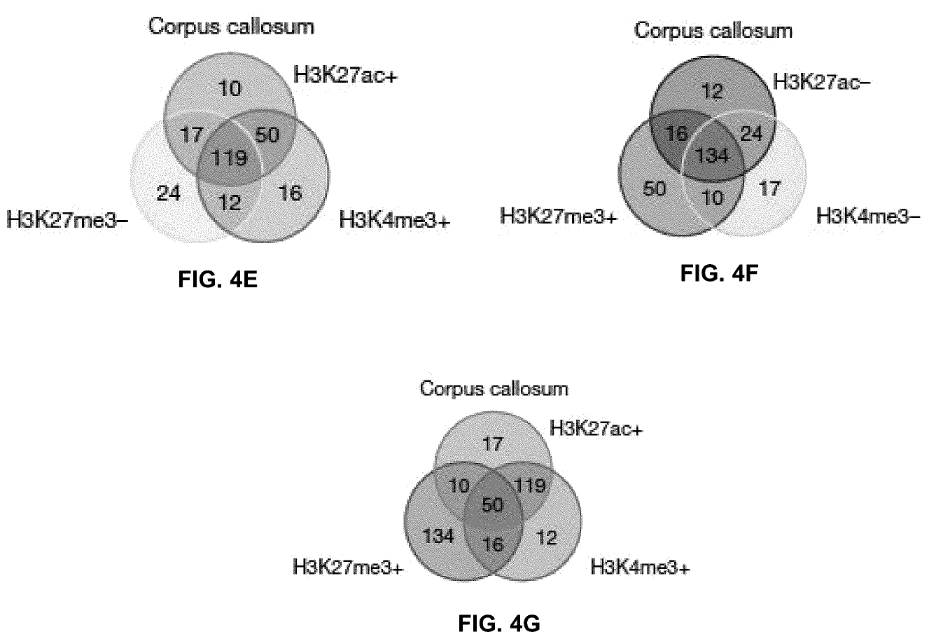

Correlation analysis was done by different tissue regions. The mouse brain hemisphere was separated into 7 clusters (corpus callosum, striatum, superficial cortical layer, deeper cortical layer, lateral ventricle, lateral septal nucleus, and others) according to the RNA clusters and anatomical annotation, and named “tissue_clusters”. For FIG. 4A-FIG. 4C, the “FindMarkers” function (settings: min.pct=0.25, logfc.threshold=0.25) was used to calculate RNA data, the genes with p_val_adj<10-5 were selected as marker genes. The “getMarkerFeatures” function (settings: groupBy=“tissue_clusters”) was applied to calculate the GAS or CSS of genes from the marker gene list (cutOff=“FDR<=0.05” & (cutOff=“Log 2FC>=0.1” or cutOff=“Log 2FC<=−0.1”). If avg_log 2FC>0 (RNA) and Log 2FC>0 (CUT&Tag) for a specific gene, it showed in quadrant I. The GO enrichment analysis was conducted with the “enrichGO” function in clusterProfiler v4.2 package (Wu, 2021, Innovation (N Y), 2:100141).

### Technology Workflow and Data Quality

Spatial-ATAC-RNA-seq is shown schematically in FIG. 1A and FIG. 6A-FIG. 6C. A frozen tissue section was fixed with formaldehyde and treated with the Tn5 transposition complex pre-loaded with a DNA adapter containing a universal ligation linker that can be inserted into the transposase accessible genomic DNA loci. The same tissue section was then incubated with a biotinylated DNA adapter containing a universal ligation linker and a poly-T sequence that binds to the poly-A trail of mRNAs to initiate reverse transcription (RT) in tissue. Afterwards, a microfluidic channel array chip was placed on the tissue section to introduce spatial barcodes Ai (i=1-50 or 100) that were covalently linked to the universal ligation linker via templated ligation. Afterwards, another microchip with microchannels perpendicular to the first flow direction was used to introduce spatial barcodes Bj (j=1-50 or 100) that were ligated to barcodes Ai (j=1-50 or 100), resulting in a 2D grid of spatially barcoded tissue pixels defined by the unique combination of barcodes Ai and Bj (i=1-50 or 100, j=1-50 or 100, n of barcoded pixels=2,500 or 10,000). Finally, barcoded cDNA and genomic DNA (gDNA) fragments were released after reserve crosslinking. cDNAs were enriched with streptavidin beads and gDNA fragments were retained in the supernatant. The libraries of gDNA and cDNA were constructed separately for next-generation sequencing (NGS). Spatial-CUT&Tag-RNA-seq was realized by applying an antibody against specific histone modification (i.e., H3K27me3, H3K27ac, or H3K4me3) to the tissue section and then use protein A tethered Tn5-DNA complex to perform co-assay of cleavage under targets and tagmentation (CUT&Tag). The remaining steps are similar to spatial-ATAC-RNA-seq (see FIG. 1A and FIG. 6A-FIG. 6E) but resulted in spatial co-profiling of histone modification and transcriptome.

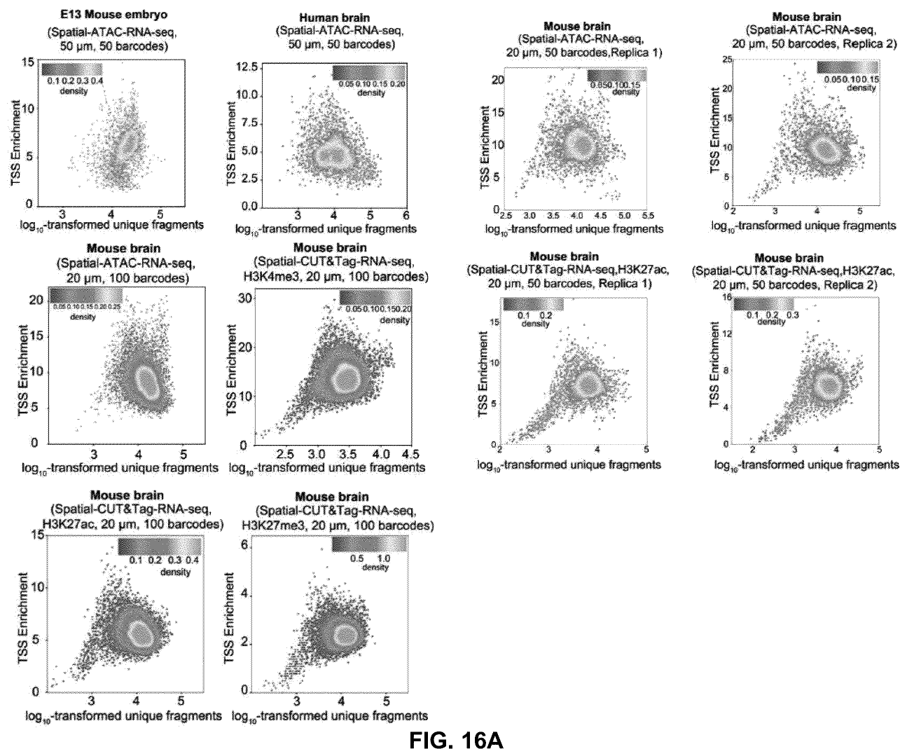

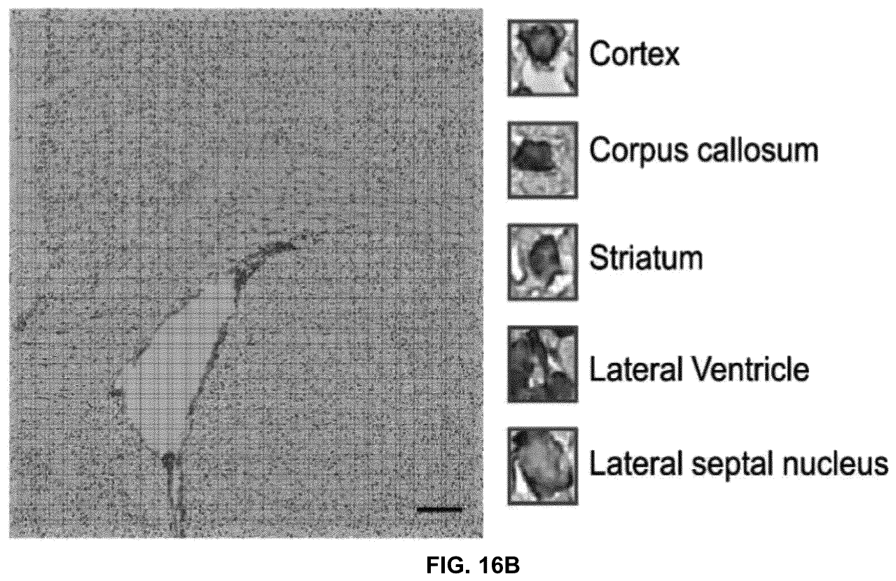

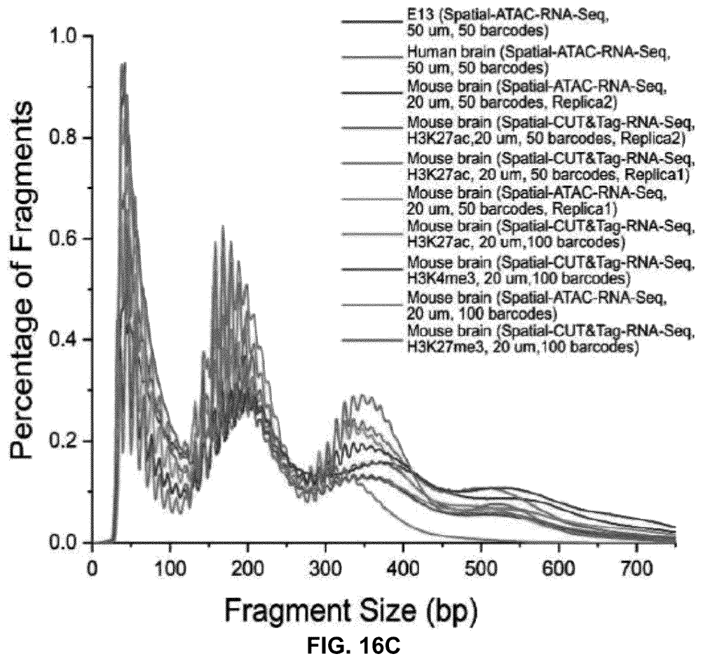

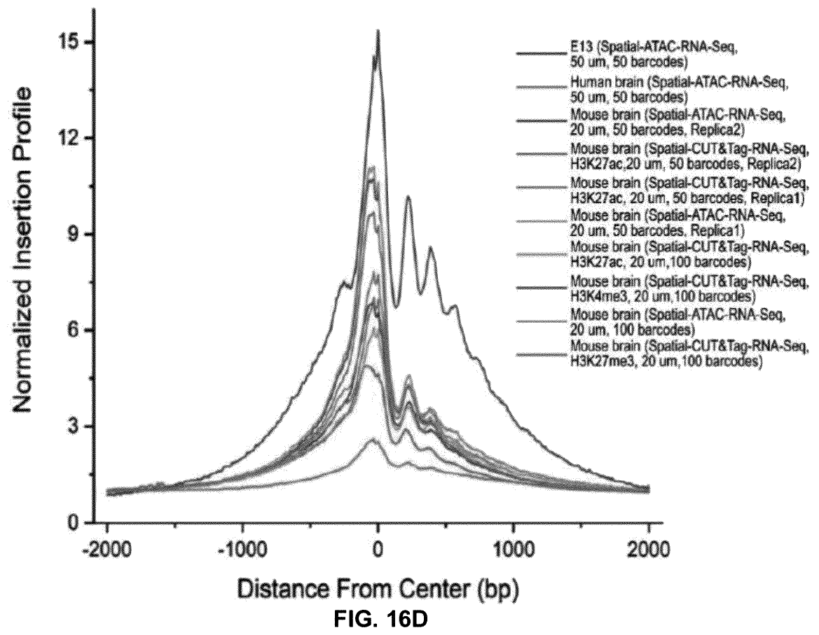

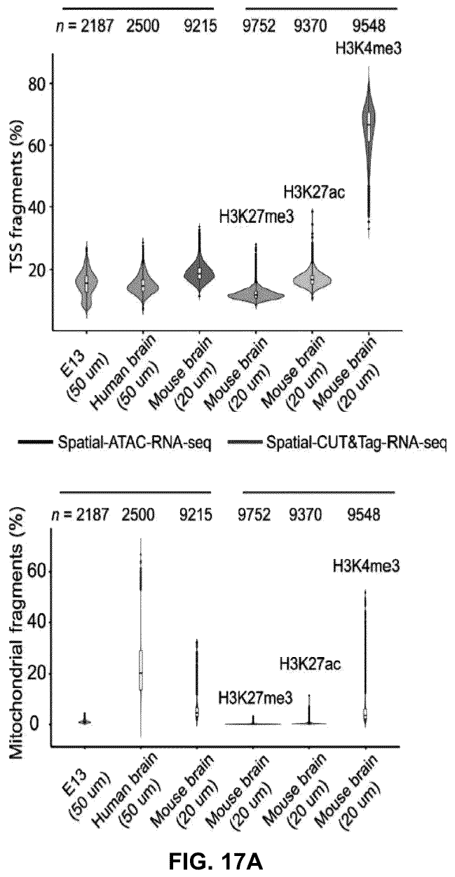

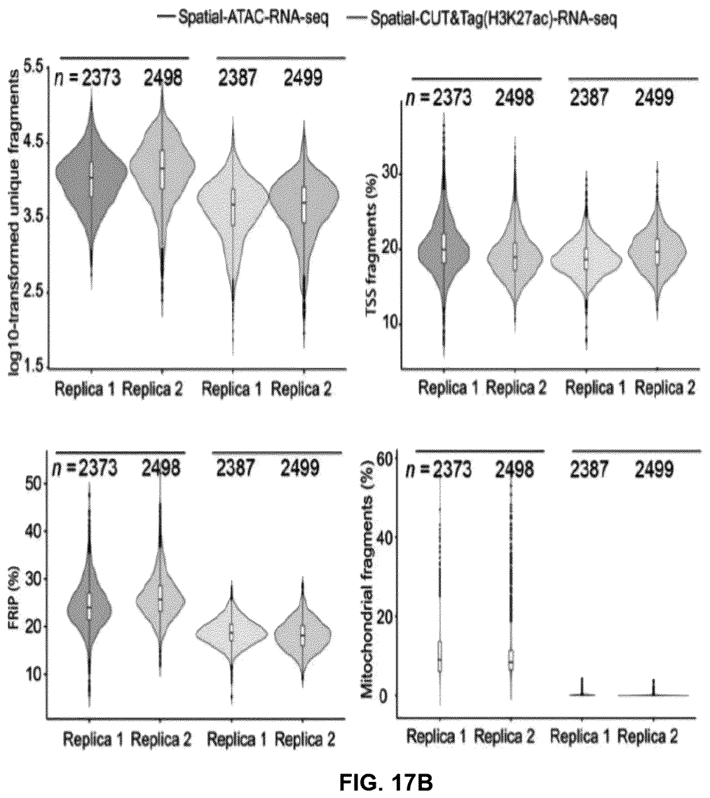

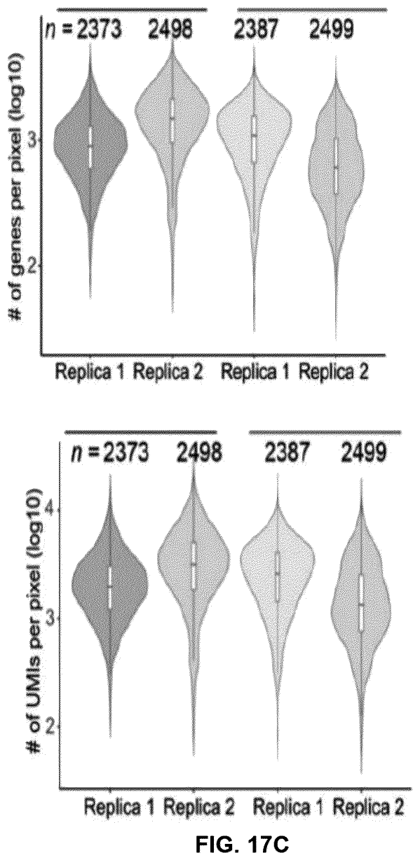

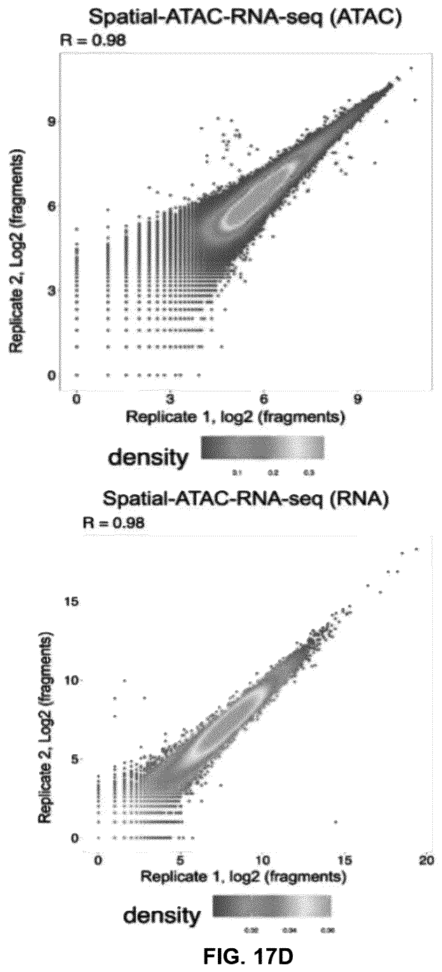

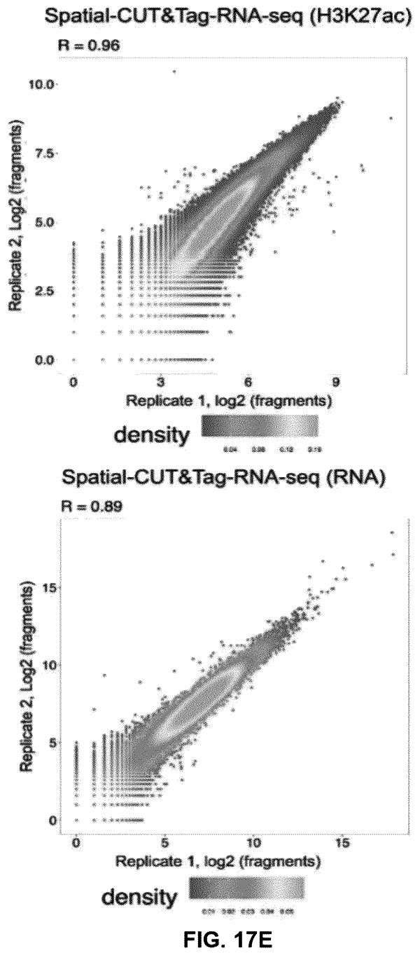

Spatial-ATAC-RNA-seq experiments were performed on the embryonic day 13 mouse embryo (E13) (pixel size 50 μm), mouse postnatal day 21/22 (P21/22) brains (pixel size 20 μm), and adult human brain hippocampus tissue (pixel size 50 μm). For 50 μm pixel size, an average of 25 cells for E10 mouse embryo (Liu, 2020, Cell, 183:1665-1681) and 1-9 cells for human hippocampus (Deng, 2022, Nature, 609:375-383) were obtained. Most of the 20 μm pixels contained 1-3 cells per pixel in juvenile mouse brain (FIG. 16B. Using a 100×100 barcodes scheme, the mapping area can cover nearly the entire hemisphere of a P22 mouse brain coronal section. A median of 14,284 unique fragments per pixel among which 19% were enriched in the TSS regions and 26% located in peaks was obtained (FIG. 1B, FIG. 16A, and FIG. 17A). For the RNA portion, totally 22,914 genes were detected with an average of 1,073 genes and 2,358 UMIs per pixel (spatial-ATAC-RNA-seq) (FIG. 1C).

Spatial-CUT&Tag-RNA-seq for H3K27me3, H3K27ac or H3K4me3 was performed on mouse postnatal day 21/22 (P21/22) brains (pixel size 20 μm). With 100×100 barcodes device, a median of 10,644 (H3K27me3), 10,002 (H3K27ac), and 2,507 (H3K4me3) unique fragments per pixel were obtained, of which 12% (H3K27me3), 17% (H3K27ac), and 67% (H3K4me3) of fragments overlapped with TSS regions, and 12% (H3K27me3), 21% (H3K27ac), and 54% (H3K4me3) were located in peaks (FIG. 1B, FIG. 16A, and FIG. 17A). For the RNA data, totally 25,881 (H3K27me3), 23,415 (H3K27ac), and 22,731 (H3K4me3) genes were detected with an average of 2,011 (H3K27me3), 1,513 (H3K27ac), and 1,329 (H3K4me3) genes per pixel or 4,734 (H3K27me3), 3,580 (H3K27ac), and 2,885 (H3K4me3) UMIs per pixel (FIG. 1C). The assessment of data quality for mouse embryo, P21 mouse brain, and human brain samples with 50×50 barcodes is also included in FIG. 1B, FIG. 1C, FIG. 16A, FIG. 17A-FIG. 17C, Table 3, and Table 4.

Moreover, the insert size distributions of chromatin accessibility (spatial-ATAC-RNA-seq) and histone modifications (spatial-CUT&Tag-RNA-seq, H3K27me3, H3K27ac, or H3K4me3) fragments were consistent with the captured nucleosomal fragments in all tissues (FIG. 16C-FIG. 16D). The correlation analysis between replicates showed high reproducibility (r=0.98 for ATAC, r=0.98 for RNA in spatial-ATAC-RNA-seq; r=0.96 for CUT&Tag (H3K27ac), r=0.89 for RNA in spatial-CUT&Tag-RNA-seq, r stands for Pearson correlation coefficient, FIG. 17D-FIG. 17E). Tissue type, preparation, and quality may influence analytical metrics (Methods).

### Spatial Co-Mapping of Mouse Embryo

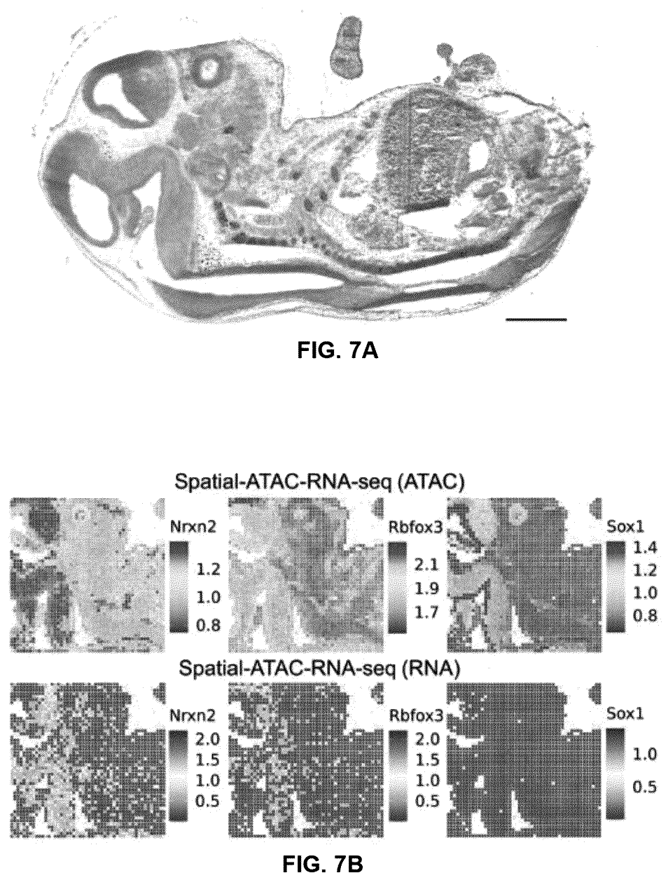

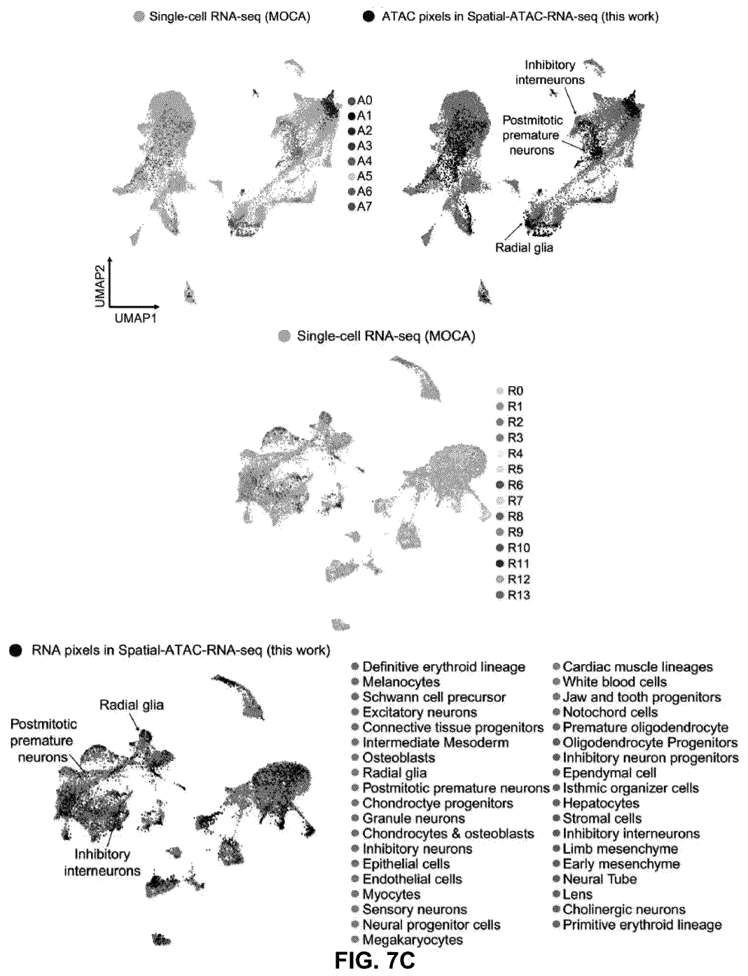

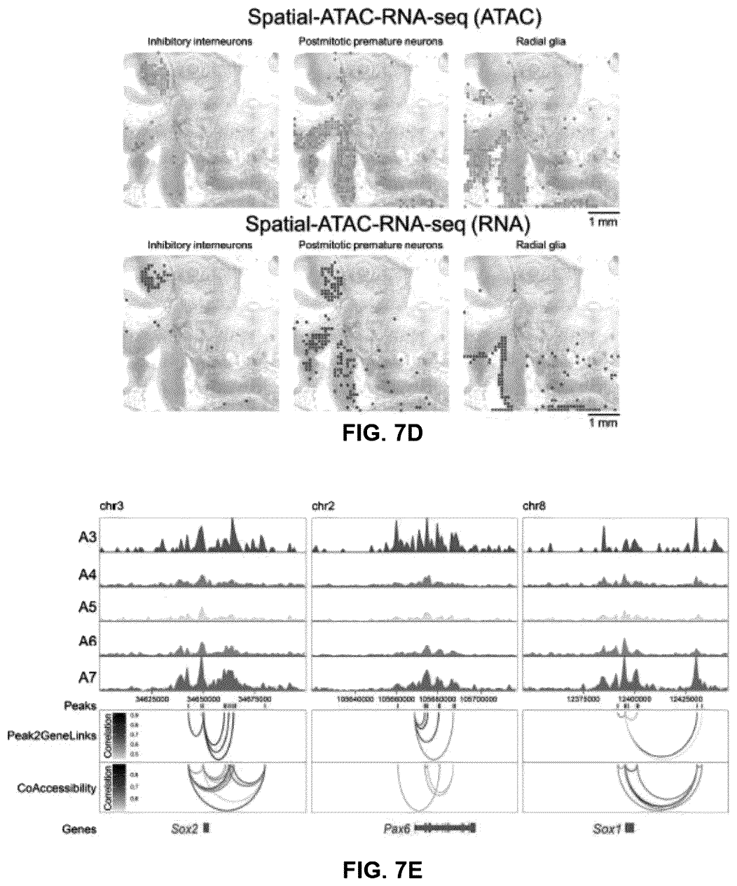

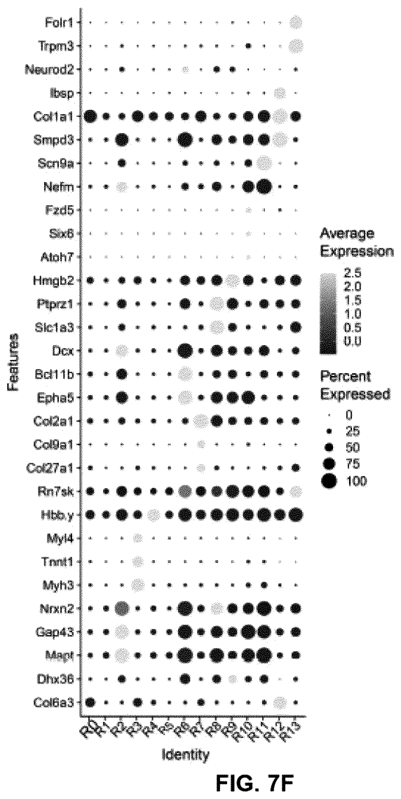

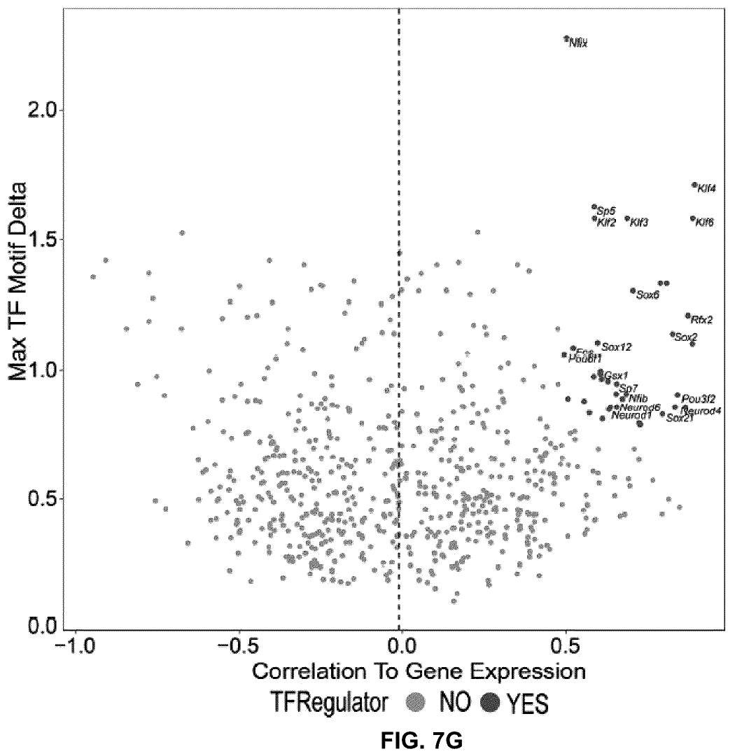

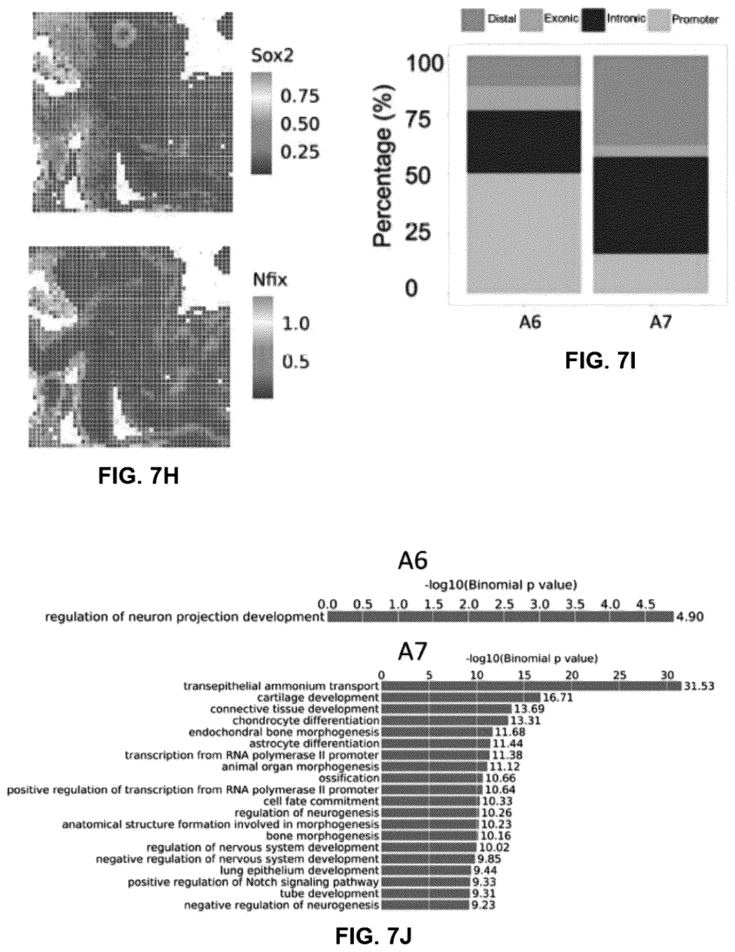

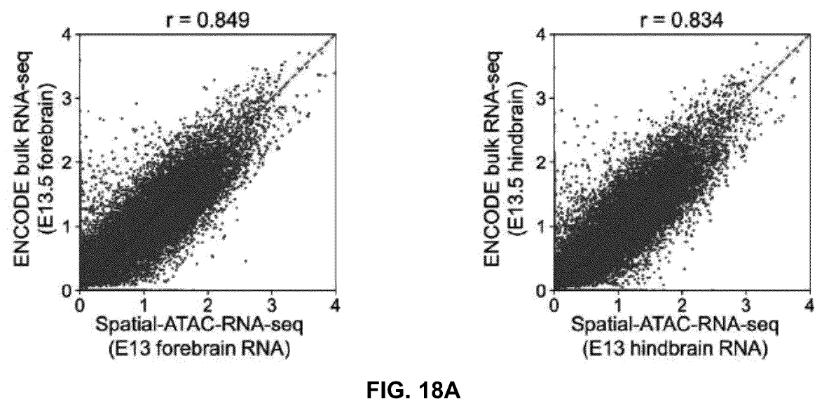

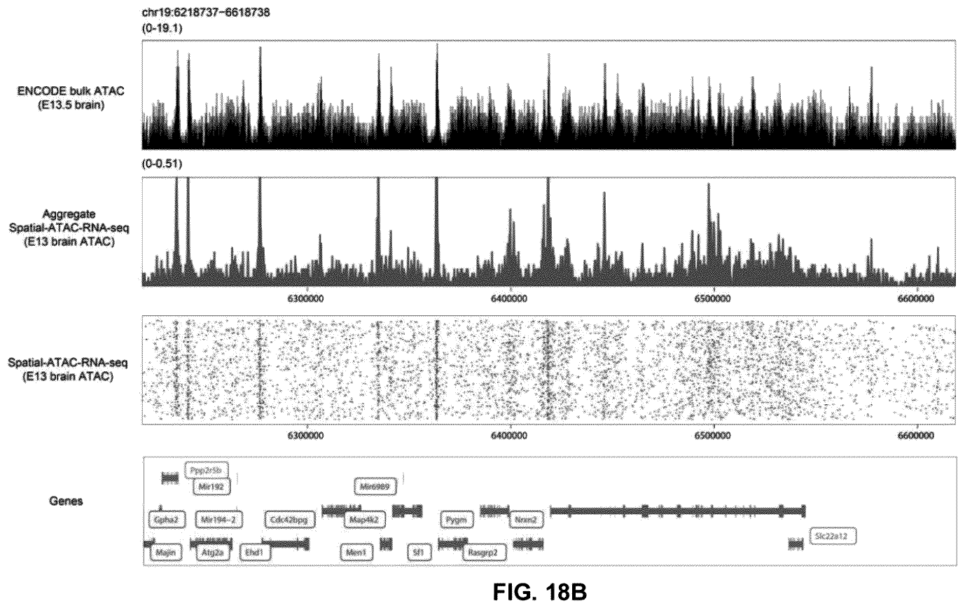

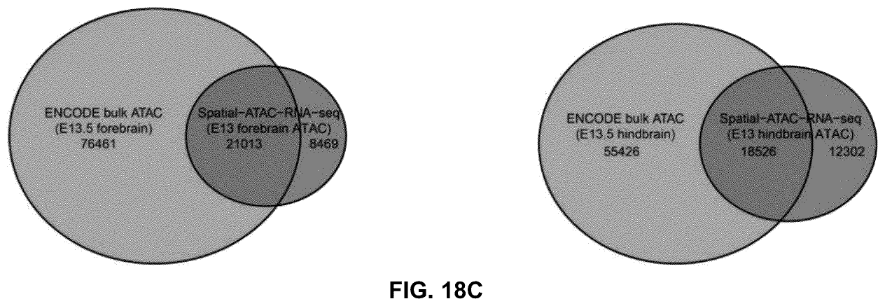

Spatial-ATAC-RNA-seq on E13 mouse embryo identified 8 major ATAC clusters and 14 RNA clusters, suggesting that at this stage of development, chromatin accessibility may not allow discrimination of all cell types defined by RNA expression. Spatial distribution of these clusters agrees well with tissue histology (see H&E staining of an adjacent tissue section, FIG. 1D and FIG. 7A). In the ATAC data, cluster A3 represents the embryonic eye field with open chromatin accessibility at the loci of genes like Six6 (FIG. 1D, left and FIG. 1E). Clusters A4 to A5 are associated with several developing internal organs. Clusters A6 to A7 cover the central nervous system (CNS). To benchmark the ATAC result, the chromatin accessibility profiles in the pixels within specific organs were aggregated and compared with organ-specific ENCODE E13.5 ATAC-seq reference data (FIG. 18B). Additionally, the peaks obtained from spatial ATAC data are consistent with the ENCODE reference (FIG. 18C). Spatial ATAC data was integrated with scRNA-seq (Cao, 2019, Nature, 566:496-502) data for cell type assignment in each cluster (FIG. 7C-FIG. 7D). As expected, radial glia (neural stem/progenitor cells) were observed predominantly in the ventricular layer, and the differentiated cell types such as postmitotic premature neurons and inhibitory interneurons were enriched in the regions distant from the ventricular layer (FIG. 7D).

Cell type-specific marker genes were then identified for individual clusters and their expression was inferred from chromatin accessibility (FIG. 1E, FIG. 7B, and FIG. 7E) and predicted by gene activity score (GAS (Granja, 2021, Nat Genet, 53:403-411), Methods). Sox2, which is involved in the development of nervous tissue and optic nerve formation (Gomez-Lopez, 2011, Glia, 59:1588-1599; Mihelec, 2009, European Journal of Human Genetics, 17:1417-1422), showed high chromatin accessibility in the embryonic eye field and in the ventricular layer containing neural stem/progenitor cells. Pax6 exhibited a similar spatial pattern of chromatin accessibility. Myt11, which encodes myelin transcription factor 1 like protein, presented higher ATAC signal in the embryonic brain and neural tube (Chen, 2021, Neuron, 109:3775-3792). Six6, a key gene involved in eye development (Diacou, 2018, Cell Reports, 25:2510-2523), showed highest GAS in the eye region. Nrxn2 accessibility which encodes Neurexin 2, a key gene in the vertebrate nervous system (Stelzer, 2016, Current Protocols in Bioinformatics, 54: 1.30.31-31.30.33), was observed extensively in most neural cells. Accessibility at Rbfox3, a splicing factor well known as the nuclear biomarker NeuN (Huang, 2022, Proceedings of the National Academy of Sciences, 119: e2203632119), was observed in neurons (Huang, 2022, Proceedings of the National Academy of Sciences, 119: e2203632119) while Sox1 and Sox2 presented enriched chromatin accessibility in the ventricular layer (FIG. 1E and FIG. 7B). Cell type-specific enrichment of transcription factor (TF) regulators were also examined using Chrom VAR (Schep, 2017, Nature Methods, 14:975-978) analysis of deviation of TF motifs (Sox2 and Nfix) and identified the positive TF regulators (FIG. 7G-FIG. 7H). It was observed that Sox2 motif was enriched in cluster A7, consistent with its function in embryonic brain development (Amador-Arjona, 2015, Proceedings of the National Academy of Sciences, 112: E1936-E1945). The GREAT analysis (McLean, 2010, Nature Biotechnology, 28:495-501) further verified the strong concordance between gene regulatory pathways and anatomical annotation (FIG. 7I-FIG. 7J).

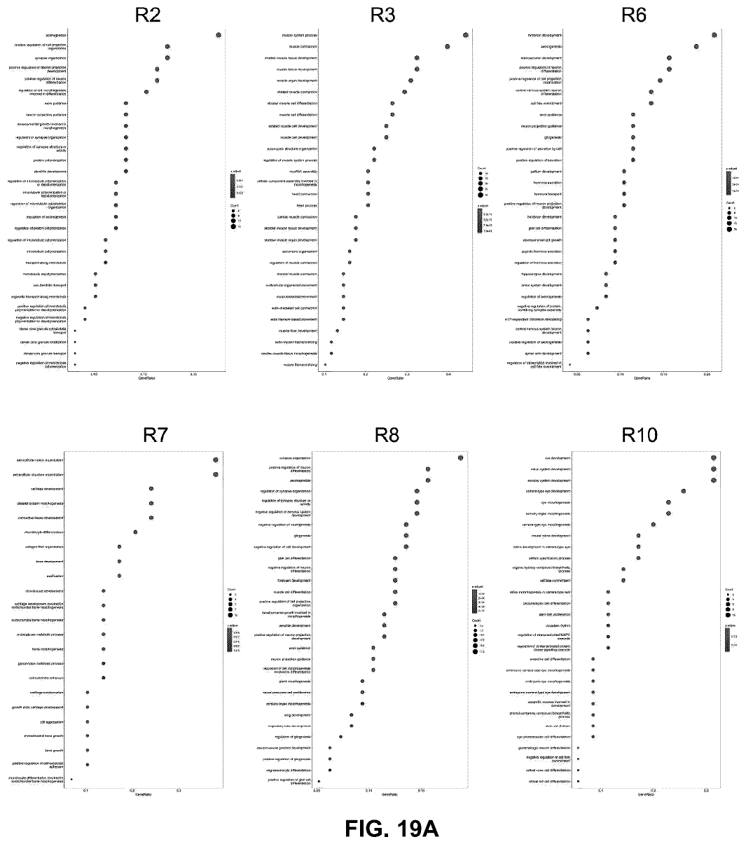

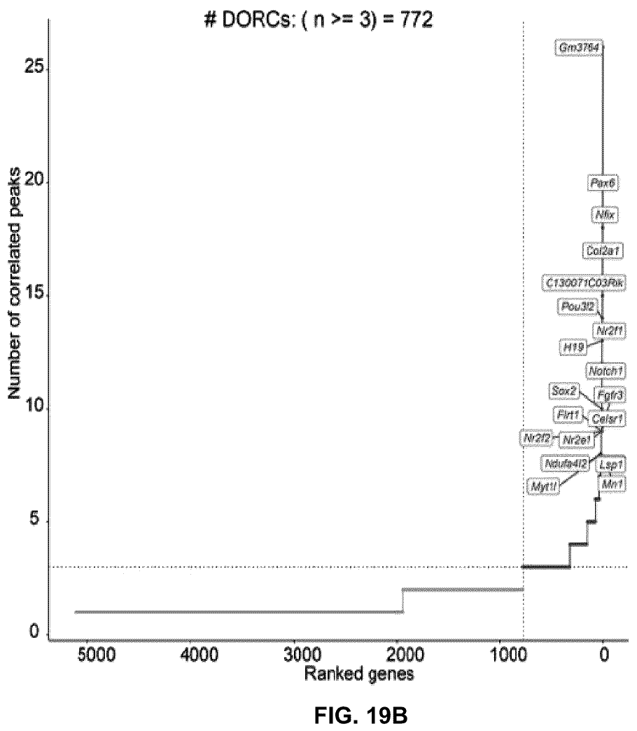

For the RNA spatial data, 14 distinct clusters were identified and characterized by specific marker genes (FIG. 1D, middle and FIG. 7F). For example, cluster R10 (Six6) was correlated with the embryonic eye. Clusters R2, R6, and R8 were related to the CNS. Cluster R7 was associated with the formation of cartilage. To evaluate the data quality, a correlation analysis was performed with organ-specific ENCODE E13.5 RNA-seq reference data, which showed high concordance (FIG. 18A). Cell type-specific marker genes were also identified for individual RNA clusters. For example, Mapt (R2) may function in establishing and maintaining neuronal polarity (Yoshida, 2012, Journal of Neurochemistry, 120:165-176). Epha5 (R6) is involved in axon guidance, while Slcla3 (R8) is involved in regulating excitatory neurotransmission in the CNS18. Myh3 (R3) is associated with muscle contraction (Zhao, 2022, NPJ Genomic Medicine, 7:11). Col2a1 (R7), which shows specific expression in cartilaginous tissues, plays an essential role in normal embryonic skeleton development (Stelzer, 2016, Current Protocols in Bioinformatics, 54:1.30.31-31.30.33). Pathway analysis (Wu, 2021, Innovation, 2:100141) results agree with anatomical annotation (FIG. 19A).

Integration of the spatial RNA data with scRNA-seq mouse organogenesis data (Cao, 2019, Nature, 566:496-502) was performed to identify the cell identities in each pixel (FIG. 7C-FIG. 7D). It was observed that the radial glia, postmitotic premature neurons, and inhibitory interneurons (clusters R8, R6, R2) were present in the same major clusters as shown in the ATAC analysis (clusters A7, A6), which verified the use of multiple omics information for robust cell type identification. Joint clustering of spatial ATAC and RNA data to refine the spatial patterns was attempted. A new neuronal cluster (J10) was identified in the joint clustering analysis, which was not readily resolved by single modalities alone (FIG. 1D, right). This result highlights the unique value to use joint multi-omics profiles for improving the cell-type-specific spatial mapping (Zhu, 2021, Nature Methods, 18:283-292).

Co-profiling of epigenome and transcriptome allows for investigating the correlation between accessible peaks and expressed genes pixel by pixel in the tissue context. Distinct signals at predicted enhancers for some genes (Sox2, Pax6, and Sox1) was observed (FIG. 7E). For example, enhancers for Sox2 and Pax6, had higher chromatin accessibility in cluster A7 and A3, respectively, suggesting their roles in these tissue regions to regulate Sox2 and Pax6 expression. Interestingly, although spatial RNA distribution of these genes corresponded with their chromatin accessibility, the expression level may differ significantly from the degree of accessibility (FIG. 1E and FIG. 7B). Some marker genes (i.e., Pax6, Sox2, and Myt11) were highly accessible in some regions of the embryonic brain but showed modest or low RNA expression (FIG. 1E and FIG. 7B), which may indicate the lineage priming (Meijer, 2022, Neuron, 110:1193-1210) of these genes in embryonic brain development (FIG. 19B) (Ma, 2020, Cell, 183:1103-1116). Despite a strong correlation between replicates (FIG. 17D), this observation could be due in part to the sequencing depth and the RNA detection sensitivity. Nevertheless, these results highlight the potential to link genome-wide epigenetic regulation to transcription in the spatial tissue context.

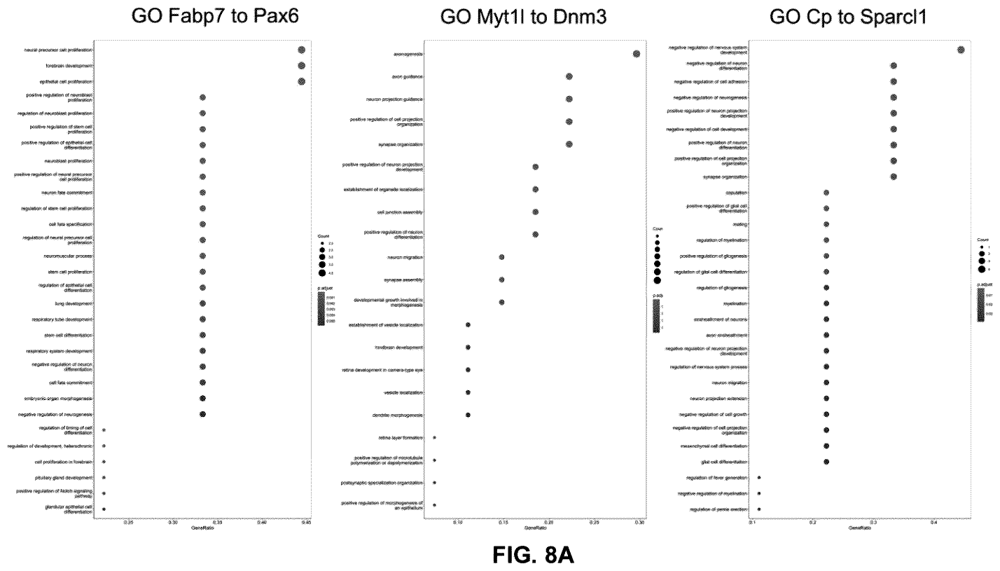

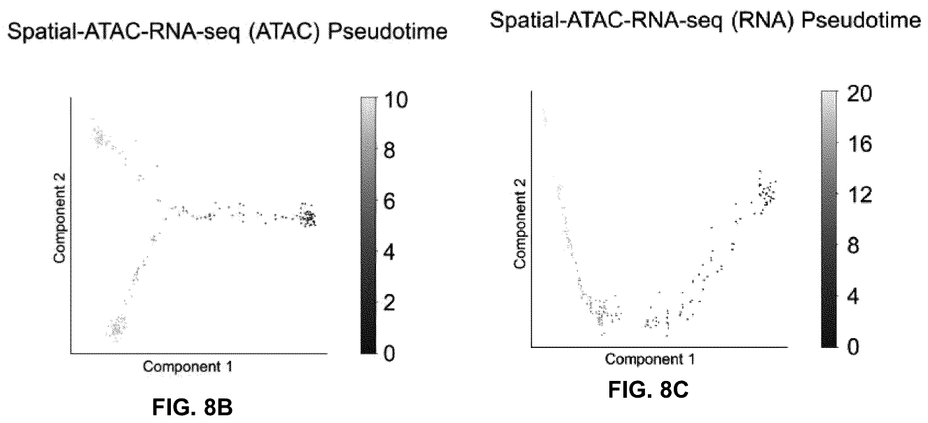

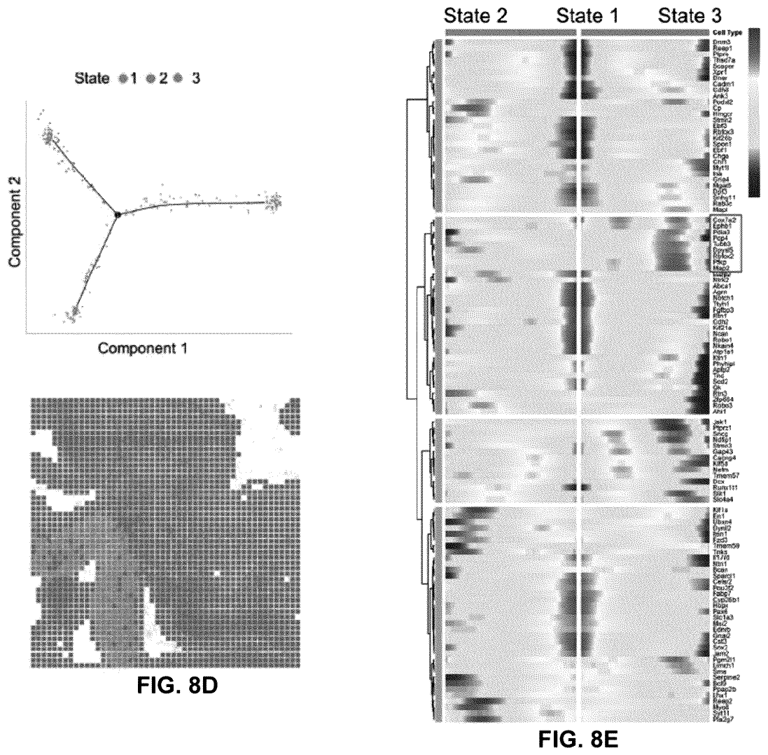

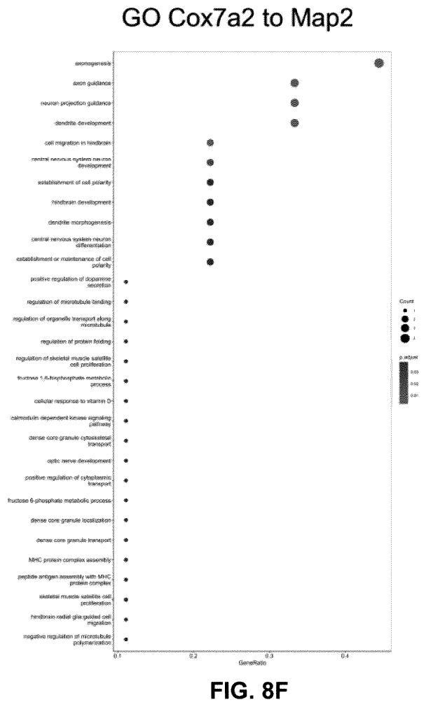

To investigate the spatiotemporal relationship between chromatin accessibility and gene expression in embryonic development, the differentiation trajectory from radial glia to various types of neurons such as postmitotic premature neurons (Kriegstein, 2009, Annual Review of Neuroscience, 32:149-184), was analyzed. The pseudotime analysis (Trapnell, 2014, Nat Biotechnol, 32:381-386) was conducted under the ATAC pseudotime coordinate system, and the developmental trajectories were directly visualized in the spatial tissue map (FIG. 1F). Chromatin accessibility GAS and gene expression along this trajectory revealed dynamic changes in selected marker genes (FIG. 1G-FIG. 1H). As expected, the expression levels of Sox2, Pax6 and other genes involved in progenitor maintenance and proliferation (Gene Ontology (GO) Fabp7 to Pax6 in FIG. 8A) were downregulated during the transition to postmitotic neurons. Interestingly, the loss of chromatin accessibility at the Pax6 and the radial glia marker Fabp7 loci preceded the downregulation of the corresponding RNAs (FIG. 1H). In turn, genes involved in neuronal identity, axonogenesis and synapse organization (GO Myt11 to Dnm3 in FIG. 8A) such as Dcx and Tubb3 exhibited an increased expression in the spatial pseudotime, but the chromatin accessibility at their loci was already elevated at earlier stages, suggesting lineage-priming of these genes in expression (Ma, 2020, Cell, 183:1103-1116; Meijer, 2022, Neuron, 110:1193-1210). A cohort of genes whose expression quickly declined during the spatial pseudotime, but whose chromatin accessibility was maintained throughout the pseudotime or only declined at very late stages was also found. Many of these genes such as Ptprz1, Bcan, Luzp2 are characteristic of oligodendrocyte precursor cells and the corresponding GO biological processes were negative regulation of myelination and regulation of gliogenesis (GO Cp to Sparcl1 in FIG. 8A), suggesting that the neuronal lineage might retain the potential to acquire an oligodendrocyte identity even when they have already migrated away from the ventricular zone in the embryonic brain. Interestingly, Monocle2 pseudotime analysis revealed a bifurcation in chromatin accessibility but not in RNA expression; one path led to regions close to the ventricular zone (green pixels, FIG. 8D-FIG. 8E) and the other terminated in regions distal from the ventricular zone (blue pixels). In contrast to the green path, the blue path presented an increase in chromatin accessibility for genes involved in axonogenesis and dendrite formation (FIG. 8E, red box and FIG. 8F), suggesting that the chromatin state of neural cells distal to the ventricle corresponds to a more differentiated neuronal state. Thus, spatial-ATAC-RNA-seq as described herein can be used to decipher the gene regulation mechanism and spatiotemporal dynamics during tissue development.

### Spatial-ATAC-RNA-Seq of Mouse Brain

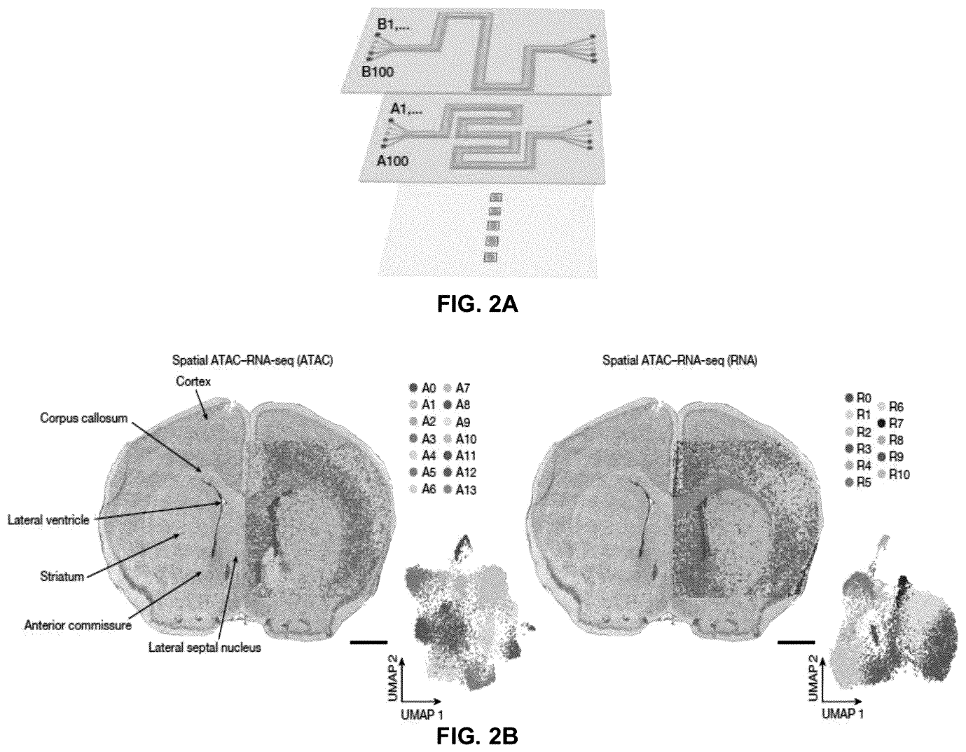

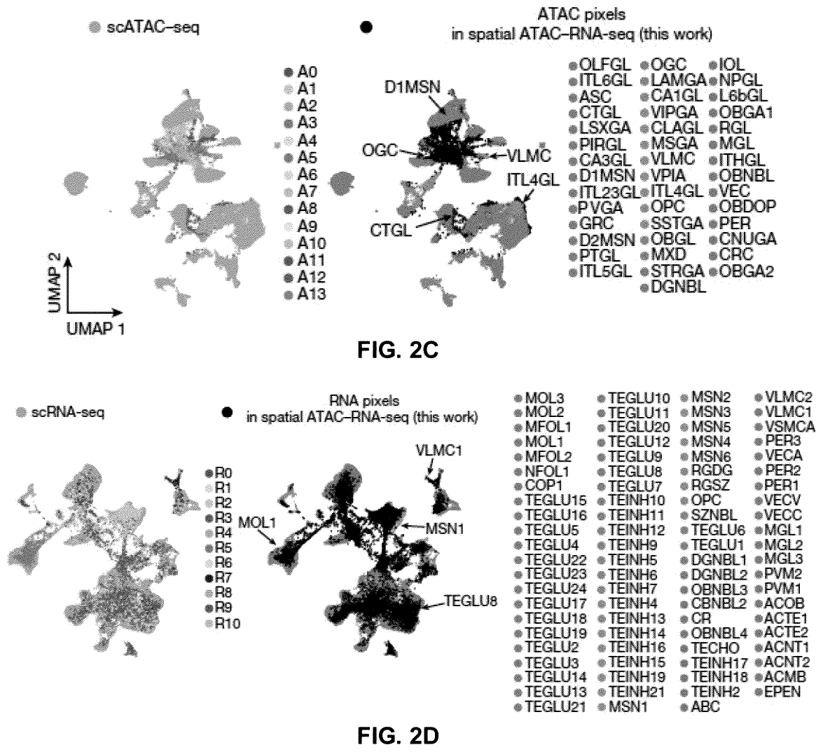

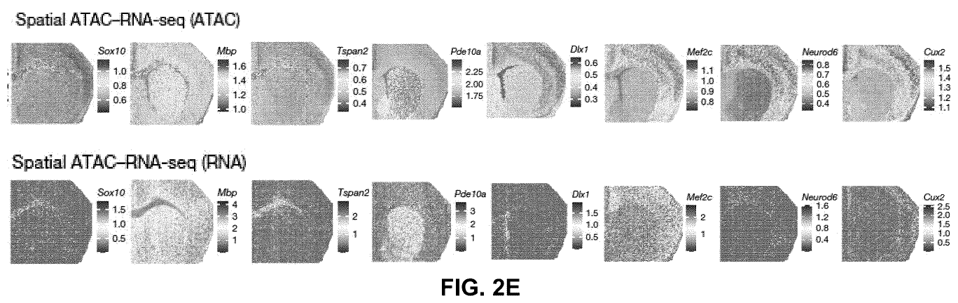

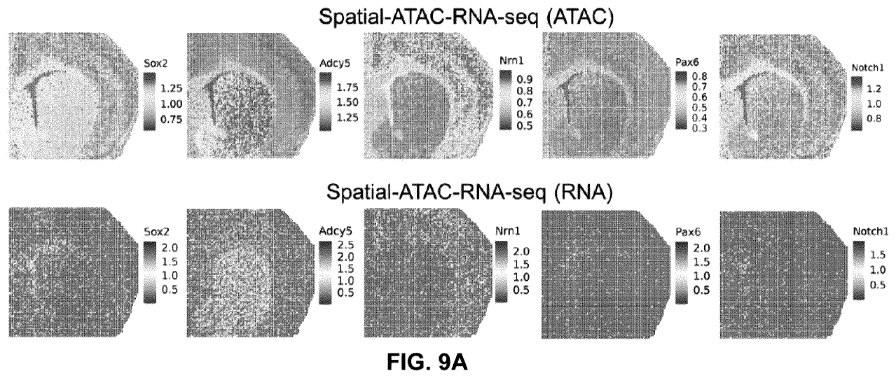

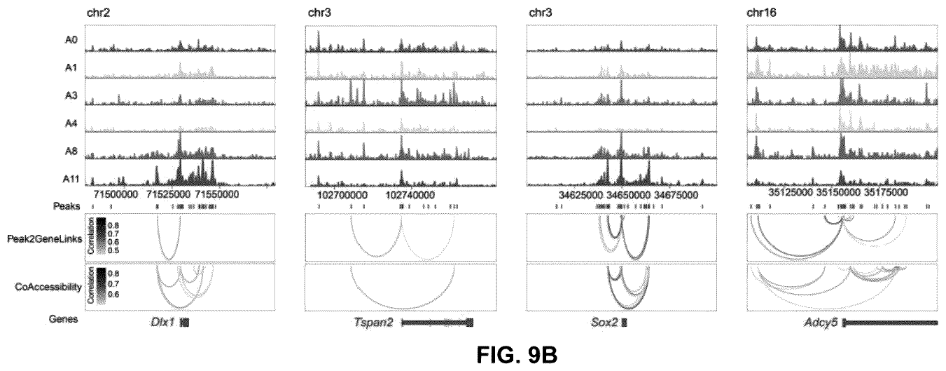

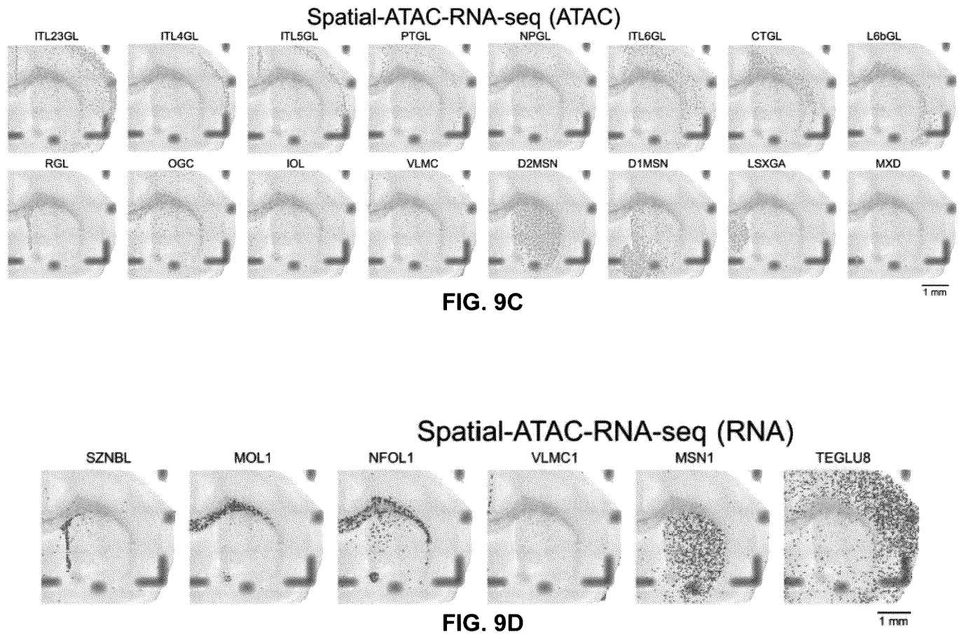

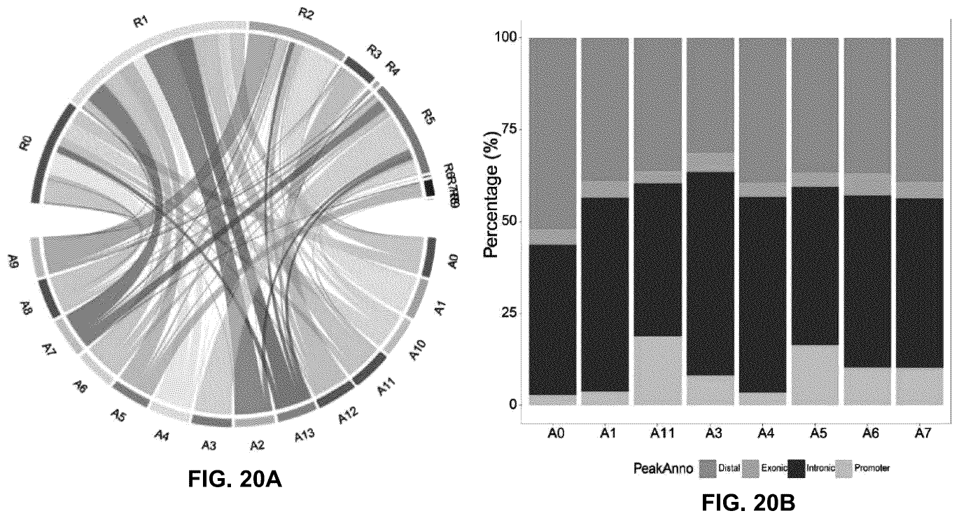

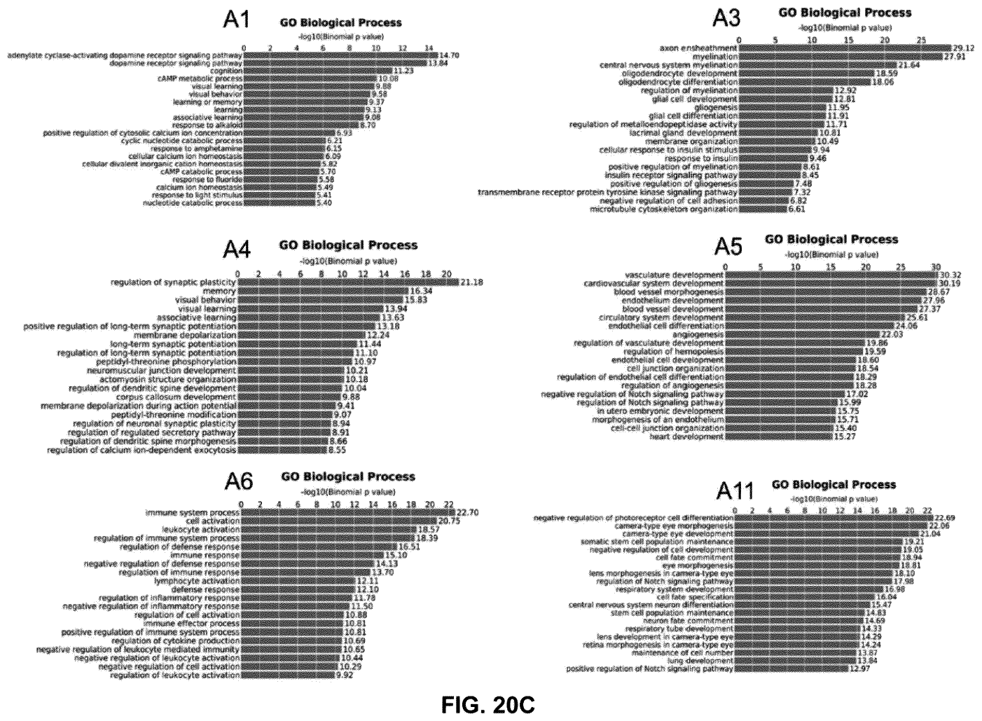

A new microchip with 100 serpentine microchannels to barcode 100×100 or totally 10,000 pixels per tissue section and up to 5 samples per run (FIG. 2A, 20 μm pixel size) was developed. It was applied to P22 mouse brain coronal sections (at Bregma 1) for profiling chromatin accessibility jointly with transcriptome. In contrast to E13 mouse embryonic brain, it showed a larger number of ATAC clusters (fourteen) compared to the RNA clusters (eleven), indicating terminal differentiation of most major cell types in the juvenile brain in contrast to that in embryo which consists of many undifferentiated or multipotent cell states. Spatial distribution of clusters agreed with anatomical annotation defined by the Nissl staining, reflecting arealization of the juvenile brain (FIG. 2B). Moreover, spatial clusters between ATAC and RNA showed strong concordance in cluster assignment (FIG. 20A). Chromatin accessibility of marker genes identified the major regions such as striatum (Pde10a, Adcy5, markers of medium spiny neurons, cluster A1), corpus callosum (Sox10, Mbp, and Tspan2, markers of oligodendroglia, cluster A3), cortex (Mef2c, Neurod6, and Nrn1, cluster A0 and A4, markers of excitatory neurons; Cux2, cluster A4), and lateral ventricle (Dlx1, Pax6, Notch1, and Sox2, markers of ependymal/neural progenitor cells, cluster A11) (FIG. 2E and FIG. 9A-FIG. 9B). The GREAT analysis confirmed that the major pathways correlated with the tissue functions in different anatomical regions (FIG. 20B-FIG. 20C). Spatial RNA clusters also showed region-specific gene expression such as striatum (Pde10a, cluster R2, medium spiny neurons), corpus callosum (Mbp, Tspan2, cluster R5, oligodendroglia), and cortex (Mef2c, cluster R0 and R1, excitatory neurons) (FIG. 2E and FIG. 9A).

Integration of single-cell ATAC-seq mouse brain atlas data (Li, 2021, Nature, 598:129-136) with spatial-ATAC-seq data allowed for identifying all major cell types, then label transfer was used to assign cell types to spatial locations where the epigenetic state may control specific cell type formation (FIG. 2A and FIG. 9C). For example, immature oligodendrocytes (IOL) and oligodendrocytes (OGC) in cluster A3 corresponding to corpus callosum were observed. A thin layer of radial glia-like cells (RGL) was found in the lateral ventricle, medium spiny neurons (D1MSN and D2MSN) in striatum, and inhibitory neurons in lateral septal nucleus (LSXGA, FIG. 9C) (Li, 2021, Nature, 598:129-136) based upon chromatin accessibility. Furthermore, the subclasses of excitatory neurons in different layers of cortex (ITL23GL, Cortex L2/3; ITL4GL, Cortex L4; ITL5GL, Cortex L5; PTGL, Cortex PT; NPGL, Cortex NP; ITL6GL, Cortex L6; CTGL, Cortex CT; and L6bGL, Cortex L6b) were clearly identified (Tasic, 2018, Nature, 563:72-78) (FIG. 9C). Integration of spatial RNA data with juvenile CNS scRNA-seq atlas (Zeisel, 2018, Cell, 174:999-1014) allowed for visualizing dominant transcriptional cell types in tissue (FIG. 2D and FIG. 9D). A high degree of concordance in spatial cell type distribution determined by ATAC vs RNA was observed, suggesting a general congruence between chromatin accessibility and transcriptome to define cell identities in tissue. For instance, the neuronal intermediate progenitor cells (SZNBL, included in RGL from scATAC-seq (Li, 2021, Nature, 598:129-136)), mature oligodendrocytes (MOL, included in OGC from scATAC-seq), newly formed oligodendrocytes (NFOL, included in IOL from scATAC-seq), and medium spiny neuron (MSN) revealed by the RNA data were enriched in the same regions as identified by the ATAC data (FIG. 9C-FIG. 9D). Furthermore, the detection of a thin layer of SZNBL cells in the lateral ventricle and vascular leptomeningeal cell (VLMC) cells demonstrated a high spatial resolution for this technology to identify low abundance cells and even at near-single cell resolution (for VLMC) (FIG. 9C-FIG. 9D).

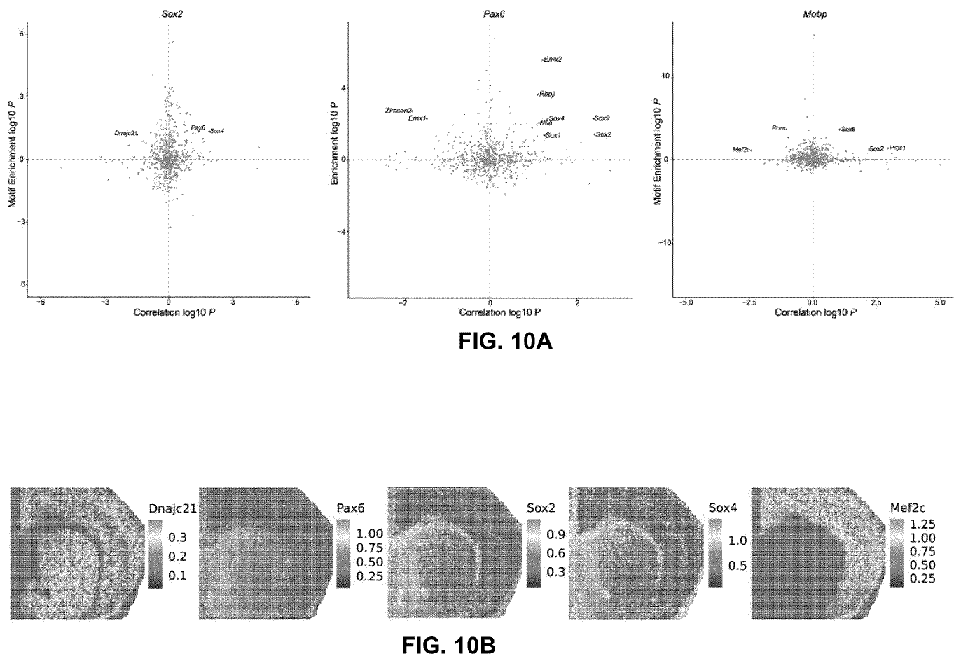

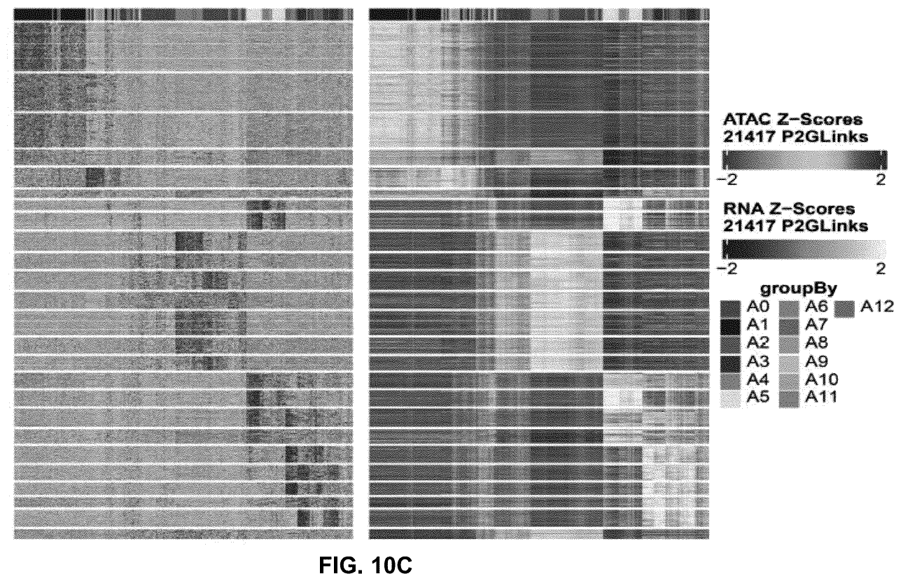

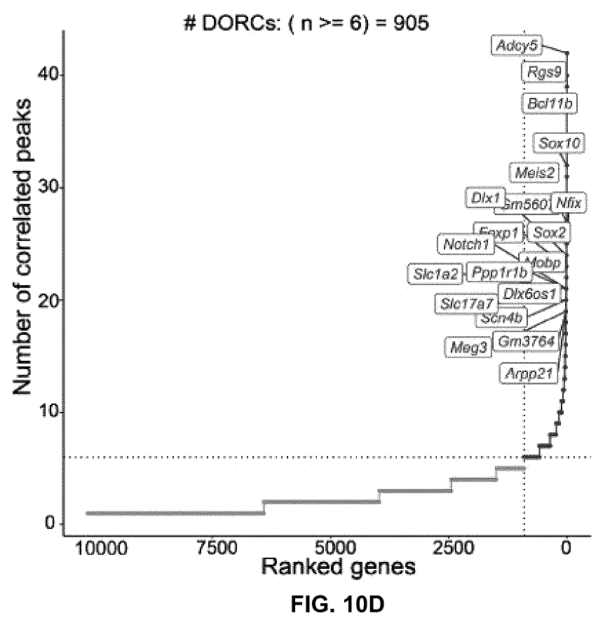

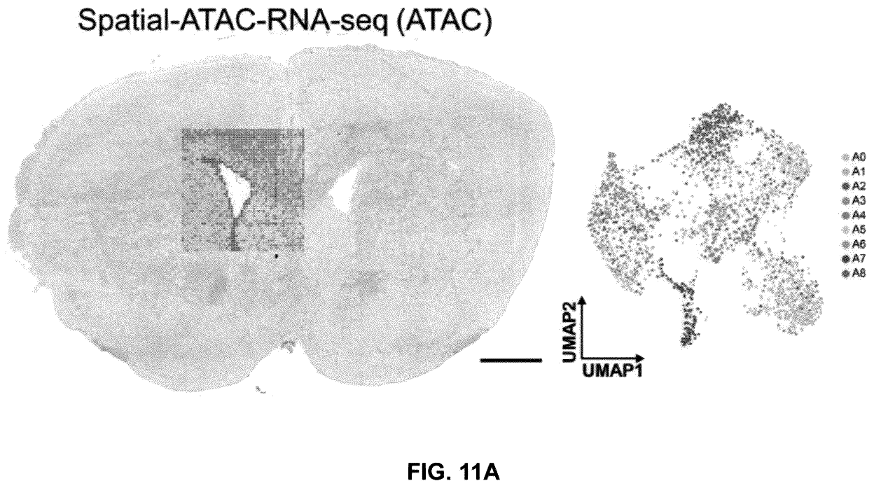

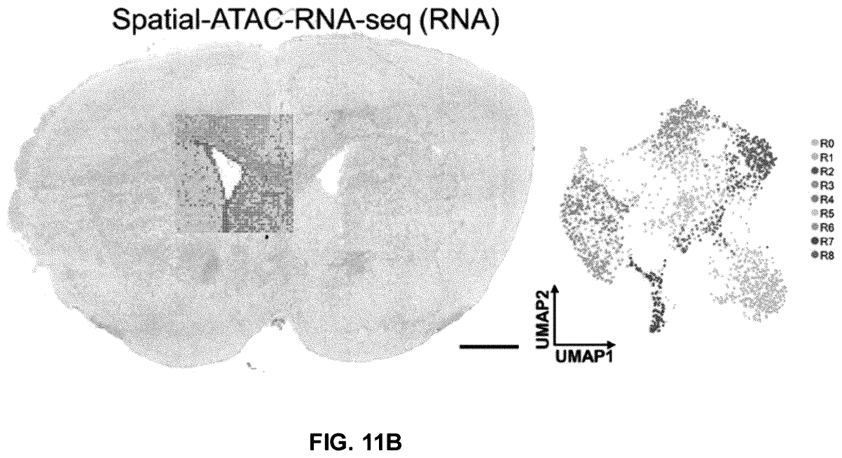

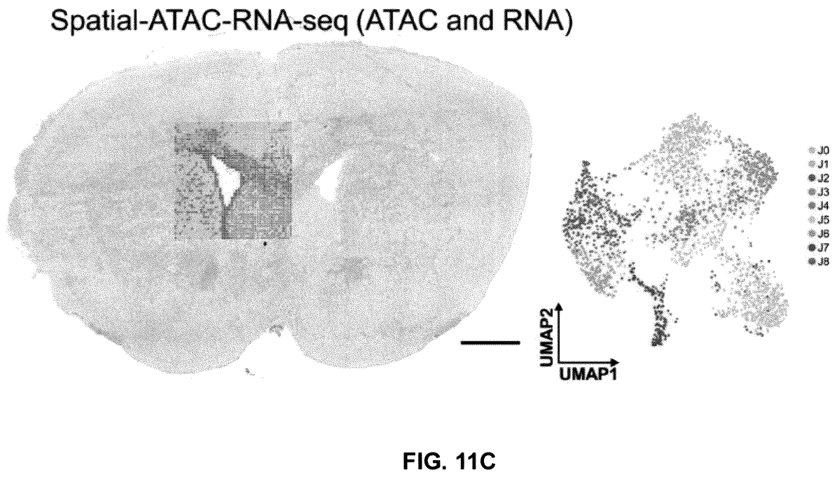

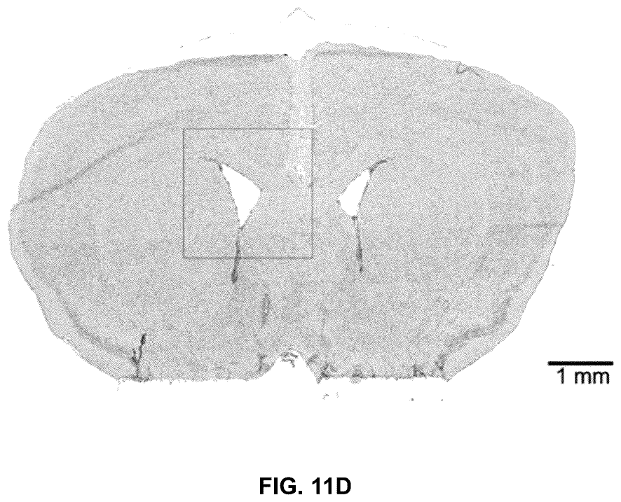

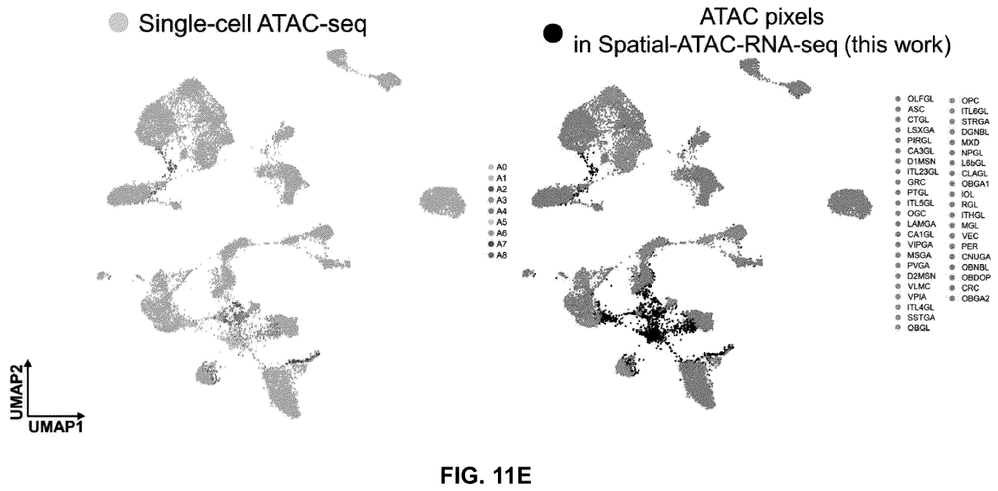

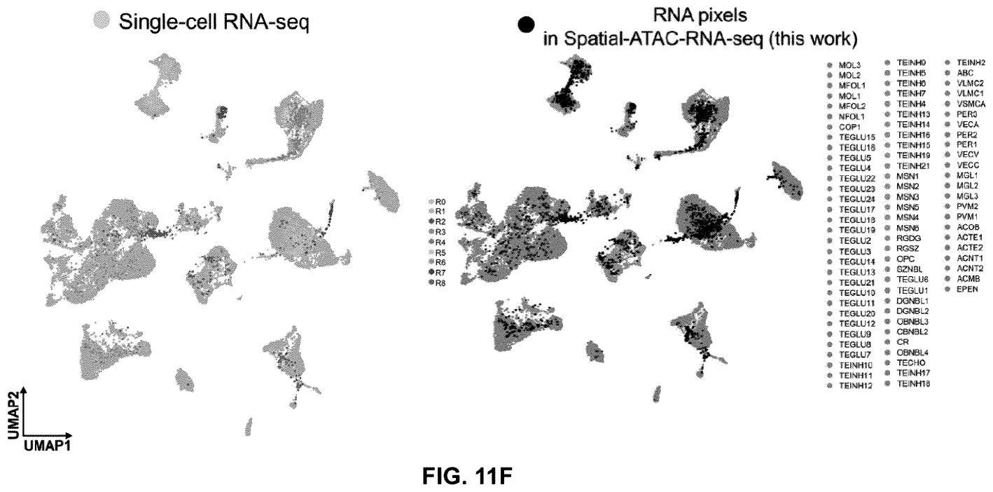

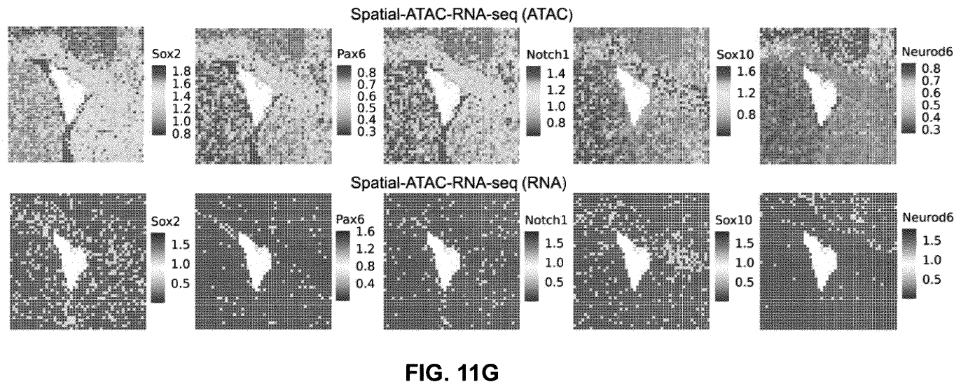

Joint profiling of ATAC and RNA allows for inferring gene regulatory landscape by searching the correlated peak accessibility and gene expression. 21,417 significant peak-to-gene linkages between regulatory elements and target genes were detected (FIG. 10C). Some potential enhancers with dynamically regulated promoter interactions were found to be enriched in specific clusters such as Dlx1 and Sox2 (cluster A11), Tspan2 (cluster A3), and Adcy5 (cluster A1) (FIG. 9B), indicating the ability for spatial-ATAC-RNA-seq to identify the key regulatory regions for target genes. Spatial patterns of TF motif enrichment (Dnajc21, Pax6, Sox2, Sox4, and Mef2c) provided further insights into the TF regulators33 visualized in tissue (FIG. 10A-FIG. 10B). For example, Sox2 was examined for chromatin accessibility, gene expression, putative enhancers, and TF motif enrichment simultaneously, enabling a more comprehensive understanding of gene regulation dynamics. In the embryonic mouse brain, some marker genes with open chromatin accessibility (i.e., Sox10, Sox2, Neurod6, Pax6, and Notch1) were not or lowly expressed in transcription (FIG. 2E and FIG. 9A). A biological replicate experiment on P21 mouse brain was also performed (FIG. 11, 20 μm pixel size, 50×50 barcodes). Many of these genes encode for transcription factors involved in neural development, suggesting the possibility of epigenetic but not transcriptional memory of these genes in brain development (FIG. 10D) (Ma, 2020, Cell, 183:1103-1116).

### Spatial-CUT&Tag-RNA-Seq of Mouse Brain

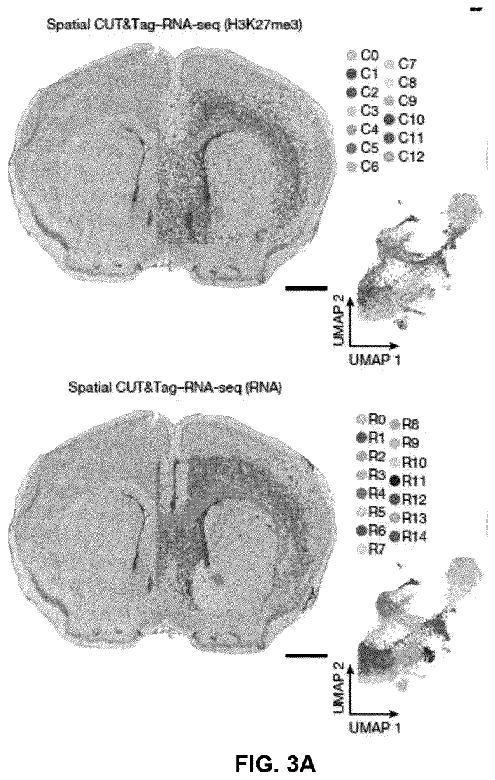

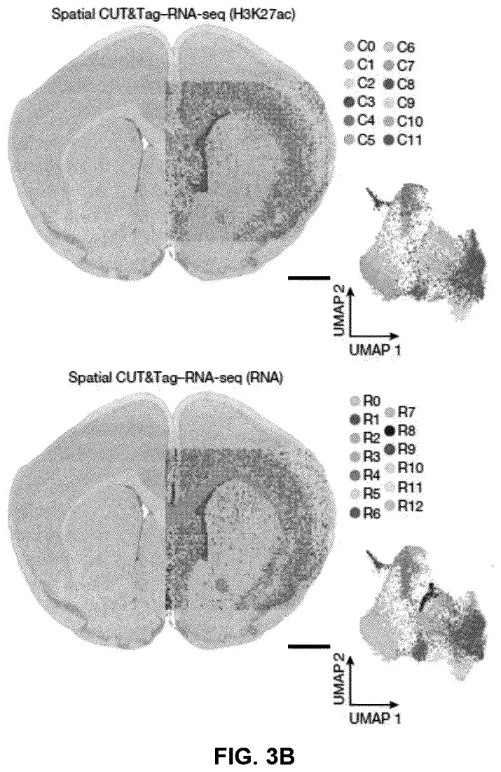

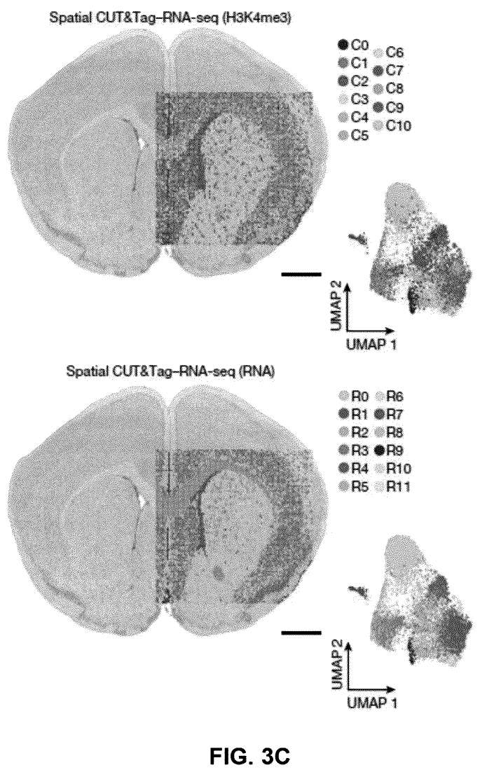

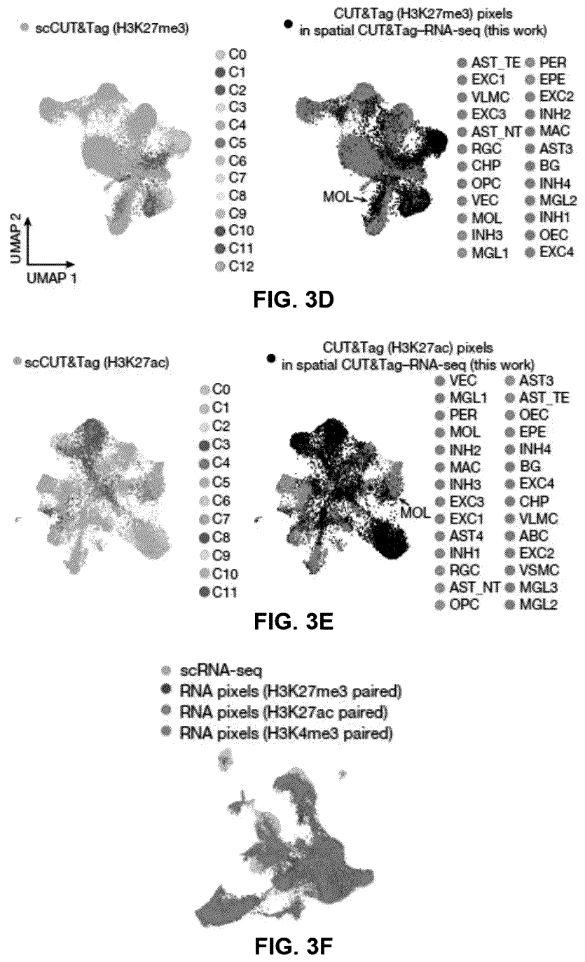

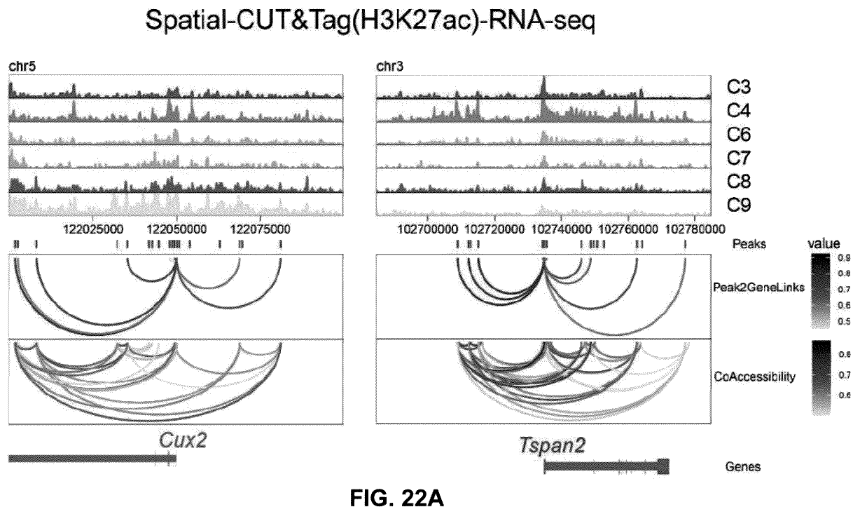

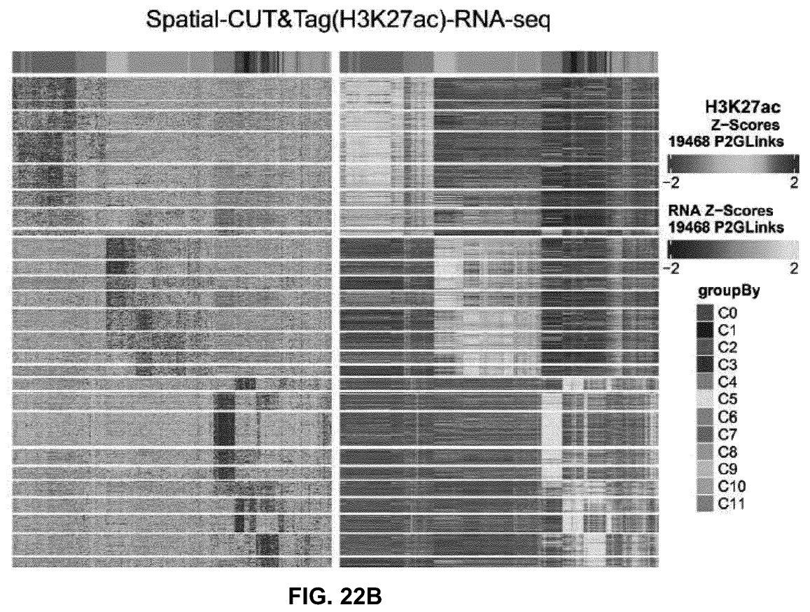

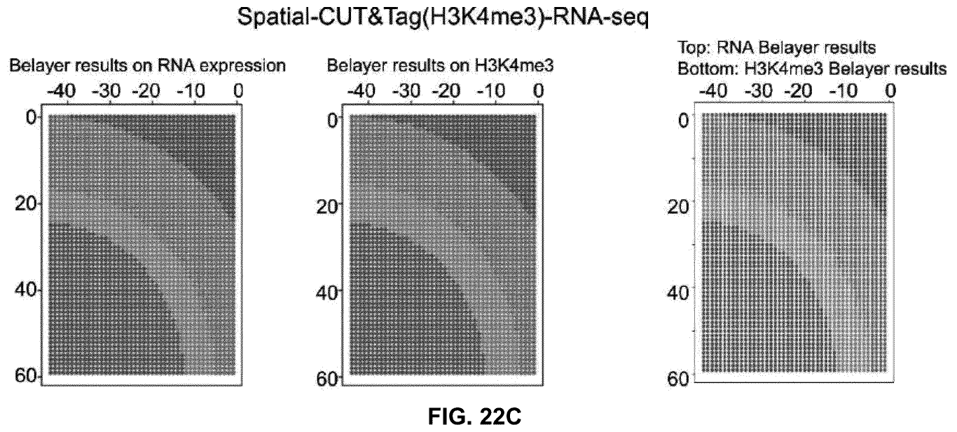

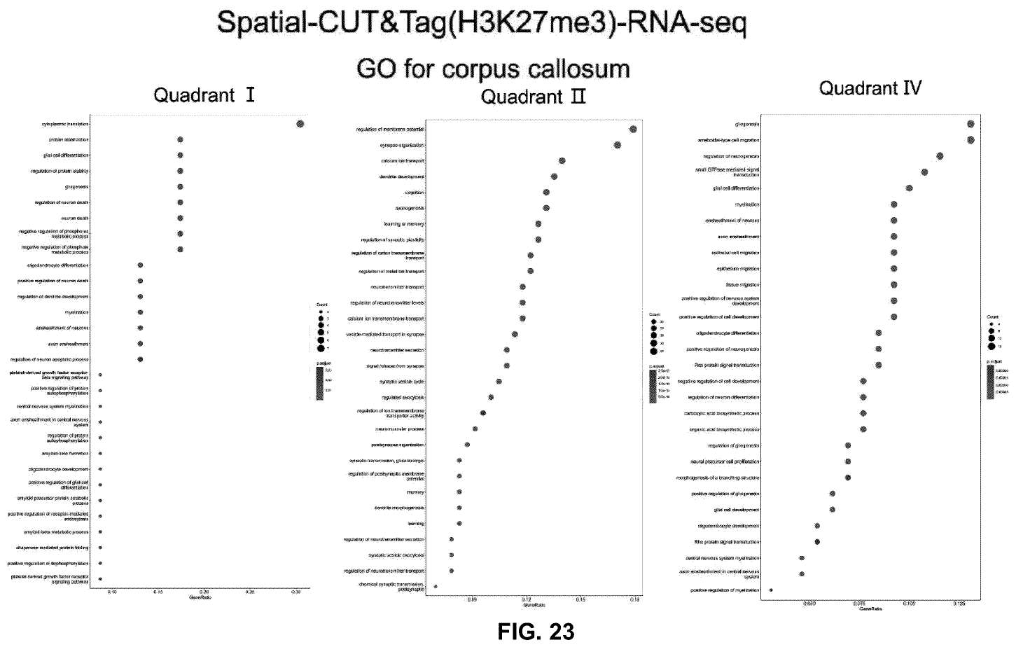

In addition to chromatin accessibility, histone modifications are also important aspects in epigenetic regulation. Spatial-CUT&Tag-RNA-seq was performed to co-profile transcriptome and H3K27me3 (repressing loci), H3K27ac (activating promoters and/or enhancers), or H3K4me3 (active promoters) histone modifications, respectively, in P22 mouse brain (20 μm pixel size, 100×100 barcodes). 13 and 15 specific clusters for H3K27me3 and RNA, 12 and 13 clusters for H3K27ac and RNA, 11 and 12 clusters for H3K4me3 and RNA, respectively were identified (FIG. 3A-FIG. 3C). These clusters agreed well with the anatomical annotation by the Nissl staining and showed good concordance between CUT&Tag and RNA in spatial patterns (FIG. 3A-FIG. 3C), which was further confirmed by the Belayer (Ma, 2022, Cell, 13:786-797) analysis (FIG. 22C).

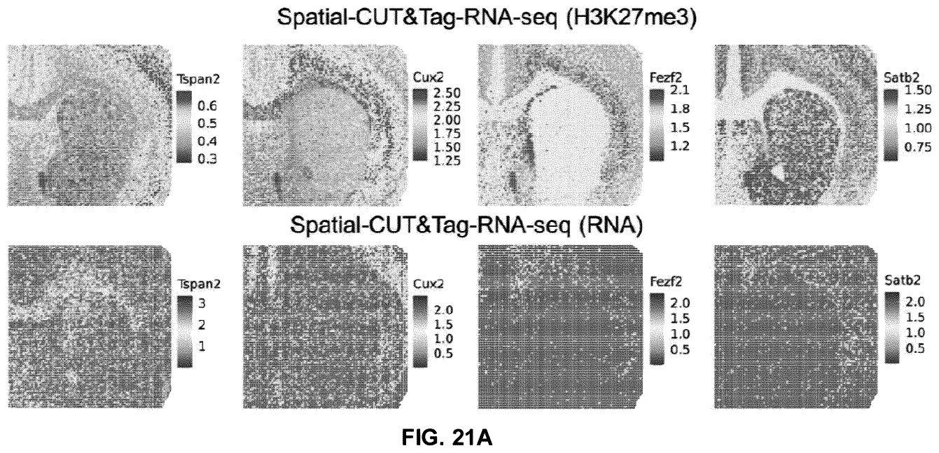

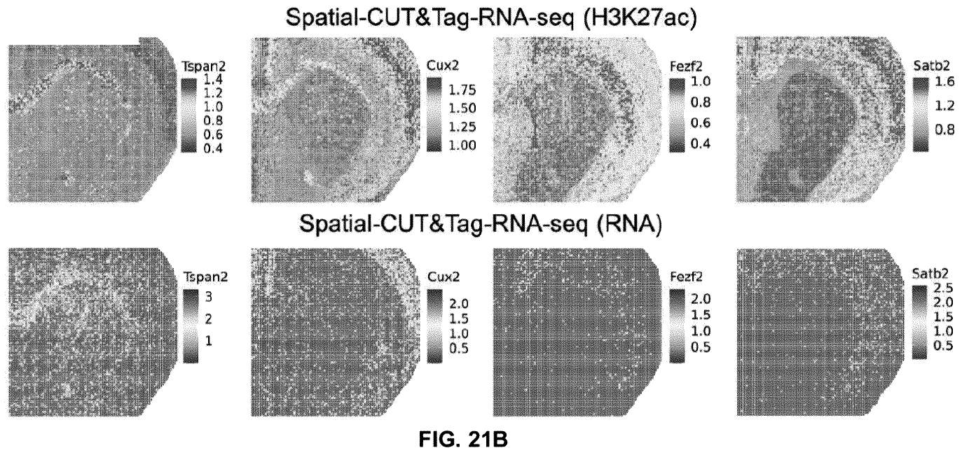

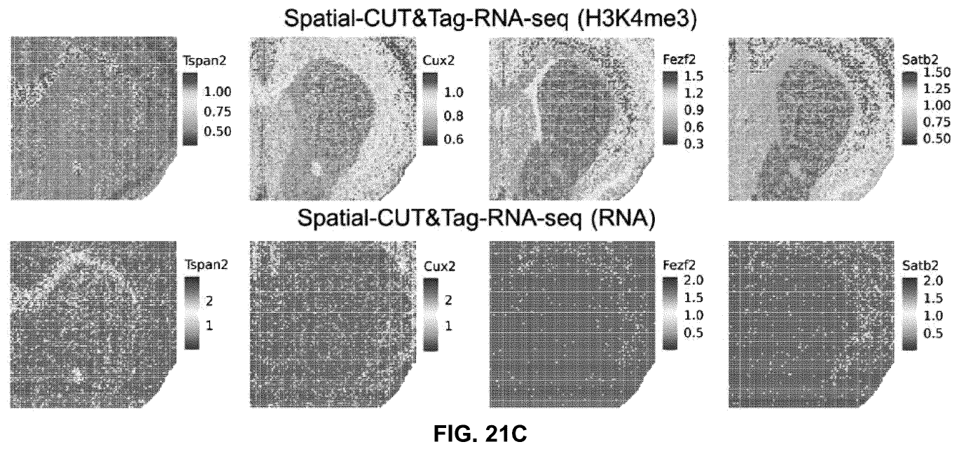

For H3K27me3, the gene expression was predicted by calculating chromatin silencing score (CSS (Deng, 2022, Science, 375:681-686; Wu, 2021, Nature Biotechnology, 39:819-824), Methods). High CSS indicates repressed genes because of the transcriptional repression function of H3K27me3. For H3K27ac and H3K4me3, the active genes should correspond to high gene activity score (GAS (Deng, 2022, Science, 375:681-686; Granja, 2021, Nat Genet, 53:403-411), Methods). Clustering of CSS or GAS based on different modifications resolved all major tissue regions (FIG. 3A-FIG. 3C and FIG. 21) and identified region-specific marker gene modifications. Cux2 showed high GAS in cluster C9 (H3K27ac) and cluster C6 (H3K4me3), indicating the enrichment of excitatory neurons5. Cux2 was observed in cortical layers 2/3 corresponding to superficial layers in the cortex. In contrast, H3K27me3 was depleted at Cux2 in the same region (cluster C3) (FIG. 21). GAS of Fezf2, a marker gene for cortical layer 5, was high in cluster C8 (H3K27ac) and cluster C7 (H3K4me3) corresponding to the deeper layers of the cortex. Fezf2 was depleted for H3K27me3 (cluster C1) in these layers. Satb2, showed the highest activity in clusters C8, C9 (H3K27ac) and clusters C6, C7 (H3K4me3) but the lowest CSS in clusters C1, C3 (H3K27me3) in the cortical layer. Tspan2 was enriched in cluster C4 (H3K27ac) or cluster C1 (H3K4me3), but depleted in cluster C5 (H3K27me3) in corpus callosum, associated with oligodendrocyte lineage development (FIG. 21). The corresponding RNA clusters also showed concordant region-specific signatures (FIG. 3A-FIG. 3C and FIG. 21) as exemplified by Cux2 in cortical layer 2/3 excitatory neurons (cluster R5 (H3K27me3 paired), cluster R5 (H3K27ac paired), and cluster R6 (H3K4me3 paired)), Fezf2 in cortical layer 5 excitatory neurons (cluster R1 (H3K27me3 paired), cluster R1 (H3K27ac paired), and cluster R1 (H3K4me3 paired)), and Tspan2 in oligodendrocyte lineage cells in corpus callosum (cluster R4 (H3K27me3 paired), cluster R4 (H3K27ac paired), and cluster R3 (H3K4me3 paired), FIG. 21).

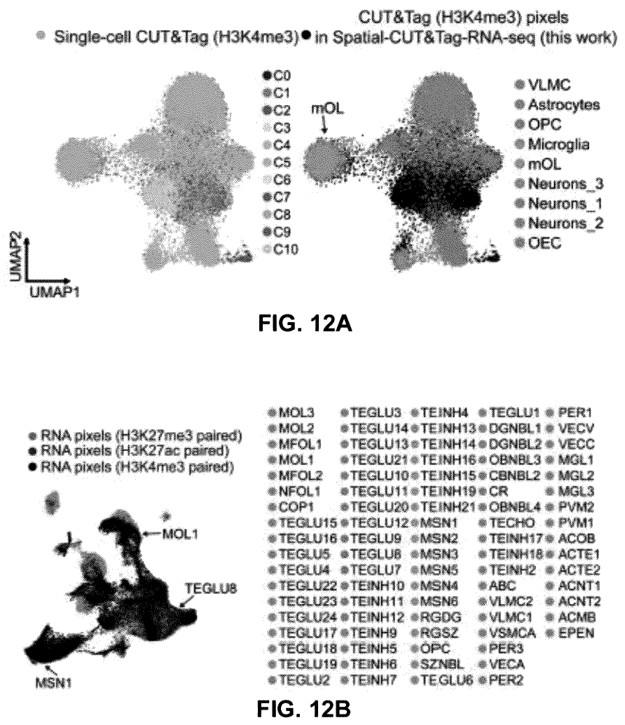

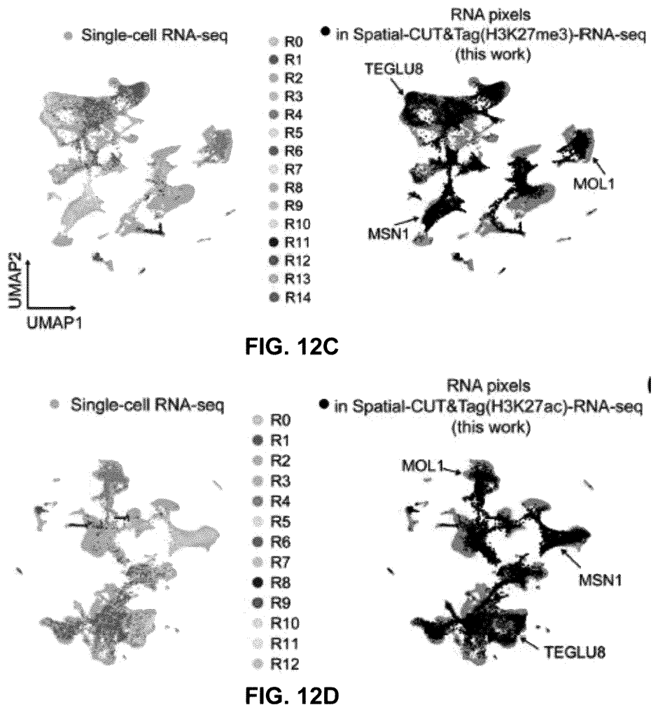

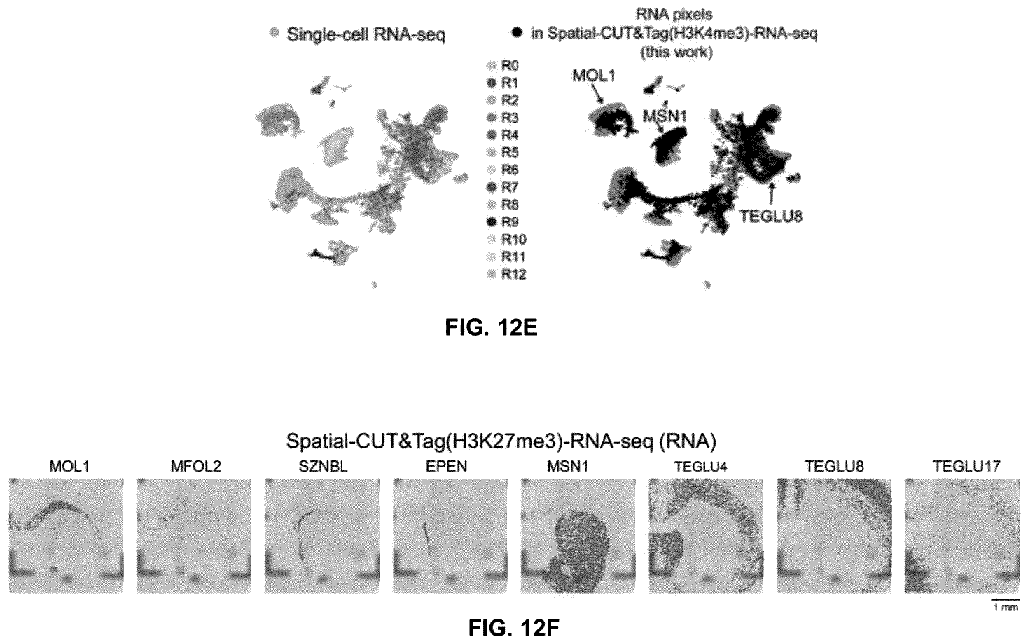

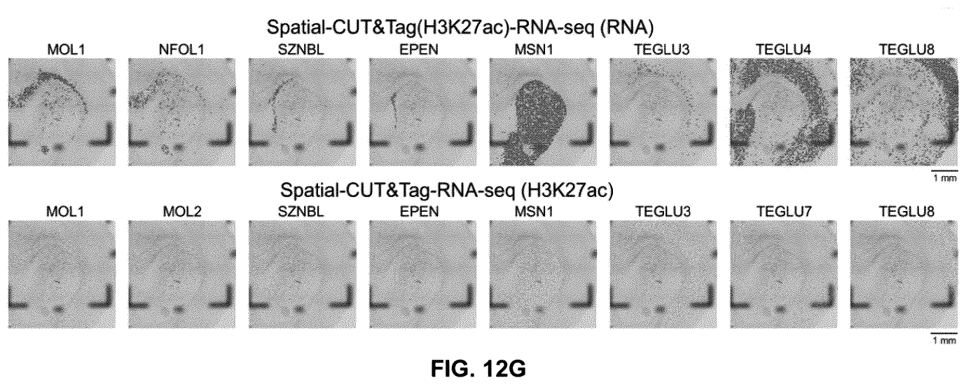

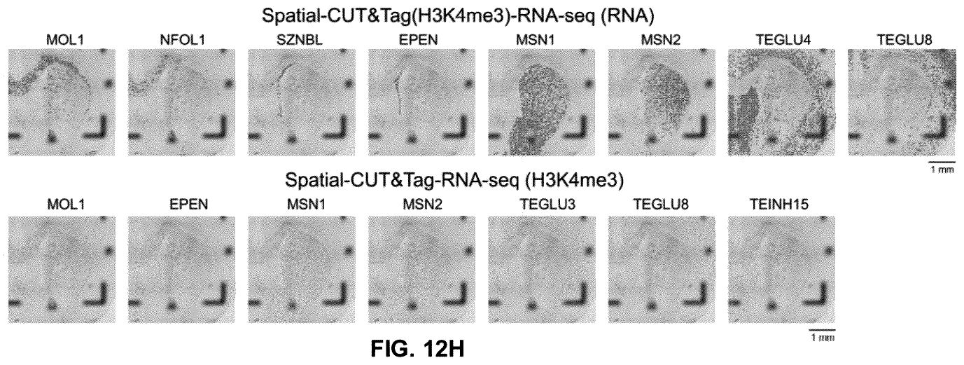

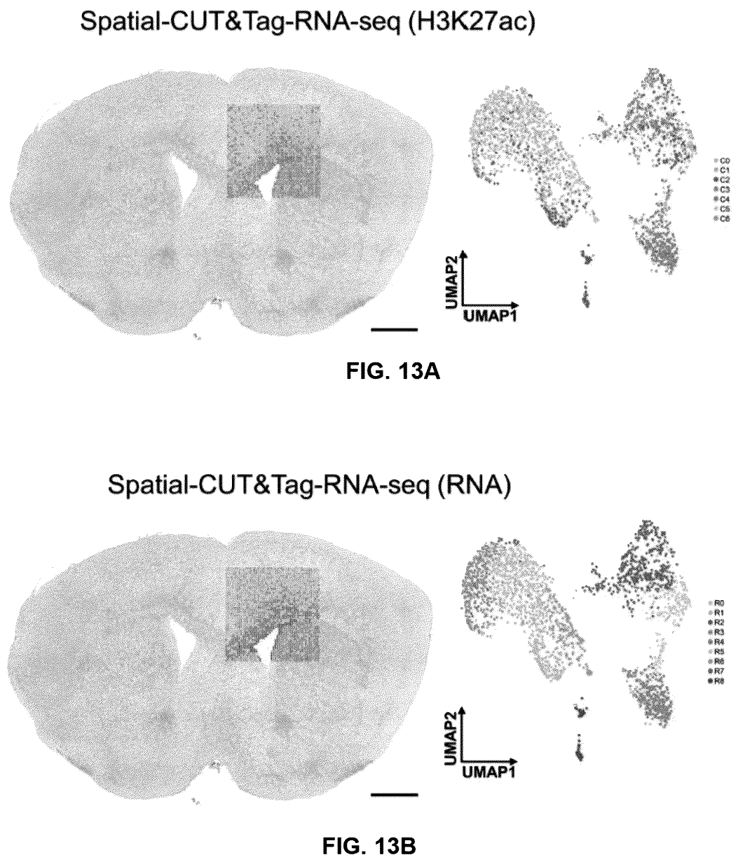

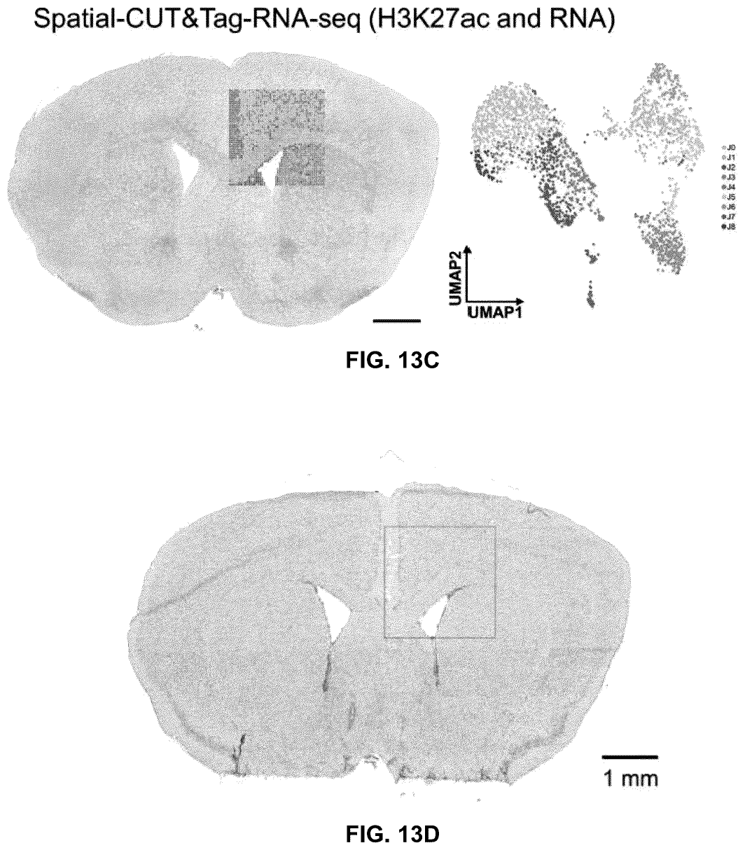

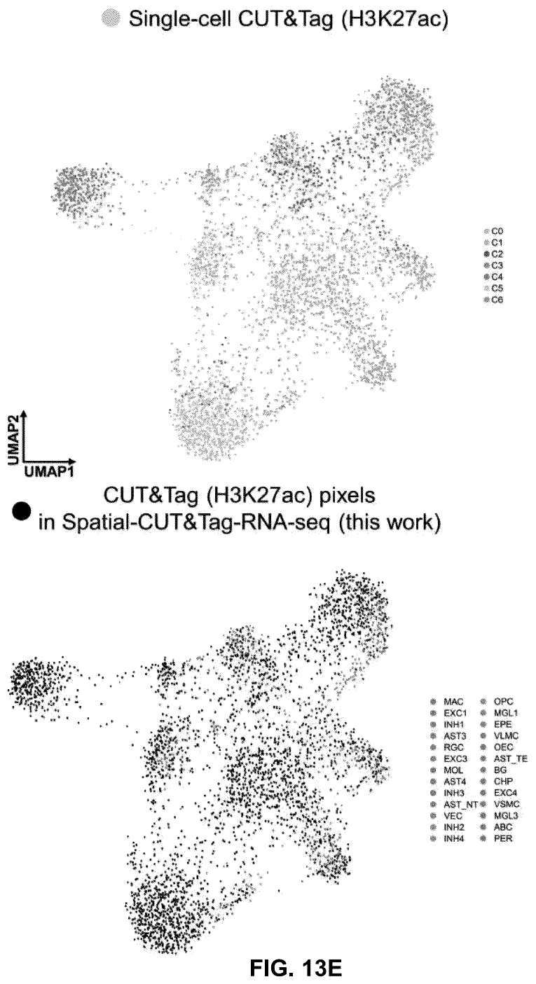

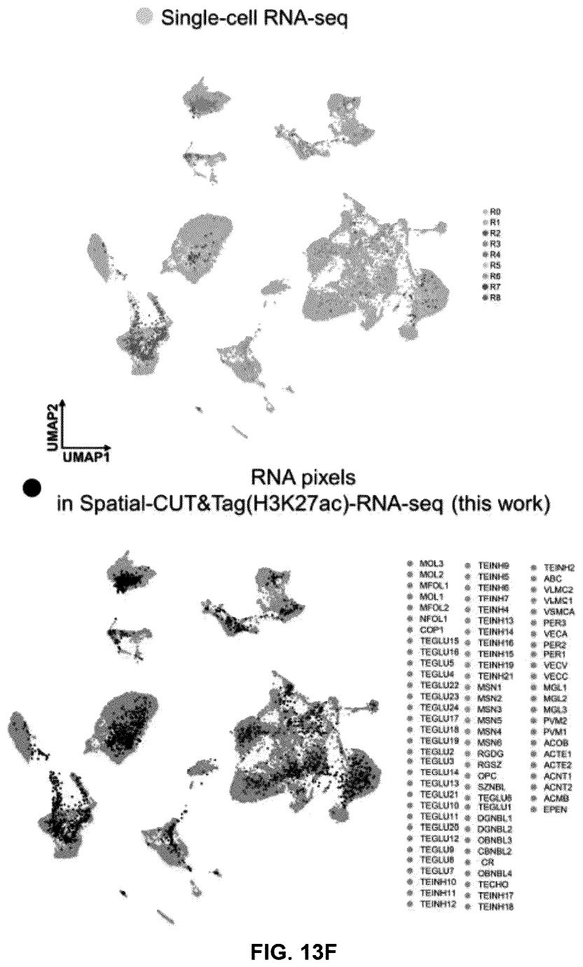

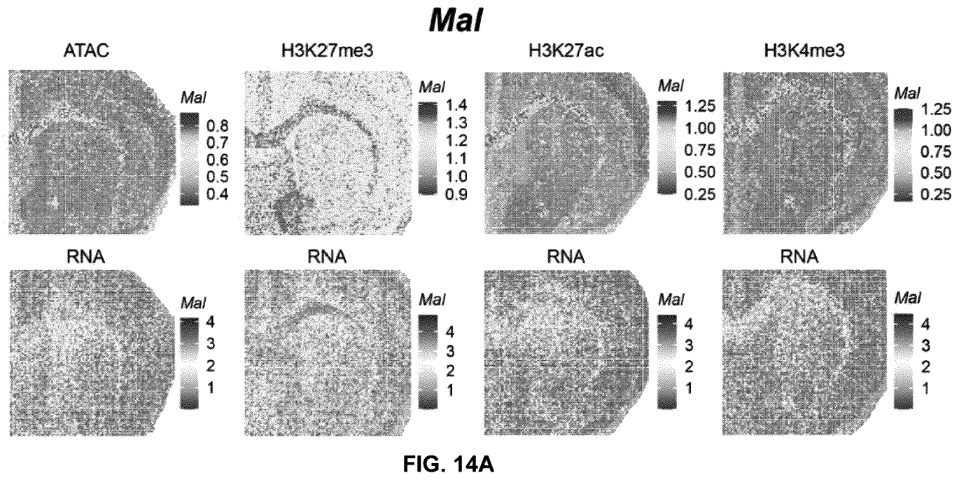

Integration and co-embedding of spatial-CUT&Tag and scCUT&Tag data (Bartosovic, 2021, Nature Biotechnology, 39:825-835; Bartosovic, 2022, Nat Biotechnol, doi: 10.1038/s41587-022-01535-4. Online ahead of print) (FIG. 3D-FIG. 3E and FIG. 12A) revealed the epigenetic states in the data for H3K27me3, H3K27ac, and H3K4me3 agreed with the corresponding projection in scCUT&Tag. Integration with the corresponding juvenile CNS scRNA-seq atlas (Zeisel, 2018, Cell, 174:999-1014) allowed for label transfer to assign transcriptional cell types to the spatial location of epigenetic identities/states (FIG. 12G-FIG. 12H, bottom) such as an enrichment of MOL within the corpus callosum, a thin layer of ependymal cells (EPEN) in the lateral ventricle, excitatory neurons (TEGLU) in cerebral cortex, and MSN in the striatum. Paired RNA data was integrated with with scRNA-seq atlas (Zeisel, 2018, Cell, 174:999-1014) to identify dominant cell types via label transfer (FIG. 3F and FIG. 12B-FIG. 12H). MOL, a thin layer of EPEN, MSN, and TEGLU were enriched in the same spatial regions identified by CUT&Tag (H3K27ac and H3K4me3) (FIG. 12G-FIG. 12H, top). In particular, for spatial H3K27me3, while integration with scCUT&Tag could not clearly reveal the identity of several clusters in the epigenomic modalities (cluster C0, C1, and C3), label transfer of scRNA-seq data with the paired RNA data from the same tissue section clearly revealed the cell identities in these clusters (FIG. 3A, FIG. 3D, and FIG. 12F), highlighting the power of combining CUT&Tag and RNA-seq (spatial-CUT&Tag-RNA-seq) in the same tissue section to identify cell types and states. To directly infer the interactions between genome-wide gene expression and the corresponding enhancers across all clusters, a total of 19,468 significant peak-to-gene linkages from spatial-CUT&Tag (H3K27ac)-RNA-seq were identified (FIG. 22A-FIG. 22B). A biological replicate was also performed on P21 mouse brain (FIG. 13, 20 μm pixel size, 50×50 barcodes).

### Region-Specific Gene Expression Regulation

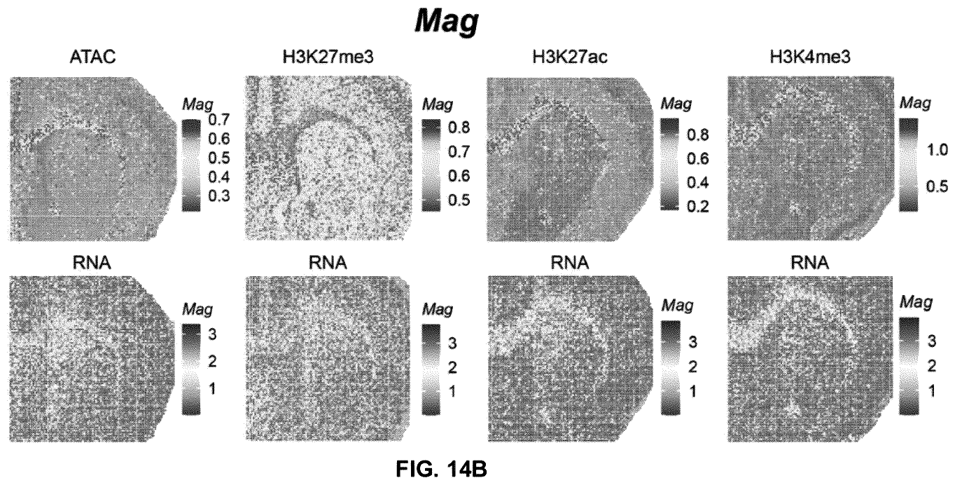

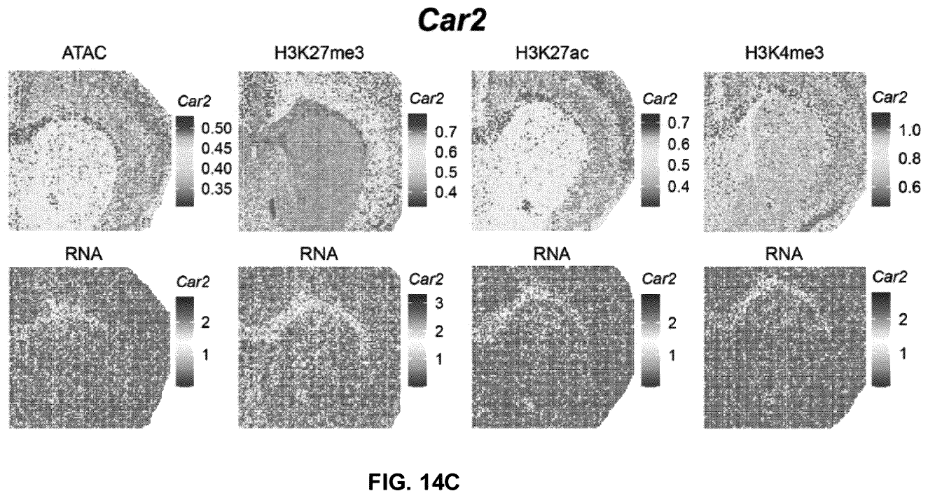

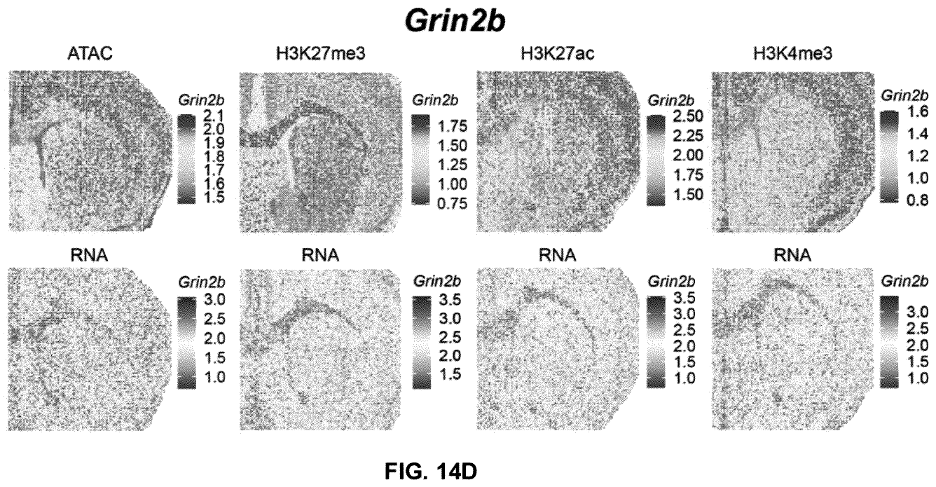

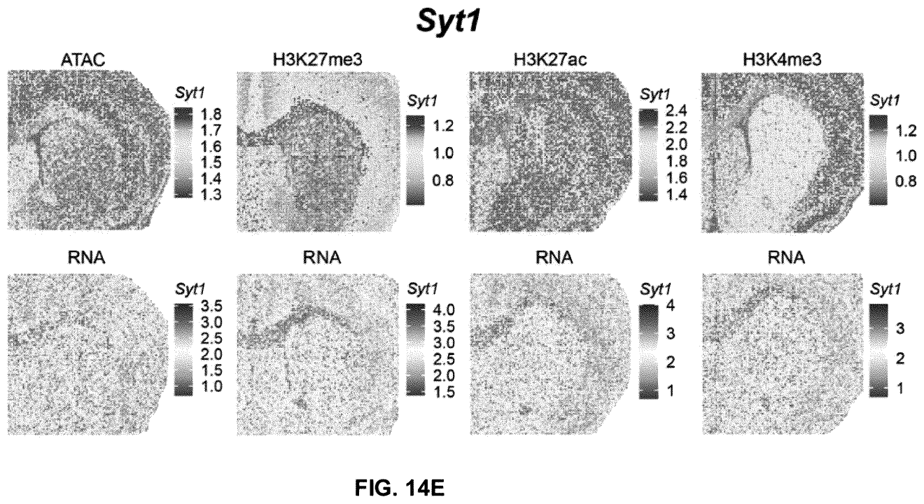

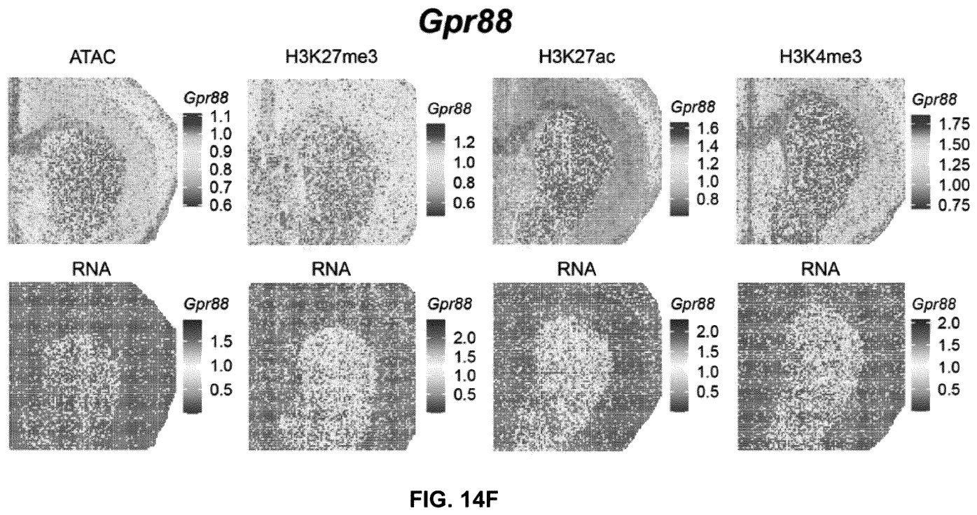

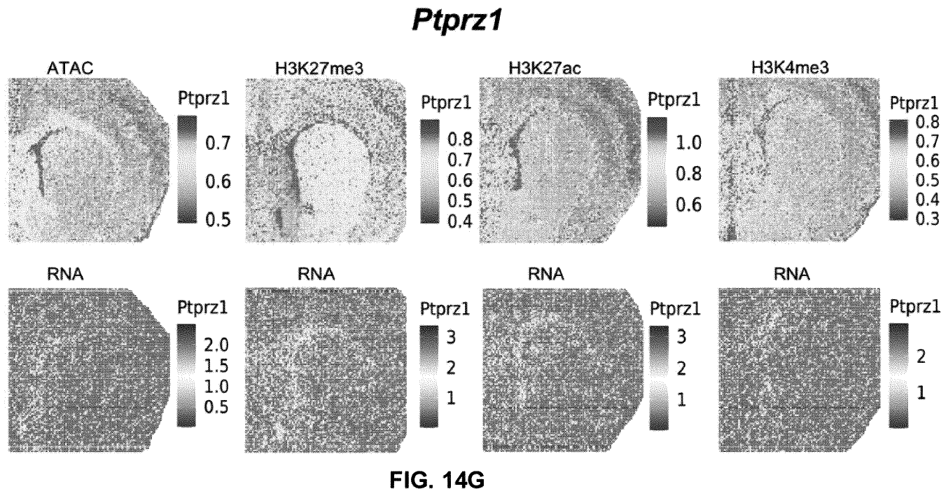

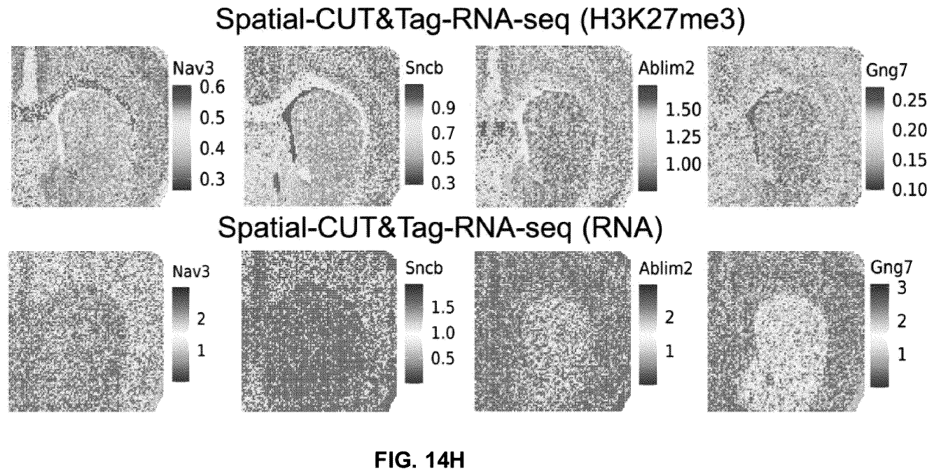

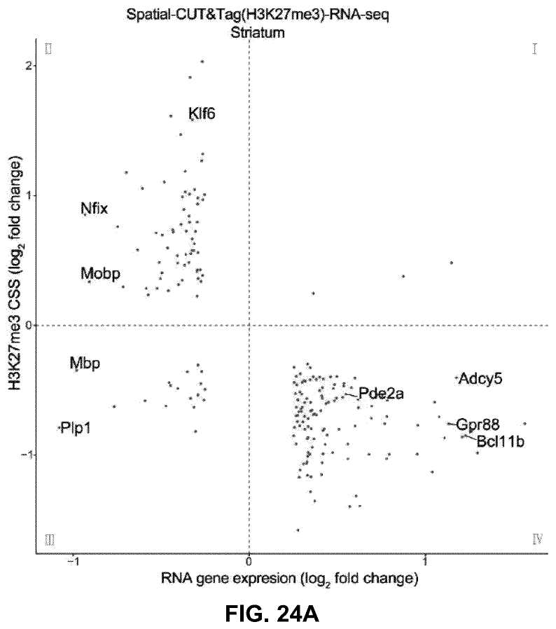

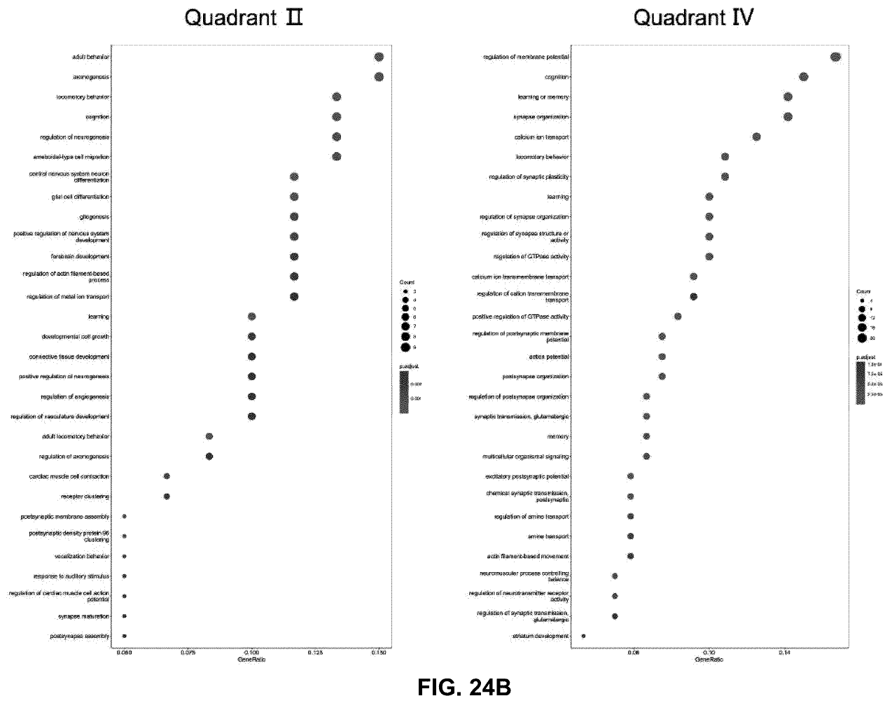

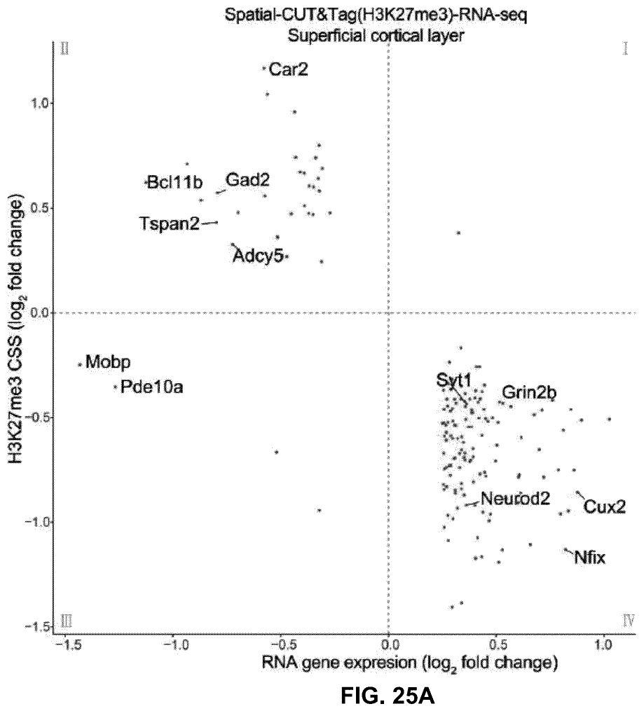

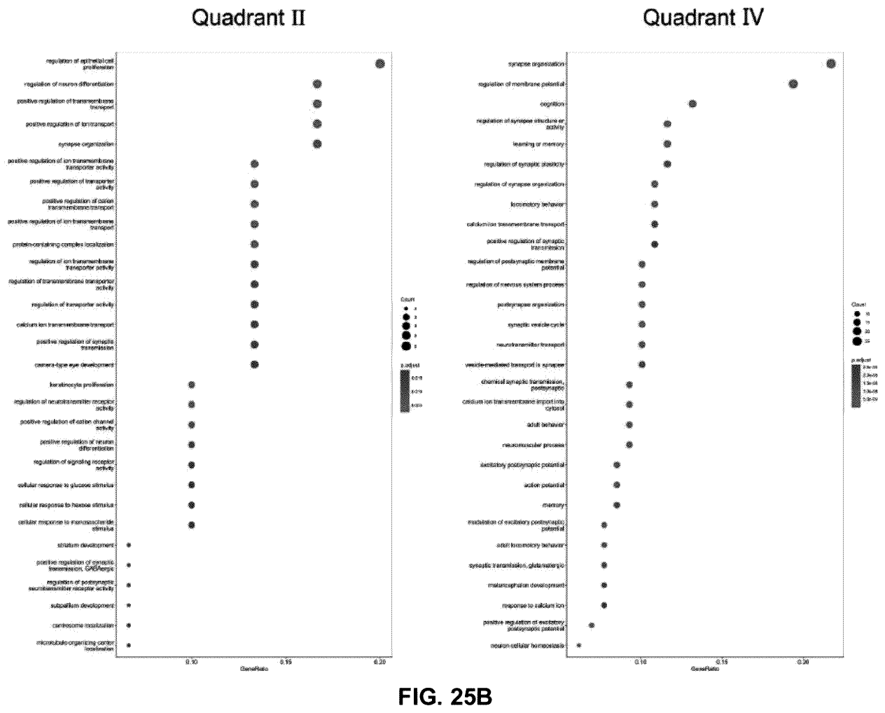

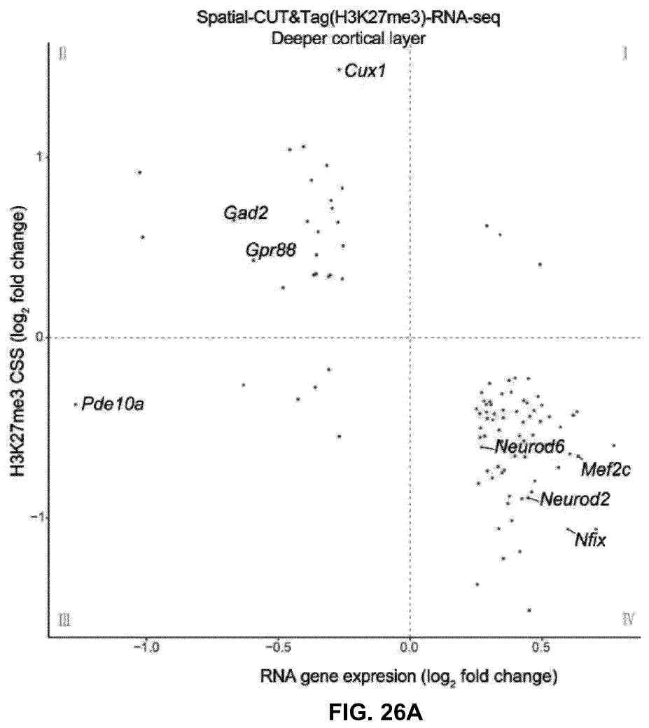

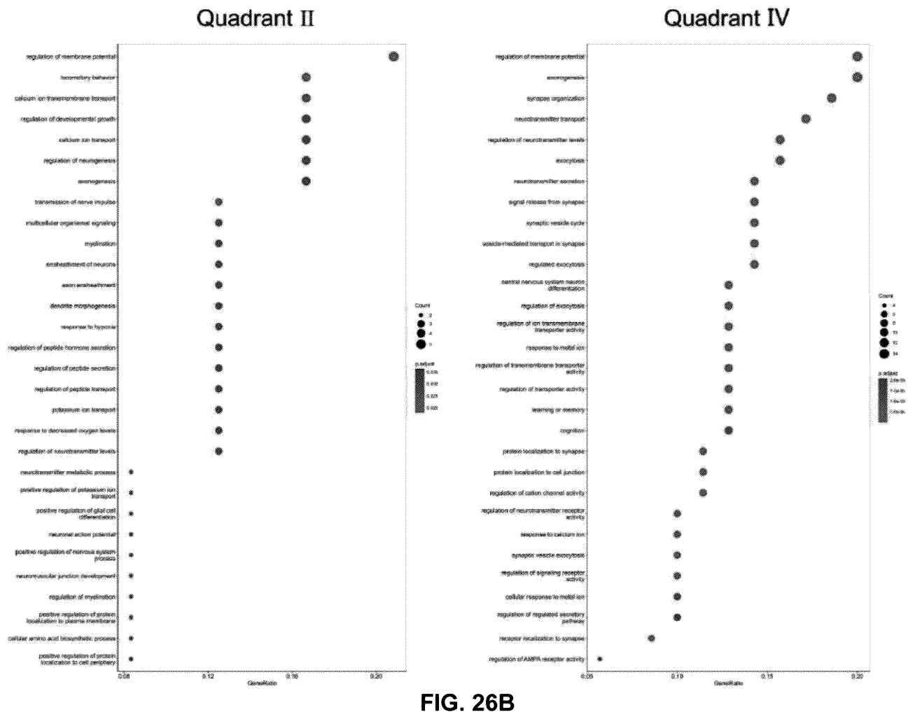

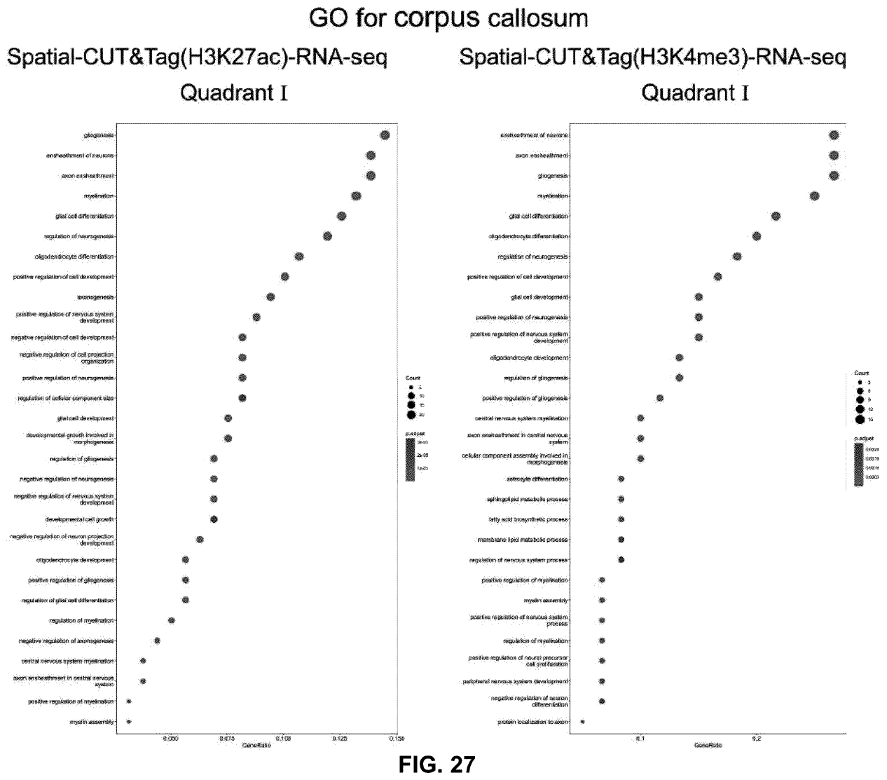

To further understand the spatial epigenetic regulation of gene expression genome-wide, the GAS or CSS obtained from ATAC, CUT&Tag (H3K27me3, H3K27ac, H3K4me3) were compared with the corresponding RNA expression in all major tissue regions (FIG. 4A-FIG. 4C and FIG. 24A, FIG. 25A, and FIG. 26A). For example, in oligodendrocyte-abundant corpus callosum, a robust anti-correlation between H3K27me3 and RNA was observed (FIG. 4A). Genes like Mal, Mag, and Car2 with low CSS and high RNA expression correspond to GO biological processes as myelin and regulation of oligodendrocyte differentiation (FIG. 14A-FIG. 14C and FIG. 23, GO for quadrant IV). In contrast, genes like Grin2b and Syt1 with high CSS and low RNA expression are related to neuronal processes as synaptic transmission and neurotransmitter release (FIG. 14D-FIG. 14E and FIG. 23, GO for quadrant II). These results are in accordance with ATAC/H3K27me3/H3K27ac Nano-CUT&Tag analysis (Bartosovic, 2022, Nat Biotechnol, doi: 10.1038/s41587-022-01535-4. Online ahead of print) of the oligodendroglial differentiation in the juvenile brain, which revealed two waves of H3K27me3 repression during this process (Bartosovic, 2022, Nat Biotechnol, doi: 10.1038/s41587-022-01535-4. Online ahead of print). Interestingly, a small subset of genes was found (FIG. 4A and FIG. 23, GO for quadrant I) with higher levels of both H3K27me3 and RNA in the corpus callosum, such as Ptprz1, a marker of oligodendrocyte precursor cells (OPCs) (Marques, 2016, Science, 352:1326-1329; Marques 2018, Developmental Cell, 46:504-517) (FIG. 14G), which might indicate transcriptional poising of some of these genes. It could be due in part to the presence in proximity of both OPCs (expressing Ptprz1) and mature oligodendrocytes (where Ptprz1 is repressed) in the corpus callosum. A similar correlation analysis for RNA and H3K27me3 in other regions of the juvenile brain, including the striatum, superficial and deeper cortical layers (FIG. 24A, FIG. 25A, and FIG. 26A) was performed. A strong anti-correlation with a cohort of genes involved in neuronal processes being activated or repressed in a region-specific manner was also observed. For instance, in the superficial cortical layer, GABAergic regulation of synaptic transmission was enriched in H3K27me3, while glutamatergic synapse transmission genes had high expression and low H3K27me3 (FIG. 24B, FIG. 25B, and FIG. 26B, GOs for striatum, superficial and deeper cortical layers). Interestingly, in contrast to corpus callosum, only a limited number of genes positive for both H3K27me3 and RNA were found in these regions. Despite a general anti-correlation between RNA expression and H3K27me3 deposition, regional differences were also observed (highlighted in blue columns in the upset plot in FIG. 4D). Nav3 and Sncb with low levels of H3K27me3 both in the cortex and striatum were expressed in the former but not the latter (FIG. 14H). The opposite pattern was observed for genes as Ablim2 and Gng7 (FIG. 14H). Car2, a marker gene expressed in oligodendrocytes and abundant in the corpus callosum, presented H3K27me3 occupancy in the cortical layers, in contrast to the corpus callosum and surprisingly the striatum (FIG. 14C). Thus, mechanisms other than Polycomb-mediated H3K27me3 deposition might be involved in the transcriptional repression of these genes in different areas of the CNS.

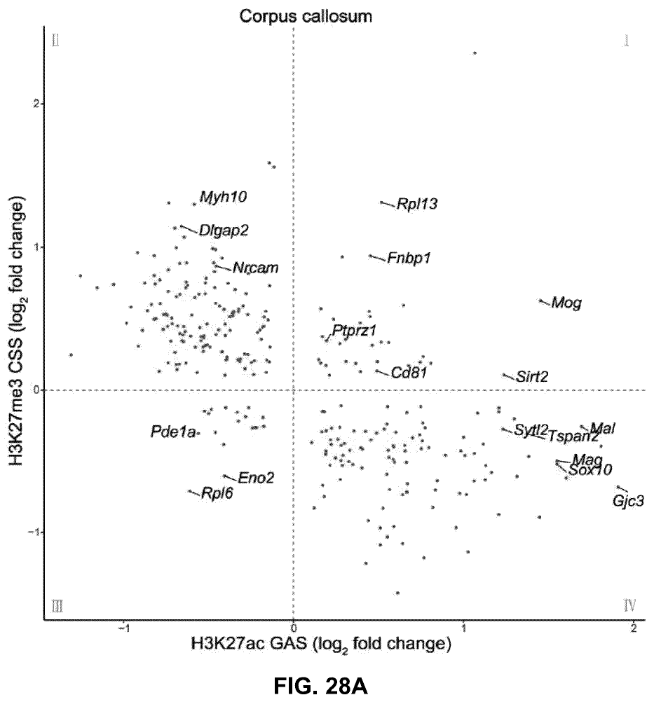

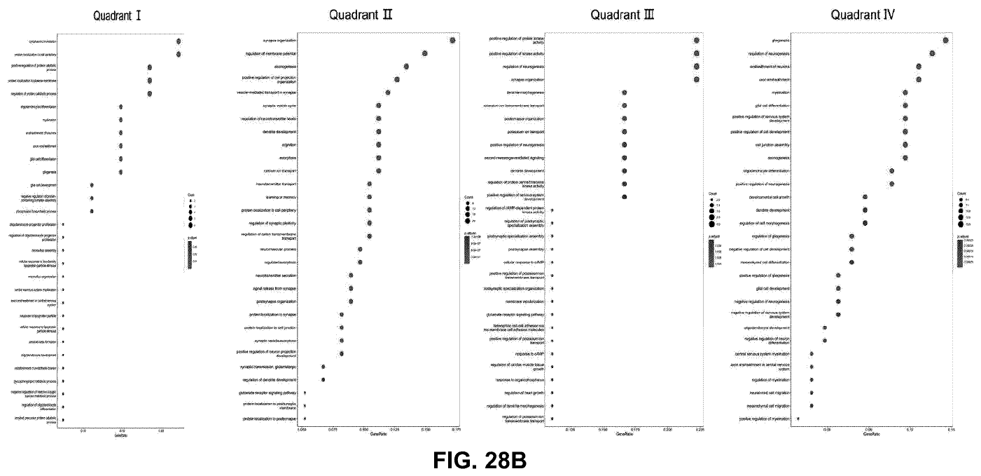

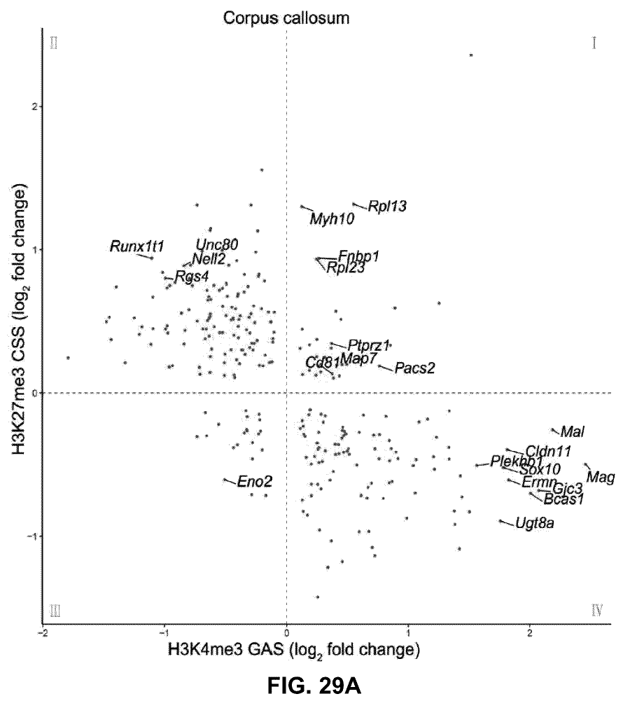

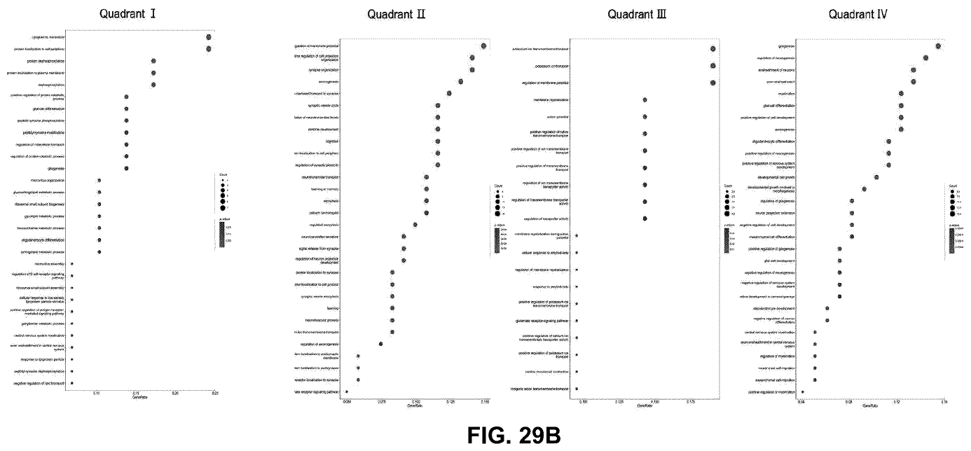

In contrast to H3K27me3, a robust correlation between RNA and activating marks H3K4me3 or H3K27ac was observed in the corpus callosum, with genes highly expressed and with high deposition in these two modalities regulating processes in oligodendroglia (FIG. 4B, FIG. 4C, and FIG. 27, GO for H3K27ac quadrant I and H3K4me3 quadrant I). For instance, Mal showed the highest activity or expression in the corpus callosum for ATAC, H3K27ac, H3K4me3 and the paired RNA (FIG. 14A). Gpr88, a marker gene of MSN, was enriched in striatum for ATAC, H3K27ac, and H3K4me3, whereas low CSS in striatum for H3K27me3 (FIG. 14F). Furthermore, the collective regulation among different histone modifications in the corpus callosum was examined (FIG. 4E-FIG. 4G and FIG. 28-FIG. 29). In general, H3K4me3 and H3K27ac showed high correlations with their signals anti-correlated with H3K27me3 (FIG. 4E-FIG. 4F). Interestingly, H3K4me3 or H3K27ac and H3K27me3 co-occupancy in several gene loci in this tissue region was also observed (FIG. 4G and FIG. 28-FIG. 29). These genes are involved in oligodendrocyte differentiation and myelination and other processes such as protein catabolismand localization (FIG. 28-FIG. 29). Interestingly, some of these genes such as Ptprz1 and Fnbp1 also presented higher levels of H3K27me3 and RNA (FIG. 4A). As mentioned before, this potential H3K4me3/H3K27me3 bivalency might reflect transcriptional poising.

### Spatial Co-Mapping of Human Brain

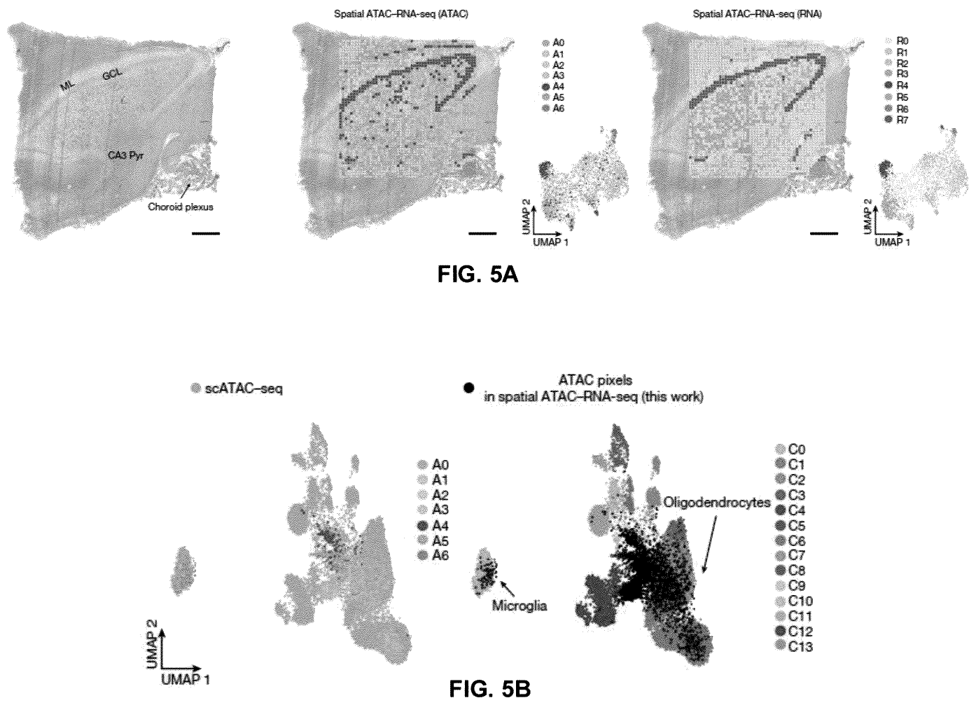

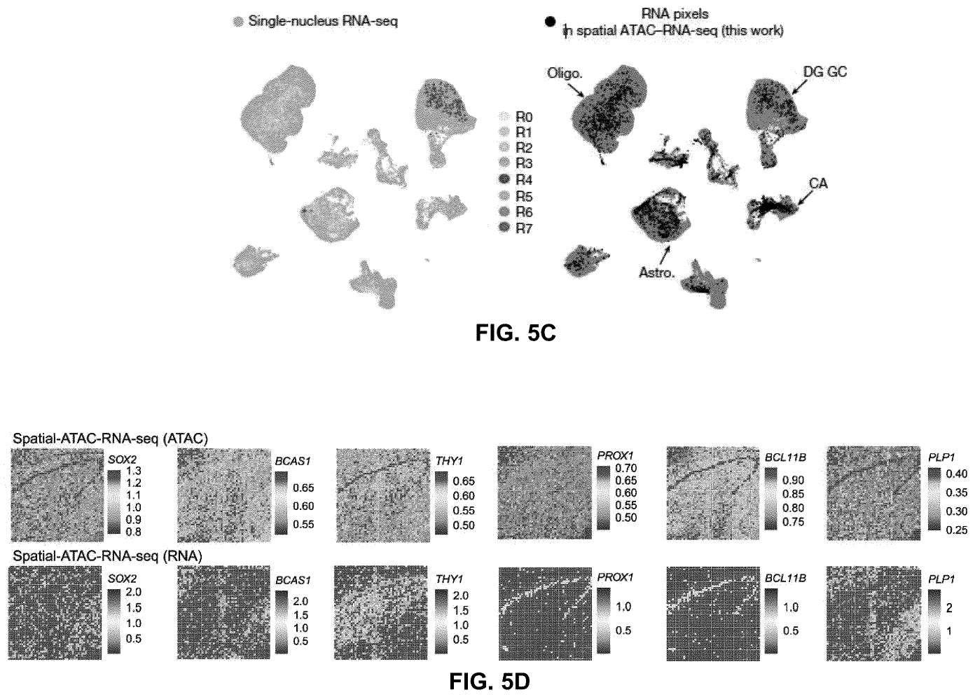

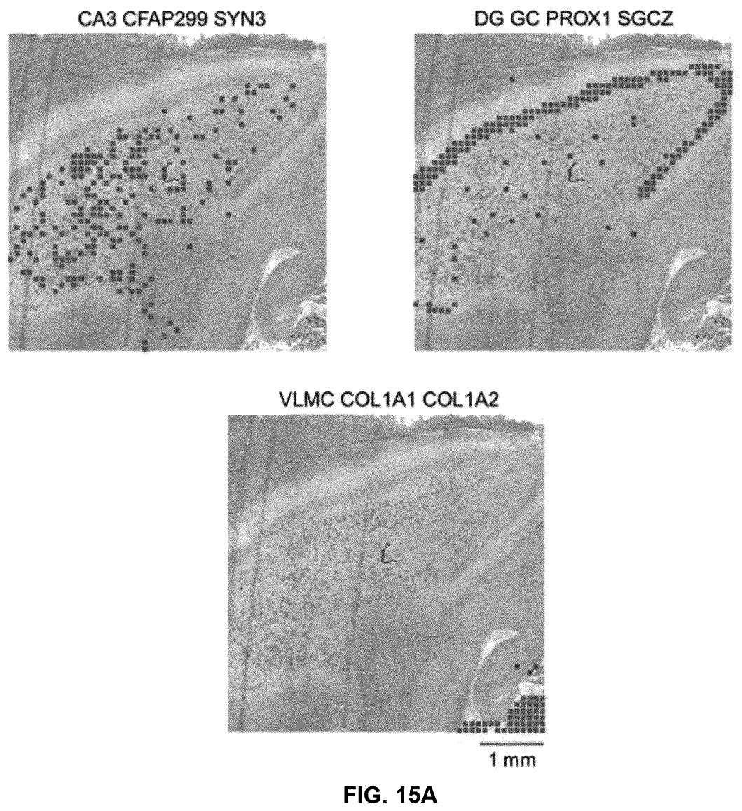

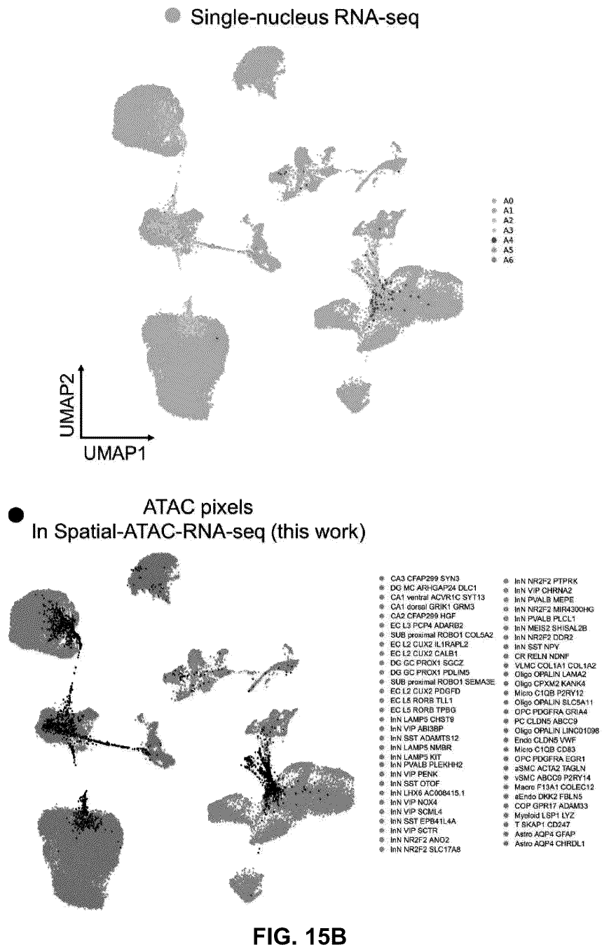

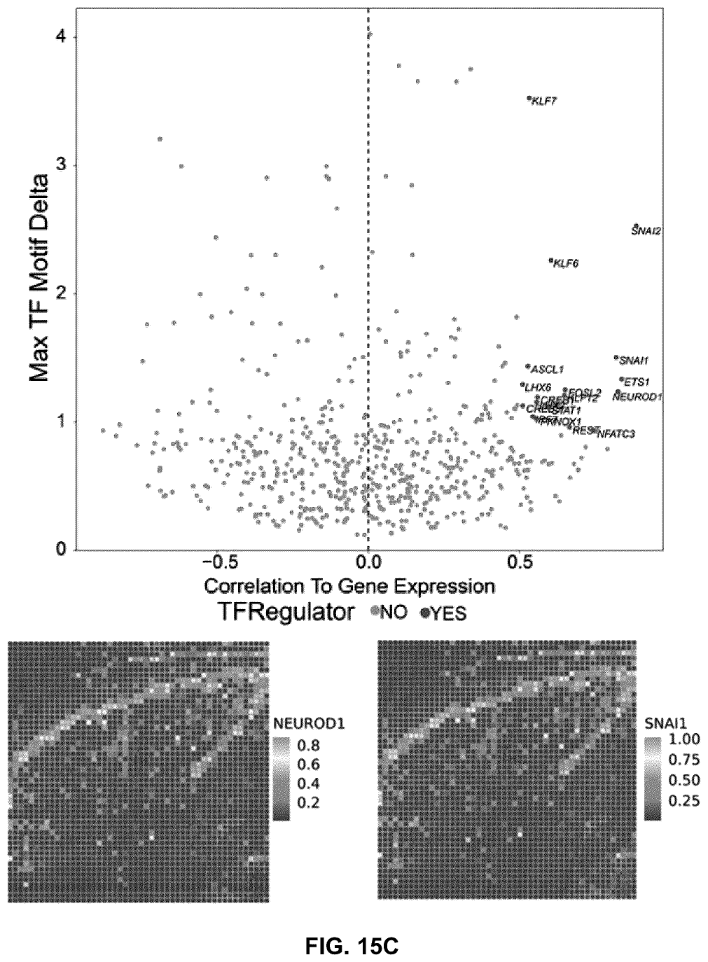

Spatial-ATAC-RNA-seq was performed on adult (31-year-old male) human brain hippocampal formation, which is a complex brain region involved in cognitive functions and diseases like major depression disease (MDD) (Otte, 2016, Nature Reviews Disease Primers, 2:16065) and Alzheimer's disease (AD) (Wagner, 2022, Nature, 612:123-131). 7 and 8 major clusters for ATAC and RNA were identified, respectively, and the spatial pattern agreed with the major anatomical landmarks (FIG. 5A) (Deng, 2022, Nature, 609:375-383), including specific marker genes (FIG. 5D). The ATAC cluster A4 represents the granule cell layer (GCL) (THY1, BCL11B) and cluster A6 corresponds to choroid plexus (FIG. 5A and FIG. 5D). TF motifs (NEUROD1, SNAI1) and their spatial patterns were visualized in different tissue regions and the positive TF regulators were identified (FIG. 15B-FIG. 15C). For the RNA data, distinct clusters with unique marker genes (FIG. 5A and FIG. 5D) such as PROX1 and BCL11B enriched in cluster R4 (GCL) were also detected.

ATAC data and scATAC-seq from human brain samples (Corces, 2020, Nat Genet, 52:1158-1168) (FIG. 5B) were integrated with RNA data with adult human brain snRNA-seq (Franjic, 2022, Neuron, 110:452-469) to reveal the dominant cell identities and states. Cell types identified by snRNA-seq (Franjic, 2022, Neuron, 110:452-469) were assigned to each cluster via label transfer (FIG. 5C and FIG. 15A). The granule cells were detected clearly in the GCL, the cornu ammonis (CA) neurons were enriched in CA3-4 regions, and the vascular and leptomeningeal cells (VLMC) were strongly distinguished in choroid plexus (ChPx) as opposed to other regions.

In general, both ATAC and RNA can readily resolve all major tissue features in this region but spatial co-sequencing of epigenome and transcriptome can provide new insights into the dynamic gene regulation mechanism which cannot be realized by single modalities. For example, PROX1, a signature gene defining the granule neuron identity during pyramidal neuron fate selection (La Manno, 2018, Nature, 110:452-469), is indeed highly expressed in GCL but showed modest chromatin accessibility (FIG. 5D). This might be attributed to a minimal demand to synthesize new PROX1 transcripts in postmitotic mature granule cells and thus not required to maintain an active open chromatin state.

TABLE 1
Summary of metrics for ATAC and RNA in spatial-ATAC-RNA-seq for all the samples.

| | | | E13 | P22 | P21 mouse | P21 mouse | |
|---|---|---|---|---|---|---|---|
| | | | mouse | mouse | brain | brain | Human |
| | | | embryo | brain | (replica 1, | (replica 2, | brain |
| | | | (50 | (100 | 50 | 50 | (50 |
| | | | barcodes, | barcodes, | barcodes, | barcodes, | barcodes, |
| | | | 50 μm | 20 μm | 20 μm | 20 μm | 50 μm |
| | | | pixel size) | pixel size) | pixel size) | pixel size) | pixel size) |
| Spatial- | ATAC | Number of | 18,079 | 14,284 | 10,857 | 14,385 | 9,898 |
| ATAC- | | Unique | | | | | |
| RNA-seq | | fragments | | | | | |
| | | TSS | 16% | 19% | 20% | 19% | 15% |
| | | fragments | | | | | |
| | | FRiP | 11% | 26% | 24% | 26% | 11% |
| | | Mitochondrial | 0.96% | 4.6% | 9% | 8.4% | 20% |
| | | fragments | | | | | |
| | RNA | Average | 1,255 | 1,073 | 1,005 | 1,600 | 1,200 |
| | | number of | | | | | |
| | | genes per | | | | | |
| | | pixel | | | | | |
| | | Average | 3,603 | 2,358 | 2,391 | 3,811 | 2,809 |
| | | number of | | | | | |
| | | UMIs per | | | | | |
| | | pixel | | | | | |
| | | Number of | 20,900 | 22,914 | 19,859 | 20,046 | 29,293 |
| | | unique | | | | | |
| | | genes | | | | | |
| | | present | | | | | |
| | | Pixels on | 2,187 | 9,215 | 2,373 | 2,498 | 2,500 |
| | | tissue | | | | | |

TABLE 2
Summary of metrics for CUT&Tag and RNA in spatial-CUT&Tag-RNA-seq for all the samples.

| | | | | P22 | P22 | P21 mouse | P21 mouse |
|---|---|---|---|---|---|---|---|
| | | | P22 mouse | mouse | mouse | brain | brain |
| | | | brain | brain | brain | (H3K27ac, | (H3K27ac, |
| | | | (H3K27me3) | (H3K27ac) | (H3K4me3) | replica 1) | replica 2) |
| | | | (100 | (100 | (100 | (50 | (50 |
| | | | barcodes, | barcodes, | barcodes, | barcodes, | barcodes, |
| | | | 20 μm | 20 μm | 20 μm | 20 μm | 20 μm |
| | | | pixel size) | pixel size) | pixel size) | pixel size) | pixel size) |
| Spatial- | CUT&Tag | Number of | 10,644 | 10,002 | 2,507 | 4,756 | 5,022 |
| CUT&Tgag- | RNA | Unique | | | | | |
| RNA-seq | | fragments | | | | | |
| | | TSS | 12% | 17% | 67% | 19% | 20% |
| | | fragments | | | | | |
| | | FRiP | 12% | 21% | 54% | 19% | 18% |
| | | Mitochondrial | 0.2% | 0.3% | 3.6% | 0.1% | 0.02% |
| | | fragments | | | | | |
| | | Average | 2,011 | 1,513 | 1,329 | 1,145 | 752 |
| | | number of | | | | | |
| | | genes per | | | | | |
| | | pixel | | | | | |
| | | Average | 4,734 | 3,580 | 2,885 | 2,938 | 1,890 |
| | | number of | | | | | |
| | | UMIs per | | | | | |
| | | pixel | | | | | |
| | | Number of | 25,881 | 23,415 | 22,731 | 19,831 | 18,718 |
| | | unique | | | | | |
| | | genes | | | | | |
| | | present | | | | | |
| | | Pixels on | 9,752 | 9,370 | 9,548 | 2,387 | 2,499 |
| | | tissue | | | | | |

TABLE 3
DNA oligos used for PCR and preparation of sequencing library.

| | | |
|---|---|---|
| RT | SEQ ID | /5Phos/CATCGGCGTACGACTNNNNNNNNNN/iBiodT/TTT |
| primer | NO: 201 | TTTTTTTTTTTTVN |
| Ligation | SEQ ID | AGTCGTACGCCGATGCGAAACATCGGCCAC |
| linker 1 | NO: 202 | |
| Ligation | SEQ ID | CGAATGCTCTGGCCTCTCAAGCACGTGGAT |
| linker 2 | NO: 203 | |
| PCR | SEQ ID | CAAGCGTTGGCTTCTCGCATCT |
| Primer 1 | NO: 204 | |
| PCR | SEQ ID | AAGCAGTGGTATCAACGCAGAGT |
| Primer 2 | NO: 205 | |
| N501 | SEQ ID | AATGATACGGCGACCACCGAGATCTACACTAGATCGCT |
| | NO: 206 | CGTCGGCAGCGTCAGATGTGTATAAGAGACAG |
| N701 | SEQ ID | CAAGCAGAAGACGGCATACGAGATTCGCCTTAGTCTC |
| | NO: 207 | GTGGGCTCGGAGATGTGTATAAGAGACAGCAAGCGTT |
| | | GGCTTCTCGCATCT |
| N702 | SEQ ID | CAAGCAGAAGACGGCATACGAGATCTAGTACGGTCTC |
| | NO: 208 | GTGGGCTCGGAGATGTGTATAAGAGACAGCAAGCGTT |
| | | GGCTTCTCGCATCT |
| N703 | SEQ ID | CAAGCAGAAGACGGCATACGAGATTTCTGCCTGTCTCG |
| | NO: 209 | TGGGCTCGGAGATGTGTATAAGAGACAGCAAGCGTTG |
| | | GCTTCTCGCATCT |
| N704 | SEQ ID | CAAGCAGAAGACGGCATACGAGATGCTCAGGAGTCTC |
| | NO: 210 | GTGGGCTCGGAGATGTGTATAAGAGACAGCAAGCGTT |
| | | GGCTTCTCGCATCT |
| N705 | SEQ ID | CAAGCAGAAGACGGCATACGAGATAGGAGTCCGTCTC |
| | NO: 211 | GTGGGCTCGGAGATGTGTATAAGAGACAGCAAGCGTT |
| | | GGCTTCTCGCATCT |
| N706 | SEQ ID | CAAGCAGAAGACGGCATACGAGATCATGCCTAGTCTC |
| | NO: 212 | GTGGGCTCGGAGATGTGTATAAGAGACAGCAAGCGTT |
| | | GGCTTCTCGCATCT |
| N707 | SEQ ID | CAAGCAGAAGACGGCATACGAGATGTAGAGAGGTCTC |
| | NO: 213 | GTGGGCTCGGAGATGTGTATAAGAGACAGCAAGCGTT |
| | | GGCTTCTCGCATCT |

TABLE 4
DNA barcode A sequences.

| Barcode | SEQ ID | |
|---|---|---|
| A | NO: | Sequence |
| Barcode | SEQ ID | /5Phos/AGGCCAGAGCATTCGAACGTGATGTGGCCGAT |
| A-1 | NO: 1 | GTTTCG |
| Barcode | SEQ ID | /5Phos/AGGCCAGAGCATTCGAAACATCGGTGGCCGAT |
| A-2 | NO: 2 | GTTTCG |
| Barcode | SEQ ID | /5Phos/AGGCCAGAGCATTCGATGCCTAAGTGGCCGAT |
| A-3 | NO: 3 | GTTTCG |
| Barcode | SEQ ID | /5Phos/AGGCCAGAGCATTCGAGTGGTCAGTGGCCGAT |
| A-4 | NO: 4 | GTTTCG |
| Barcode | SEQ ID | /5Phos/AGGCCAGAGCATTCGACCACTGTGTGGCCGAT |
| A-5 | NO: 5 | GTTTCG |
| Barcode | SEQ ID | /5Phos/AGGCCAGAGCATTCGACATTGGCGTGGCCGAT |
| A-6 | NO: 6 | GTTTCG |
| Barcode | SEQ ID | /5Phos/AGGCCAGAGCATTCGCAGATCTGGTGGCCGAT |
| A-7 | NO: 7 | GTTTCG |
| Barcode | SEQ ID | /5Phos/AGGCCAGAGCATTCGCATCAAGTGTGGCCGAT |
| A-8 | NO: 8 | GTTTCG |
| Barcode | SEQ ID | /5Phos/AGGCCAGAGCATTCGCGCTGATCGTGGCCGAT |
| A-9 | NO: 9 | GTTTCG |
| Barcode | SEQ ID | /5Phos/AGGCCAGAGCATTCGACAAGCTAGTGGCCGAT |
| A-10 | NO: 10 | GTTTCG |
| Barcode | SEQ ID | /5Phos/AGGCCAGAGCATTCGCTGTAGCCGTGGCCGAT |
| A-11 | NO: 11 | GTTTCG |
| Barcode | SEQ ID | /5Phos/AGGCCAGAGCATTCGAGTACAAGGTGGCCGAT |
| A-12 | NO: 12 | GTTTCG |
| Barcode | SEQ ID | /5Phos/AGGCCAGAGCATTCGAACAACCAGTGGCCGAT |
| A-13 | NO: 13 | GTTTCG |
| Barcode | SEQ ID | /5Phos/AGGCCAGAGCATTCGAACCGAGAGTGGCCGAT |
| A-14 | NO: 14 | GTTTCG |
| Barcode | SEQ ID | /5Phos/AGGCCAGAGCATTCGAACGCTTAGTGGCCGAT |
| A-15 | NO: 15 | GTTTCG |
| Barcode | SEQ ID | /5Phos/AGGCCAGAGCATTCGAAGACGGAGTGGCCGAT |
| A-16 | NO: 16 | GTTTCG |
| Barcode | SEQ ID | /5Phos/AGGCCAGAGCATTCGAAGGTACAGTGGCCGAT |
| A-17 | NO: 17 | GTTTCG |
| Barcode | SEQ ID | /5Phos/AGGCCAGAGCATTCGACACAGAAGTGGCCGAT |
| A-18 | NO: 18 | GTTTCG |
| Barcode | SEQ ID | /5Phos/AGGCCAGAGCATTCGACAGCAGAGTGGCCGAT |
| A-19 | NO: 19 | GTTTCG |
| Barcode | SEQ ID | /5Phos/AGGCCAGAGCATTCGACCTCCAAGTGGCCGAT |
| A-20 | NO: 20 | GTTTCG |
| Barcode | SEQ ID | /5Phos/AGGCCAGAGCATTCGACGCTCGAGTGGCCGAT |
| A-21 | NO: 21 | GTTTCG |
| Barcode | SEQ ID | /5Phos/AGGCCAGAGCATTCGACGTATCAGTGGCCGAT |
| A-22 | NO: 22 | GTTTCG |
| Barcode | SEQ ID | /5Phos/AGGCCAGAGCATTCGACTATGCAGTGGCCGAT |
| A-23 | NO: 23 | GTTTCG |
| Barcode | SEQ ID | /5Phos/AGGCCAGAGCATTCGAGAGTCAAGTGGCCGAT |
| A-24 | NO: 24 | GTTTCG |
| Barcode | SEQ ID | /5Phos/AGGCCAGAGCATTCGAGATCGCAGTGGCCGAT |
| A-25 | NO: 25 | GTTTCG |
| Barcode | SEQ ID | /5Phos/AGGCCAGAGCATTCGAGCAGGAAGTGGCCGAT |
| A-26 | NO: 26 | GTTTCG |
| Barcode | SEQ ID | /5Phos/AGGCCAGAGCATTCGAGTCACTAGTGGCCGAT |
| A-27 | NO: 27 | GTTTCG |
| Barcode | SEQ ID | /5Phos/AGGCCAGAGCATTCGATCCTGTAGTGGCCGAT |
| A-28 | NO: 28 | GTTTCG |
| Barcode | SEQ ID | /5Phos/AGGCCAGAGCATTCGATTGAGGAGTGGCCGAT |
| A-29 | NO: 29 | GTTTCG |
| Barcode | SEQ ID | /5Phos/AGGCCAGAGCATTCGCAACCACAGTGGCCGAT |
| A-30 | NO: 30 | GTTTCG |
| Barcode | SEQ ID | /5Phos/AGGCCAGAGCATTCGGACTAGTAGTGGCCGAT |
| A-31 | NO: 31 | GTTTCG |
| Barcode | SEQ ID | /5Phos/AGGCCAGAGCATTCGCAATGGAAGTGGCCGAT |
| A-32 | NO: 32 | GTTTCG |
| Barcode | SEQ ID | /5Phos/AGGCCAGAGCATTCGCACTTCGAGTGGCCGAT |
| A-33 | NO: 33 | GTTTCG |
| Barcode | SEQ ID | /5Phos/AGGCCAGAGCATTCGCAGCGTTAGTGGCCGAT |
| A-34 | NO: 34 | GTTTCG |
| Barcode | SEQ ID | /5Phos/AGGCCAGAGCATTCGCATACCAAGTGGCCGAT |
| A-35 | NO: 35 | GTTTCG |
| Barcode | SEQ ID | /5Phos/AGGCCAGAGCATTCGCCAGTTCAGTGGCCGAT |
| A-36 | NO: 36 | GTTTCG |
| Barcode | SEQ ID | /5Phos/AGGCCAGAGCATTCGCCGAAGTAGTGGCCGAT |
| A-37 | NO: 37 | GTTTCG |
| Barcode | SEQ ID | /5Phos/AGGCCAGAGCATTCGCCGTGAGAGTGGCCGAT |
| A-38 | NO: 38 | GTTTCG |
| Barcode | SEQ ID | /5Phos/AGGCCAGAGCATTCGCCTCCTGAGTGGCCGAT |
| A-39 | NO: 39 | GTTTCG |
| Barcode | SEQ ID | /5Phos/AGGCCAGAGCATTCGCGAACTTAGTGGCCGAT |
| A-40 | NO: 40 | GTTTCG |
| Barcode | SEQ ID | /5Phos/AGGCCAGAGCATTCGCGACTGGAGTGGCCGAT |
| A-41 | NO: 41 | GTTTCG |
| Barcode | SEQ ID | /5Phos/AGGCCAGAGCATTCGCGCATACAGTGGCCGAT |
| A-42 | NO: 42 | GTTTCG |
| Barcode | SEQ ID | /5Phos/AGGCCAGAGCATTCGCTCAATGAGTGGCCGAT |
| A-43 | NO: 43 | GTTTCG |
| Barcode | SEQ ID | /5Phos/AGGCCAGAGCATTCGCTGAGCCAGTGGCCGAT |
| A-44 | NO: 44 | GTTTCG |
| Barcode | SEQ ID | /5Phos/AGGCCAGAGCATTCGCTGGCATAGTGGCCGAT |
| A-45 | NO: 45 | GTTTCG |
| Barcode | SEQ ID | /5Phos/AGGCCAGAGCATTCGGAATCTGAGTGGCCGAT |
| A-46 | NO: 46 | GTTTCG |
| Barcode | SEQ ID | /5Phos/AGGCCAGAGCATTCGCAAGACTAGTGGCCGAT |
| A-47 | NO: 47 | GTTTCG |
| Barcode | SEQ ID | /5Phos/AGGCCAGAGCATTCGGAGCTGAAGTGGCCGAT |
| A-48 | NO: 48 | GTTTCG |
| Barcode | SEQ ID | /5Phos/AGGCCAGAGCATTCGGATAGACAGTGGCCGAT |
| A-49 | NO: 49 | GTTTCG |
| Barcode | SEQ ID | /5Phos/AGGCCAGAGCATTCGGCCACATAGTGGCCGAT |
| A-50 | NO: 50 | GTTTCG |
| Barcode | SEQ ID | /5Phos/AGGCCAGAGCATTCGGCGAGTAAGTGGCCGAT |
| A-51 | NO: 51 | GTTTCG |
| Barcode | SEQ ID | /5Phos/AGGCCAGAGCATTCGGCTAACGAGTGGCCGAT |
| A-52 | NO: 52 | GTTTCG |
| Barcode | SEQ ID | /5Phos/AGGCCAGAGCATTCGGCTCGGTAGTGGCCGAT |
| A-53 | NO: 53 | GTTTCG |
| Barcode | SEQ ID | /5Phos/AGGCCAGAGCATTCGGGAGAACAGTGGCCGAT |
| A-54 | NO: 54 | GTTTCG |
| Barcode | SEQ ID | /5Phos/AGGCCAGAGCATTCGGGTGCGAAGTGGCCGAT |
| A-55 | NO: 55 | GTTTCG |
| Barcode | SEQ ID | /5Phos/AGGCCAGAGCATTCGGTACGCAAGTGGCCGAT |
| A-56 | NO: 56 | GTTTCG |
| Barcode | SEQ ID | /5Phos/AGGCCAGAGCATTCGGTCGTAGAGTGGCCGAT |
| A-57 | NO: 57 | GTTTCG |
| Barcode | SEQ ID | /5Phos/AGGCCAGAGCATTCGGTCTGTCAGTGGCCGAT |
| A-58 | NO: 58 | GTTTCG |
| Barcode | SEQ ID | /5Phos/AGGCCAGAGCATTCGGTGTTCTAGTGGCCGAT |
| A-59 | NO: 59 | GTTTCG |
| Barcode | SEQ ID | /5Phos/AGGCCAGAGCATTCGTAGGATGAGTGGCCGAT |
| A-60 | NO: 60 | GTTTCG |
| Barcode | SEQ ID | /5Phos/AGGCCAGAGCATTCGTATCAGCAGTGGCCGAT |
| A-61 | NO: 61 | GTTTCG |
| Barcode | SEQ ID | /5Phos/AGGCCAGAGCATTCGTCCGTCTAGTGGCCGAT |
| A-62 | NO: 62 | GTTTCG |
| Barcode | SEQ ID | /5Phos/AGGCCAGAGCATTCGTCTTCACAGTGGCCGAT |
| A-63 | NO: 63 | GTTTCG |
| Barcode | SEQ ID | /5Phos/AGGCCAGAGCATTCGTGAAGAGAGTGGCCGAT |
| A-64 | NO: 64 | GTTTCG |
| Barcode | SEQ ID | /5Phos/AGGCCAGAGCATTCGTGGAACAAGTGGCCGAT |
| A-65 | NO: 65 | GTTTCG |
| Barcode | SEQ ID | /5Phos/AGGCCAGAGCATTCGTGGCTTCAGTGGCCGAT |
| A-66 | NO: 66 | GTTTCG |
| Barcode | SEQ ID | /5Phos/AGGCCAGAGCATTCGTGGTGGTAGTGGCCGAT |
| A-67 | NO: 67 | GTTTCG |
| Barcode | SEQ ID | /5Phos/AGGCCAGAGCATTCGTTCACGCAGTGGCCGAT |
| A-68 | NO: 68 | GTTTCG |
| Barcode | SEQ ID | /5Phos/AGGCCAGAGCATTCGAACTCACCGTGGCCGAT |
| A-69 | NO: 69 | GTTTCG |
| Barcode | SEQ ID | /5Phos/AGGCCAGAGCATTCGAAGAGATCGTGGCCGAT |
| A-70 | NO: 70 | GTTTCG |
| Barcode | SEQ ID | /5Phos/AGGCCAGAGCATTCGAAGGACACGTGGCCGAT |
| A-71 | NO: 71 | GTTTCG |
| Barcode | SEQ ID | /5Phos/AGGCCAGAGCATTCGAATCCGTCGTGGCCGAT |
| A-72 | NO: 72 | GTTTCG |
| Barcode | SEQ ID | /5Phos/AGGCCAGAGCATTCGAATGTTGCGTGGCCGAT |
| A-73 | NO: 73 | GTTTCG |
| Barcode | SEQ ID | /5Phos/AGGCCAGAGCATTCGACACGACCGTGGCCGAT |
| A-74 | NO: 74 | GTTTCG |
| Barcode | SEQ ID | /5Phos/AGGCCAGAGCATTCGACAGATTCGTGGCCGAT |
| A-75 | NO: 75 | GTTTCG |
| Barcode | SEQ ID | /5Phos/AGGCCAGAGCATTCGAGATGTACGTGGCCGAT |
| A-76 | NO: 76 | GTTTCG |
| Barcode | SEQ ID | /5Phos/AGGCCAGAGCATTCGAGCACCTCGTGGCCGAT |
| A-77 | NO: 77 | GTTTCG |
| Barcode | SEQ ID | /5Phos/AGGCCAGAGCATTCGAGCCATGCGTGGCCGAT |
| A-78 | NO: 78 | GTTTCG |
| Barcode | SEQ ID | /5Phos/AGGCCAGAGCATTCGAGGCTAACGTGGCCGAT |
| A-79 | NO: 79 | GTTTCG |
| Barcode | SEQ ID | /5Phos/AGGCCAGAGCATTCGATAGCGACGTGGCCGAT |
| A-80 | NO: 80 | GTTTCG |
| Barcode | SEQ ID | /5Phos/AGGCCAGAGCATTCGATCATTCCGTGGCCGAT |
| A-81 | NO: 81 | GTTTCG |
| Barcode | SEQ ID | /5Phos/AGGCCAGAGCATTCGATTGGCTCGTGGCCGAT |
| A-82 | NO: 82 | GTTTCG |
| Barcode | SEQ ID | /5Phos/AGGCCAGAGCATTCGCAAGGAGCGTGGCCGAT |
| A-83 | NO: 83 | GTTTCG |
| Barcode | SEQ ID | /5Phos/AGGCCAGAGCATTCGCACCTTACGTGGCCGAT |
| A-84 | NO: 84 | GTTTCG |
| Barcode | SEQ ID | /5Phos/AGGCCAGAGCATTCGCCATCCTCGTGGCCGAT |
| A-85 | NO: 85 | GTTTCG |
| Barcode | SEQ ID | /5Phos/AGGCCAGAGCATTCGCCGACAACGTGGCCGAT |
| A-86 | NO: 86 | GTTTCG |
| Barcode | SEQ ID | /5Phos/AGGCCAGAGCATTCGCCTAATCCGTGGCCGAT |
| A-87 | NO: 87 | GTTTCG |
| Barcode | SEQ ID | /5Phos/AGGCCAGAGCATTCGCCTCTATCGTGGCCGAT |
| A-88 | NO: 88 | GTTTCG |
| Barcode | SEQ ID | /5Phos/AGGCCAGAGCATTCGCGACACACGTGGCCGAT |
| A-89 | NO: 89 | GTTTCG |
| Barcode | SEQ ID | /5Phos/AGGCCAGAGCATTCGCGGATTGCGTGGCCGAT |
| A-90 | NO: 90 | GTTTCG |
| Barcode | SEQ ID | /5Phos/AGGCCAGAGCATTCGCTAAGGTCGTGGCCGAT |
| A-91 | NO: 91 | GTTTCG |
| Barcode | SEQ ID | /5Phos/AGGCCAGAGCATTCGGAACAGGCGTGGCCGAT |
| A-92 | NO: 92 | GTTTCG |
| Barcode | SEQ ID | /5Phos/AGGCCAGAGCATTCGGACAGTGCGTGGCCGAT |
| A-93 | NO: 93 | GTTTCG |
| Barcode | SEQ ID | /5Phos/AGGCCAGAGCATTCGGAGTTAGCGTGGCCGAT |
| A-94 | NO: 94 | GTTTCG |
| Barcode | SEQ ID | /5Phos/AGGCCAGAGCATTCGGATGAATCGTGGCCGAT |
| A-95 | NO: 95 | GTTTCG |
| Barcode | SEQ ID | /5Phos/AGGCCAGAGCATTCGGCCAAGACGTGGCCGAT |
| A-96 | NO: 96 | GTTTCG |
| Barcode | SEQ ID | /5Phos/AGGCCAGAGCATTCGCGGAAGAAGTGGCCGAT |
| A-97 | NO: 97 | GTTTCG |
| Barcode | SEQ ID | /5Phos/AGGCCAGAGCATTCGGTGACAAGGTGGCCGAT |
| A-98 | NO: 98 | GTTTCG |
| Barcode | SEQ ID | /5Phos/AGGCCAGAGCATTCGGAACCAGAGTGGCCGAT |
| A-99 | NO: 99 | GTTTCG |
| Barcode | SEQ ID | /5Phos/AGGCCAGAGCATTCGTTGCTGGAGTGGCCGAT |
| A-100 | NO: 100 | GTTTCG |

TABLE 5
DNA barcode B sequences.

| Barcode | SEQ ID | |
|---|---|---|
| B | NO: | Sequence |
| Barcode | SEQ ID | CAAGCGTTGGCTTCTCGCATCTAACGTGATATCCACG |
| B-1 | NO: 101 | TGCTTGAG |
| Barcode | SEQ ID | CAAGCGTTGGCTTCTCGCATCTAAACATCGATCCACG |
| B-2 | NO: 102 | TGCTTGAG |
| Barcode | SEQ ID | CAAGCGTTGGCTTCTCGCATCTATGCCTAAATCCACG |
| B-3 | NO: 103 | TGCTTGAG |
| Barcode | SEQ ID | CAAGCGTTGGCTTCTCGCATCTAGTGGTCAATCCACG |
| B-4 | NO: 104 | TGCTTGAG |
| Barcode | SEQ ID | CAAGCGTTGGCTTCTCGCATCTACCACTGTATCCACG |
| B-5 | NO: 105 | TGCTTGAG |
| Barcode | SEQ ID | CAAGCGTTGGCTTCTCGCATCTACATTGGCATCCACG |
| B-6 | NO: 106 | TGCTTGAG |
| Barcode | SEQ ID | CAAGCGTTGGCTTCTCGCATCTCAGATCTGATCCACG |
| B-7 | NO: 107 | TGCTTGAG |
| Barcode | SEQ ID | CAAGCGTTGGCTTCTCGCATCTCATCAAGTATCCACG |
| B-8 | NO: 108 | TGCTTGAG |
| Barcode | SEQ ID | CAAGCGTTGGCTTCTCGCATCTCGCTGATCATCCACG |
| B-9 | NO: 109 | TGCTTGAG |
| Barcode | SEQ ID | CAAGCGTTGGCTTCTCGCATCTACAAGCTAATCCACG |
| B-10 | NO: 110 | TGCTTGAG |
| Barcode | SEQ ID | CAAGCGTTGGCTTCTCGCATCTCTGTAGCCATCCACG |
| B-11 | NO: 111 | TGCTTGAG |
| Barcode | SEQ ID | CAAGCGTTGGCTTCTCGCATCTAGTACAAGATCCACG |
| B-12 | NO: 112 | TGCTTGAG |
| Barcode | SEQ ID | CAAGCGTTGGCTTCTCGCATCTAACAACCAATCCACG |
| B-13 | NO: 113 | TGCTTGAG |
| Barcode | SEQ ID | CAAGCGTTGGCTTCTCGCATCTAACCGAGAATCCACG |
| B-14 | NO: 114 | TGCTTGAG |
| Barcode | SEQ ID | CAAGCGTTGGCTTCTCGCATCTAACGCTTAATCCACG |
| B-15 | NO: 115 | TGCTTGAG |
| Barcode | SEQ ID | CAAGCGTTGGCTTCTCGCATCTAAGACGGAATCCACG |
| B-16 | NO: 116 | TGCTTGAG |
| Barcode | SEQ ID | CAAGCGTTGGCTTCTCGCATCTAAGGTACAATCCACG |
| B-17 | NO: 117 | TGCTTGAG |
| Barcode | SEQ ID | CAAGCGTTGGCTTCTCGCATCTACACAGAAATCCACG |
| B-18 | NO: 118 | TGCTTGAG |
| Barcode | SEQ ID | CAAGCGTTGGCTTCTCGCATCTACAGCAGAATCCACG |
| B-19 | NO: 119 | TGCTTGAG |
| Barcode | SEQ ID | CAAGCGTTGGCTTCTCGCATCTACCTCCAAATCCACG |
| B-20 | NO: 120 | TGCTTGAG |
| Barcode | SEQ ID | CAAGCGTTGGCTTCTCGCATCTACGCTCGAATCCACG |
| B-21 | NO: 121 | TGCTTGAG |
| Barcode | SEQ ID | CAAGCGTTGGCTTCTCGCATCTACGTATCAATCCACG |
| B-22 | NO: 122 | TGCTTGAG |
| Barcode | SEQ ID | CAAGCGTTGGCTTCTCGCATCTACTATGCAATCCACG |
| B-23 | NO: 123 | TGCTTGAG |
| Barcode | SEQ ID | CAAGCGTTGGCTTCTCGCATCTAGAGTCAAATCCACG |
| B-24 | NO: 124 | TGCTTGAG |
| Barcode | SEQ ID | CAAGCGTTGGCTTCTCGCATCTAGATCGCAATCCACG |
| B-25 | NO: 125 | TGCTTGAG |
| Barcode | SEQ ID | CAAGCGTTGGCTTCTCGCATCTAGCAGGAAATCCACG |
| B-26 | NO: 126 | TGCTTGAG |
| Barcode | SEQ ID | CAAGCGTTGGCTTCTCGCATCTAGTCACTAATCCACG |
| B-27 | NO: 127 | TGCTTGAG |
| Barcode | SEQ ID | CAAGCGTTGGCTTCTCGCATCTATCCTGTAATCCACG |
| B-28 | NO: 128 | TGCTTGAG |
| Barcode | SEQ ID | CAAGCGTTGGCTTCTCGCATCTATTGAGGAATCCACG |
| B-29 | NO: 129 | TGCTTGAG |
| Barcode | SEQ ID | CAAGCGTTGGCTTCTCGCATCTCAACCACAATCCACG |
| B-30 | NO: 130 | TGCTTGAG |
| Barcode | SEQ ID | CAAGCGTTGGCTTCTCGCATCTGACTAGTAATCCACG |
| B-31 | NO: 131 | TGCTTGAG |
| Barcode | SEQ ID | CAAGCGTTGGCTTCTCGCATCTCAATGGAAATCCACG |
| B-32 | NO: 132 | TGCTTGAG |
| Barcode | SEQ ID | CAAGCGTTGGCTTCTCGCATCTCACTTCGAATCCACG |
| B-33 | NO: 133 | TGCTTGAG |
| Barcode | SEQ ID | CAAGCGTTGGCTTCTCGCATCTCAGCGTTAATCCACG |
| B-34 | NO: 134 | TGCTTGAG |
| Barcode | SEQ ID | CAAGCGTTGGCTTCTCGCATCTCATACCAAATCCACG |
| B-35 | NO: 135 | TGCTTGAG |
| Barcode | SEQ ID | CAAGCGTTGGCTTCTCGCATCTCCAGTTCAATCCACG |
| B-36 | NO: 136 | TGCTTGAG |
| Barcode | SEQ ID | CAAGCGTTGGCTTCTCGCATCTCCGAAGTAATCCACG |
| B-37 | NO: 137 | TGCTTGAG |
| Barcode | SEQ ID | CAAGCGTTGGCTTCTCGCATCTCCGTGAGAATCCACG |
| B-38 | NO: 138 | TGCTTGAG |
| Barcode | SEQ ID | CAAGCGTTGGCTTCTCGCATCTCCTCCTGAATCCACG |
| B-39 | NO: 139 | TGCTTGAG |
| Barcode | SEQ ID | CAAGCGTTGGCTTCTCGCATCTCGAACTTAATCCACG |
| B-40 | NO: 140 | TGCTTGAG |
| Barcode | SEQ ID | CAAGCGTTGGCTTCTCGCATCTCGACTGGAATCCACG |
| B-41 | NO: 141 | TGCTTGAG |
| Barcode | SEQ ID | CAAGCGTTGGCTTCTCGCATCTCGCATACAATCCACG |
| B-42 | NO: 142 | TGCTTGAG |
| Barcode | SEQ ID | CAAGCGTTGGCTTCTCGCATCTCTCAATGAATCCACG |
| B-43 | NO: 143 | TGCTTGAG |
| Barcode | SEQ ID | CAAGCGTTGGCTTCTCGCATCTCTGAGCCAATCCACG |
| B-44 | NO: 144 | TGCTTGAG |
| Barcode | SEQ ID | CAAGCGTTGGCTTCTCGCATCTCTGGCATAATCCACG |
| B-45 | NO: 145 | TGCTTGAG |
| Barcode | SEQ ID | CAAGCGTTGGCTTCTCGCATCTGAATCTGAATCCACG |
| B-46 | NO: 146 | TGCTTGAG |
| Barcode | SEQ ID | CAAGCGTTGGCTTCTCGCATCTCAAGACTAATCCACG |
| B-47 | NO: 147 | TGCTTGAG |
| Barcode | SEQ ID | CAAGCGTTGGCTTCTCGCATCTGAGCTGAAATCCACG |
| B-48 | NO: 148 | TGCTTGAG |
| Barcode | SEQ ID | CAAGCGTTGGCTTCTCGCATCTGATAGACAATCCACG |
| B-49 | NO: 149 | TGCTTGAG |
| Barcode | SEQ ID | CAAGCGTTGGCTTCTCGCATCTGCCACATAATCCACG |
| B-50 | NO: 150 | TGCTTGAG |
| Barcode | SEQ ID | CAAGCGTTGGCTTCTCGCATCTGCGAGTAAATCCACG |
| B-51 | NO: 151 | TGCTTGAG |
| Barcode | SEQ ID | CAAGCGTTGGCTTCTCGCATCTGCTAACGAATCCACG |
| B-52 | NO: 152 | TGCTTGAG |
| Barcode | SEQ ID | CAAGCGTTGGCTTCTCGCATCTGCTCGGTAATCCACG |
| B-53 | NO: 153 | TGCTTGAG |
| Barcode | SEQ ID | CAAGCGTTGGCTTCTCGCATCTGGAGAACAATCCACG |
| B-54 | NO: 154 | TGCTTGAG |
| Barcode | SEQ ID | CAAGCGTTGGCTTCTCGCATCTGGTGCGAAATCCACG |
| B-55 | NO: 155 | TGCTTGAG |
| Barcode | SEQ ID | CAAGCGTTGGCTTCTCGCATCTGTACGCAAATCCACG |
| B-56 | NO: 156 | TGCTTGAG |
| Barcode | SEQ ID | CAAGCGTTGGCTTCTCGCATCTGTCGTAGAATCCACG |
| B-57 | NO: 157 | TGCTTGAG |
| Barcode | SEQ ID | CAAGCGTTGGCTTCTCGCATCTGTCTGTCAATCCACG |
| B-58 | NO: 158 | TGCTTGAG |
| Barcode | SEQ ID | CAAGCGTTGGCTTCTCGCATCTGTGTTCTAATCCACG |
| B-59 | NO: 159 | TGCTTGAG |
| Barcode | SEQ ID | CAAGCGTTGGCTTCTCGCATCTTAGGATGAATCCACG |
| B-60 | NO: 160 | TGCTTGAG |
| Barcode | SEQ ID | CAAGCGTTGGCTTCTCGCATCTTATCAGCAATCCACG |
| B-61 | NO: 161 | TGCTTGAG |
| Barcode | SEQ ID | CAAGCGTTGGCTTCTCGCATCTTCCGTCTAATCCACG |
| B-62 | NO: 162 | TGCTTGAG |
| Barcode | SEQ ID | CAAGCGTTGGCTTCTCGCATCTTCTTCACAATCCACG |
| B-63 | NO: 163 | TGCTTGAG |
| Barcode | SEQ ID | CAAGCGTTGGCTTCTCGCATCTTGAAGAGAATCCACG |
| B-64 | NO: 164 | TGCTTGAG |
| Barcode | SEQ ID | CAAGCGTTGGCTTCTCGCATCTTGGAACAAATCCACG |
| B-65 | NO: 165 | TGCTTGAG |
| Barcode | SEQ ID | CAAGCGTTGGCTTCTCGCATCTTGGCTTCAATCCACG |
| B-66 | NO: 166 | TGCTTGAG |
| Barcode | SEQ ID | CAAGCGTTGGCTTCTCGCATCTTGGTGGTAATCCACG |
| B-67 | NO: 167 | TGCTTGAG |
| Barcode | SEQ ID | CAAGCGTTGGCTTCTCGCATCTTTCACGCAATCCACG |
| B-68 | NO: 168 | TGCTTGAG |
| Barcode | SEQ ID | CAAGCGTTGGCTTCTCGCATCTAACTCACCATCCACG |
| B-69 | NO: 169 | TGCTTGAG |
| Barcode | SEQ ID | CAAGCGTTGGCTTCTCGCATCTAAGAGATCATCCACG |
| B-70 | NO: 170 | TGCTTGAG |
| Barcode | SEQ ID | CAAGCGTTGGCTTCTCGCATCTAAGGACACATCCACG |
| B-71 | NO: 171 | TGCTTGAG |
| Barcode | SEQ ID | CAAGCGTTGGCTTCTCGCATCTAATCCGTCATCCACG |
| B-72 | NO: 172 | TGCTTGAG |
| Barcode | SEQ ID | CAAGCGTTGGCTTCTCGCATCTAATGTTGCATCCACG |
| B-73 | NO: 173 | TGCTTGAG |
| Barcode | SEQ ID | CAAGCGTTGGCTTCTCGCATCTACACGACCATCCACG |
| B-74 | NO: 174 | TGCTTGAG |
| Barcode | SEQ ID | CAAGCGTTGGCTTCTCGCATCTACAGATTCATCCACG |
| B-75 | NO: 175 | TGCTTGAG |
| Barcode | SEQ ID | CAAGCGTTGGCTTCTCGCATCTAGATGTACATCCACG |
| B-76 | NO: 176 | TGCTTGAG |
| Barcode | SEQ ID | CAAGCGTTGGCTTCTCGCATCTAGCACCTCATCCACG |
| B-77 | NO: 177 | TGCTTGAG |
| Barcode | SEQ ID | CAAGCGTTGGCTTCTCGCATCTAGCCATGCATCCACG |
| B-78 | NO: 178 | TGCTTGAG |
| Barcode | SEQ ID | CAAGCGTTGGCTTCTCGCATCTAGGCTAACATCCACG |
| B-79 | NO: 179 | TGCTTGAG |
| Barcode | SEQ ID | CAAGCGTTGGCTTCTCGCATCTATAGCGACATCCACG |
| B-80 | NO: 180 | TGCTTGAG |
| Barcode | SEQ ID | CAAGCGTTGGCTTCTCGCATCTATCATTCCATCCACG |
| B-81 | NO: 181 | TGCTTGAG |
| Barcode | SEQ ID | CAAGCGTTGGCTTCTCGCATCTATTGGCTCATCCACG |
| B-82 | NO: 182 | TGCTTGAG |
| Barcode | SEQ ID | CAAGCGTTGGCTTCTCGCATCTCAAGGAGCATCCACG |
| B-83 | NO: 183 | TGCTTGAG |
| Barcode | SEQ ID | CAAGCGTTGGCTTCTCGCATCTCACCTTACATCCACG |
| B-84 | NO: 184 | TGCTTGAG |
| Barcode | SEQ ID | CAAGCGTTGGCTTCTCGCATCTCCATCCTCATCCACG |
| B-85 | NO: 185 | TGCTTGAG |
| Barcode | SEQ ID | CAAGCGTTGGCTTCTCGCATCTCCGACAACATCCACG |
| B-86 | NO: 186 | TGCTTGAG |
| Barcode | SEQ ID | CAAGCGTTGGCTTCTCGCATCTCCTAATCCATCCACG |
| B-87 | NO: 187 | TGCTTGAG |
| Barcode | SEQ ID | CAAGCGTTGGCTTCTCGCATCTCCTCTATCATCCACGT |
| B-88 | NO: 188 | GCTTGAG |
| Barcode | SEQ ID | CAAGCGTTGGCTTCTCGCATCTCGACACACATCCACG |
| B-89 | NO: 189 | TGCTTGAG |
| Barcode | SEQ ID | CAAGCGTTGGCTTCTCGCATCTCGGATTGCATCCACG |
| B-90 | NO: 190 | TGCTTGAG |
| Barcode | SEQ ID | CAAGCGTTGGCTTCTCGCATCTCTAAGGTCATCCACG |
| B-91 | NO: 191 | TGCTTGAG |
| Barcode | SEQ ID | CAAGCGTTGGCTTCTCGCATCTGAACAGGCATCCACG |
| B-92 | NO: 192 | TGCTTGAG |
| Barcode | SEQ ID | CAAGCGTTGGCTTCTCGCATCTGACAGTGCATCCACG |
| B-93 | NO: 193 | TGCTTGAG |
| Barcode | SEQ ID | CAAGCGTTGGCTTCTCGCATCTGAGTTAGCATCCACG |
| B-94 | NO: 194 | TGCTTGAG |
| Barcode | SEQ ID | CAAGCGTTGGCTTCTCGCATCTGATGAATCATCCACG |
| B-95 | NO: 195 | TGCTTGAG |
| Barcode | SEQ ID | CAAGCGTTGGCTTCTCGCATCTGCCAAGACATCCACG |
| B-96 | NO: 196 | TGCTTGAG |
| Barcode | SEQ ID | CAAGCGTTGGCTTCTCGCATCTCGGAAGAAATCCACG |
| B-97 | NO: 197 | TGCTTGAG |
| Barcode | SEQ ID | CAAGCGTTGGCTTCTCGCATCTGTGACAAGATCCACG |
| B-98 | NO: 198 | TGCTTGAG |
| Barcode | SEQ ID | CAAGCGTTGGCTTCTCGCATCTGAACCAGAATCCACG |
| B-99 | NO: 199 | TGCTTGAG |
| Barcode | SEQ ID | CAAGCGTTGGCTTCTCGCATCTTTGCTGGAATCCACG |
| B-100 | NO: 200 | TGCTTGAG |

TABLE 6
Chemicals and reagents.

| Name | Catalog number | Vender |
|---|---|---|
| Formaldehyde solution | PI28906 | Thermo Fisher Scientific |
| HEPES pH 7.5 | BBH-75-250 | Boston BioProducts |
| Glycine | 50046 | Sigma-Aldrich |
| NaCl | AM9760G | Thermo Fisher Scientific |
| Digitonin | G9441 | Promega |
| MgCl₂ | AM9530G | Thermo Fisher Scientific |
| Spermidine | S0266 | Sigma-Aldrich |
| EDTA-free Protease Inhibitor | 11873580001 | Millipore Sigma |
| Cocktail | | |
| NP40 | 11332473001 | Sigma-Aldrich |
| EDTA Solution pH 8.0 | AB00502 | AmericanBio |
| Bovine Serum Albumin (BSA) | A8806 | Sigma-Aldrich |
| Anti-H3K27ac antibody | ab177178 | Abcam |
| Anti-H3K27me3 antibody | 9733 | Cell Signaling |
| | | Technology |
| Histone H3K4me3 antibody | 39159 | Active Motif |
| Secondary antibody (Guinea Pig | ABIN101961 | Antibodies-Online |
| anti-Rabbit IgG) | | |
| pA-Tn5 Transposase - unloaded | C01070002 | Diagenode |
| Triton X-100 | T8787 | Sigma-Aldrich |
| T4 DNA Ligase | M0202L | New England Biolabs |
| T4 DNA Ligase Reaction Buffer | B0202S | New England Biolabs |
| NEBuffer 3.1 | B7203S | New England Biolabs |
| DPBS | 14190144 | Thermo Fisher Scientific |
| Proteinase K | EO0491 | Thermo Fisher Scientific |
| Ampure XP beads | A63880 | Beckman Coulter |
| NEBNext High-Fidelity 2X PCR | M0541L | New England Biolabs |
| Master Mix | | |
| SYBR Green I Nucleic Acid Gel | S7563 | Thermo Fisher Scientific |
| Stain | | |
| DNA Clean & Concentrator-5 | D4014 | Zymo Research |
| Tn5 Transposase - unloaded | C01070010 | Diagenode |
| Tagmentation Buffer (2x) | C01019043 | Diagenode |
| Sodium dodecyl sulfate | 71736 | Sigma-Aldrich |
| Maxima H Minus Reverse | EP0751 | Thermo Fisher Scientific |
| Transcriptase (200 U/L) | | |
| dNTP mix | R0192 | Thermo Fisher Scientific |
| SUPERased In RNase Inhibitor | AM2694 | Thermo Fisher Scientific |
| Ampure XP beads | A63880 | Beckman Coulter |
| Dynabeads MyOne C1 | 65001 | Thermo Fisher Scientific |
| RNase Inhibitor | Y9240L | Enzymatics |
| Kapa Hotstart HiFi ReadyMix | KK2601 | Kapa Biosystems |
| Nextera XT DNA Preparation Kit | FC-131-1024 | Illumina |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method, comprising:
(a) delivering to a region of interest in a tissue sample mounted on a substrate reagents for transposition including a Tn5 transposition complex pre-loaded with a DNA adapter containing a universal ligation linker and reagents for reverse transcription including a DNA adapter containing a universal ligation linker and an RNA detection probe;
(b) delivering to the region of interest a first set of barcoded polynucleotides, wherein the barcoded polynucleotides comprise a first region for ligation to the linker adaptor sequence, a second unique region for spatial barcoding and a third linker region for ligation to a region of the second barcode or a universal ligation linker, wherein the first set of barcoded polynucleotides is delivered through a first microfluidic device clamped to the region of interest;
(c) delivering to the region of interest ligation reagents to join the ligation adaptor to the barcoded polynucleotides of the first set;
(d) delivering to the region of interest a second set of barcoded polynucleotides, wherein the barcoded polynucleotides comprise a first region for ligation to the linker region of the first barcode or a universal ligation linker, a second unique region for spatial barcoding and a third ligation region comprising a sequence for recognition by a primer for DNA amplification, wherein the second set of barcoded polynucleotides is delivered through a second microfluidic device clamped to the region of interest, wherein the second microfluidic device is oriented on the region of interest perpendicular to the direction of the microchannels of the first microfluidic device;
(e) delivering to the region of interest ligation reagents to join barcoded polynucleotides of the first set to barcoded polynucleotides of the second set;
(f) imaging the region of interest to produce a sample image;
(g) delivering to the region of interest lysis buffer or denaturation reagents to produce a lysed or denatured tissue sample; and
(h) extracting the cDNA and genomic DNA from the lysed or denatured tissue sample.

2. The method of claim 1, further comprising a step of permeabilizing the tissue sample prior to delivering the transposase and linker adaptor sequence.

3. The method of claim 1, wherein the RNA detection probe comprises a poly-T sequence that binds to the poly-A trail of mRNAs.

4. A method, comprising:
(a) delivering to a region of interest in a tissue sample mounted on a substrate reagents for spatial tagmentation including (i) a primary antibody specific for binding to an epigenomic marker of interest (ii) a secondary antibody and (iii) protein A tethered Tn5-DNA complex pre-loaded with a DNA adapter containing a universal ligation linker, and reagents for reverse transcription including a DNA adapter containing a universal ligation linker and an RNA detection probe;
(b) delivering to the region of interest a first set of barcoded polynucleotides, wherein the barcoded polynucleotides comprise a first region for ligation to the linker adaptor sequence, a second unique region for spatial barcoding and a third linker region for ligation to a region of the second barcode or a universal ligation linker, wherein the first set of barcoded polynucleotides is delivered through a first microfluidic device clamped to the region of interest;
(c) delivering to the region of interest ligation reagents to join the ligation adaptor to the barcoded polynucleotides of the first set;
(d) delivering to the region of interest a second set of barcoded polynucleotides, wherein the barcoded polynucleotides comprise a first region for ligation to the linker region of the first barcode or a universal ligation linker, a second unique region for spatial barcoding and a third ligation region comprising a sequence for recognition by a primer for DNA amplification, wherein the second set of barcoded polynucleotides is delivered through a second microfluidic device clamped to the region of interest, wherein the second microfluidic device is oriented on the region of interest perpendicular to the direction of the microchannels of the first microfluidic device;
(e) delivering to the region of interest ligation reagents to join barcoded polynucleotides of the first set to barcoded polynucleotides of the second set;
(f) imaging the region of interest to produce a sample image;
(g) delivering to the region of interest lysis buffer or denaturation reagents to produce a lysed or denatured tissue sample; and
(h) extracting the cDNA and genomic DNA from the lysed or denatured tissue sample.

5. The method of claim 4, further comprising a step of permeabilizing the tissue sample prior to delivering the transposase and linker adaptor sequence.

6. The method of claim 4, wherein the primary antibody is selected from whole antibodies, Fab antibody fragments, F(ab′)2 antibody fragments, monospecific Fab2 fragments, bispecific Fab2 fragments, trispecific Fab3 fragments, single chain variable fragments (scFvs), bispecific diabodies, trispecific diabodies, scFvFc molecules, nanobodies, and minibodies.

7. The method of claim 4, wherein the epigenomic marker is selected from the group consisting of H2AK5ac, H2AK9ac, H2BK120ac, H2BK12ac, H2BK15ac, H2BK20ac, H2BK5ac, H2Bub, H3, H3ac, H3K14ac, H3K18ac, H3K23ac, H3K23me2, H3K27me1, H3K27me2, H3K36ac, H3K36me1, H3K36me2, H3K4ac, H3K56ac, H3K79me1, H3K79me3, H3K9acS10ph, H3K9me2, H3S10ph, H3T11ph, H4, H4ac, H4K12ac, H4K16ac, H4K5ac, H4K8ac, H4K91ac, H3F3A, H3K27me3, H3K36me3, H3K4me1, H3K79me2, H3K9me1, H3K9me2, H3K9me3, H4K20me1, H2AFZ, H3K27ac, H3K4me2, H3K4me3, and H3K9ac.

8. The method of claim 4, wherein the RNA detection probe comprises a poly-T sequence that binds to the poly-A trail of mRNAs.

9. The method of claim 1, wherein the method further comprises delivering to the biological sample a ligation linker sequence, wherein the ligation linker is selected from the group consisting of:
(a) a nucleic acid molecule comprising a sequence complementary to the ligation linker sequence of the ligation adaptor associated with the transposon and a sequence complementary to the ligation linker sequence of the barcoded polynucleotides of the first set; and
(b) a nucleic acid molecule comprising a sequence complementary to the ligation linker sequence of the barcoded polynucleotides of the first set and a sequence complementary to the ligation linker sequence of the barcoded polynucleotides of the second set.

10. The method of claim 1, further comprising step (i) sequencing the cDNA, the genomic DNA or a combination thereof.

11. The method of claim 10 further comprising constructing at least one of a spatial transcriptomic map and a spatial epigenomic map of the tissue section by matching the spatially addressable barcoded conjugates to corresponding sequencing reads.

12. The method of claim 11 further comprising identifying the anatomical location of the nucleic acids by correlating the spatial map to the sample image.

13. The method of claim 1, wherein the tissue section mounted on a slide is produced by:
sectioning a fixed frozen tissue or a formalin fixed paraffin embedded (FFPE) tissue, optionally into a 5-10 μm section and mounting the tissue section onto a substrate, optionally a poly-L-lysine-coated slide; applying to the tissue section a wash solution, optionally a xylene solution, to deparaffinize the tissue section;
applying to the tissue section a rehydration solution to rehydrate the tissue section;
applying to the tissue section an enzymatic solution to permeabilize the tissue section; and
applying formalin to the tissue section to post-fix the tissue section.

14. The method of any one of claims 1-13, wherein the first and/or second microfluidic device is fabricated from polydimethylsiloxane (PDMS), rubber, plastic, or glass.

15. The method of claim 1, wherein the first and/or second microfluidic device comprises 10 to 1000 microchannels.

16. The method of claim 1, wherein the first and/or second microfluidic device comprises serpentine microchannels.

17. The method of claim 16, further comprising delivering to the region of interest a third set of barcoded polynucleotides, wherein the third set of barcoded polynucleotides is delivered to specific zones, such that each zone distinguishes a specific region of overlap of the first and second barcode sequences; wherein the third set of barcoded polynucleotides are delivered directly to the tissue section, optionally through a set of holes in a device clamped to the substrate, wherein each hole is positioned directly above a zone of overlap of the first and second barcode sequences.

18. The method of claim 1, wherein delivery of the first set of barcoded polynucleotides is delivered through the first microfluidic device using a negative pressure system and/or delivery of the second set of barcoded polynucleotides is delivered through the second microfluidic device using a negative pressure system.

19. The method of claim 1, wherein the lysis buffer or denaturation reagents are delivered directly to the tissue section, optionally through a hole in a device clamped to the substrate, wherein the hole is positioned directly above the region of interest.

20. The method of claim 1, wherein the first set of barcoded polynucleotides comprises SEQ ID NO: 1-100 and/or the second set of barcoded polynucleotides comprises SEQ ID NO: 101-200.

21.-23. (canceled)


SEQUENCE LISTING (216 sequences)

SEQ ID NO: 1 - DNA, 38 bp, synthetic construct
  source 1..38: mol_type = other DNA
aggccagagcattcgaacgtgatgtggccgatgtttcg

SEQ ID NO: 2 - DNA, 38 bp, synthetic construct
  source 1..38: mol_type = other DNA
aggccagagcattcgaaacatcggtggccgatgtttcg

SEQ ID NO: 3 - DNA, 38 bp, synthetic construct
  source 1..38: mol_type = other DNA
aggccagagcattcgatgcctaagtggccgatgtttcg

SEQ ID NO: 4 - DNA, 38 bp, synthetic construct
  source 1..38: mol_type = other DNA
aggccagagcattcgagtggtcagtggccgatgtttcg

SEQ ID NO: 5 - DNA, 38 bp, synthetic construct
  source 1..38: mol_type = other DNA
aggccagagcattcgaccactgtgtggccgatgtttcg

SEQ ID NO: 6 - DNA, 38 bp, synthetic construct
  source 1..38: mol_type = other DNA
aggccagagcattcgacattggcgtggccgatgtttcg

SEQ ID NO: 7 - DNA, 38 bp, synthetic construct
  source 1..38: mol_type = other DNA
aggccagagcattcgcagatctggtggccgatgtttcg

SEQ ID NO: 8 - DNA, 38 bp, synthetic construct
  source 1..38: mol_type = other DNA
aggccagagcattcgcatcaagtgtggccgatgtttcg

SEQ ID NO: 9 - DNA, 38 bp, synthetic construct
  source 1..38: mol_type = other DNA
aggccagagcattcgcgctgatcgtggccgatgtttcg

SEQ ID NO: 10 - DNA, 38 bp, synthetic construct
  source 1..38: mol_type = other DNA
aggccagagcattcgacaagctagtggccgatgtttcg

SEQ ID NO: 11 - DNA, 38 bp, synthetic construct
  source 1..38: mol_type = other DNA
aggccagagcattcgctgtagccgtggccgatgtttcg

SEQ ID NO: 12 - DNA, 38 bp, synthetic construct
  source 1..38: mol_type = other DNA
aggccagagcattcgagtacaaggtggccgatgtttcg

SEQ ID NO: 13 - DNA, 38 bp, synthetic construct
  source 1..38: mol_type = other DNA
aggccagagcattcgaacaaccagtggccgatgtttcg

SEQ ID NO: 14 - DNA, 38 bp, synthetic construct
  source 1..38: mol_type = other DNA
aggccagagcattcgaaccgagagtggccgatgtttcg

SEQ ID NO: 15 - DNA, 38 bp, synthetic construct
  source 1..38: mol_type = other DNA
aggccagagcattcgaacgcttagtggccgatgtttcg

SEQ ID NO: 16 - DNA, 38 bp, synthetic construct
  source 1..38: mol_type = other DNA
aggccagagcattcgaagacggagtggccgatgtttcg

SEQ ID NO: 17 - DNA, 38 bp, synthetic construct
  source 1..38: mol_type = other DNA
aggccagagcattcgaaggtacagtggccgatgtttcg

SEQ ID NO: 18 - DNA, 38 bp, synthetic construct
  source 1..38: mol_type = other DNA
aggccagagcattcgacacagaagtggccgatgtttcg

SEQ ID NO: 19 - DNA, 38 bp, synthetic construct
  source 1..38: mol_type = other DNA
aggccagagcattcgacagcagagtggccgatgtttcg

SEQ ID NO: 20 - DNA, 38 bp, synthetic construct
  source 1..38: mol_type = other DNA
aggccagagcattcgacctccaagtggccgatgtttcg

SEQ ID NO: 21 - DNA, 38 bp, synthetic construct
  source 1..38: mol_type = other DNA
aggccagagcattcgacgctcgagtggccgatgtttcg

SEQ ID NO: 22 - DNA, 38 bp, synthetic construct
  source 1..38: mol_type = other DNA
aggccagagcattcgacgtatcagtggccgatgtttcg

SEQ ID NO: 23 - DNA, 38 bp, synthetic construct
  source 1..38: mol_type = other DNA
aggccagagcattcgactatgcagtggccgatgtttcg

SEQ ID NO: 24 - DNA, 38 bp, synthetic construct
  source 1..38: mol_type = other DNA
aggccagagcattcgagagtcaagtggccgatgtttcg

SEQ ID NO: 25 - DNA, 38 bp, synthetic construct
  source 1..38: mol_type = other DNA
aggccagagcattcgagatcgcagtggccgatgtttcg

SEQ ID NO: 26 - DNA, 38 bp, synthetic construct
  source 1..38: mol_type = other DNA
aggccagagcattcgagcaggaagtggccgatgtttcg

SEQ ID NO: 27 - DNA, 38 bp, synthetic construct
  source 1..38: mol_type = other DNA
aggccagagcattcgagtcactagtggccgatgtttcg

SEQ ID NO: 28 - DNA, 38 bp, synthetic construct
  source 1..38: mol_type = other DNA
aggccagagcattcgatcctgtagtggccgatgtttcg

SEQ ID NO: 29 - DNA, 38 bp, synthetic construct
  source 1..38: mol_type = other DNA
aggccagagcattcgattgaggagtggccgatgtttcg

SEQ ID NO: 30 - DNA, 38 bp, synthetic construct
  source 1..38: mol_type = other DNA
aggccagagcattcgcaaccacagtggccgatgtttcg

SEQ ID NO: 31 - DNA, 38 bp, synthetic construct
  source 1..38: mol_type = other DNA
aggccagagcattcggactagtagtggccgatgtttcg

SEQ ID NO: 32 - DNA, 38 bp, synthetic construct
  source 1..38: mol_type = other DNA
aggccagagcattcgcaatggaagtggccgatgtttcg

SEQ ID NO: 33 - DNA, 38 bp, synthetic construct
  source 1..38: mol_type = other DNA
aggccagagcattcgcacttcgagtggccgatgtttcg

SEQ ID NO: 34 - DNA, 38 bp, synthetic construct
  source 1..38: mol_type = other DNA
aggccagagcattcgcagcgttagtggccgatgtttcg

SEQ ID NO: 35 - DNA, 38 bp, synthetic construct
  source 1..38: mol_type = other DNA
aggccagagcattcgcataccaagtggccgatgtttcg

SEQ ID NO: 36 - DNA, 38 bp, synthetic construct
  source 1..38: mol_type = other DNA
aggccagagcattcgccagttcagtggccgatgtttcg

SEQ ID NO: 37 - DNA, 38 bp, synthetic construct
  source 1..38: mol_type = other DNA
aggccagagcattcgccgaagtagtggccgatgtttcg

SEQ ID NO: 38 - DNA, 38 bp, synthetic construct
  source 1..38: mol_type = other DNA
aggccagagcattcgccgtgagagtggccgatgtttcg

SEQ ID NO: 39 - DNA, 38 bp, synthetic construct
  source 1..38: mol_type = other DNA
aggccagagcattcgcctcctgagtggccgatgtttcg

SEQ ID NO: 40 - DNA, 38 bp, synthetic construct
  source 1..38: mol_type = other DNA
aggccagagcattcgcgaacttagtggccgatgtttcg

SEQ ID NO: 41 - DNA, 38 bp, synthetic construct
  source 1..38: mol_type = other DNA
aggccagagcattcgcgactggagtggccgatgtttcg

SEQ ID NO: 42 - DNA, 38 bp, synthetic construct
  source 1..38: mol_type = other DNA
aggccagagcattcgcgcatacagtggccgatgtttcg

SEQ ID NO: 43 - DNA, 38 bp, synthetic construct
  source 1..38: mol_type = other DNA
aggccagagcattcgctcaatgagtggccgatgtttcg

SEQ ID NO: 44 - DNA, 38 bp, synthetic construct
  source 1..38: mol_type = other DNA
aggccagagcattcgctgagccagtggccgatgtttcg

SEQ ID NO: 45 - DNA, 38 bp, synthetic construct
  source 1..38: mol_type = other DNA
aggccagagcattcgctggcatagtggccgatgtttcg

SEQ ID NO: 46 - DNA, 38 bp, synthetic construct
  source 1..38: mol_type = other DNA
aggccagagcattcggaatctgagtggccgatgtttcg

SEQ ID NO: 47 - DNA, 38 bp, synthetic construct
  source 1..38: mol_type = other DNA
aggccagagcattcgcaagactagtggccgatgtttcg

SEQ ID NO: 48 - DNA, 38 bp, synthetic construct
  source 1..38: mol_type = other DNA
aggccagagcattcggagctgaagtggccgatgtttcg

SEQ ID NO: 49 - DNA, 38 bp, synthetic construct
  source 1..38: mol_type = other DNA
aggccagagcattcggatagacagtggccgatgtttcg

SEQ ID NO: 50 - DNA, 38 bp, synthetic construct
  source 1..38: mol_type = other DNA
aggccagagcattcggccacatagtggccgatgtttcg

SEQ ID NO: 51 - DNA, 38 bp, synthetic construct
  source 1..38: mol_type = other DNA
aggccagagcattcggcgagtaagtggccgatgtttcg

SEQ ID NO: 52 - DNA, 38 bp, synthetic construct
  source 1..38: mol_type = other DNA
aggccagagcattcggctaacgagtggccgatgtttcg

SEQ ID NO: 53 - DNA, 38 bp, synthetic construct
  source 1..38: mol_type = other DNA
aggccagagcattcggctcggtagtggccgatgtttcg

SEQ ID NO: 54 - DNA, 38 bp, synthetic construct
  source 1..38: mol_type = other DNA
aggccagagcattcgggagaacagtggccgatgtttcg

SEQ ID NO: 55 - DNA, 38 bp, synthetic construct
  source 1..38: mol_type = other DNA
aggccagagcattcgggtgcgaagtggccgatgtttcg

SEQ ID NO: 56 - DNA, 38 bp, synthetic construct
  source 1..38: mol_type = other DNA
aggccagagcattcggtacgcaagtggccgatgtttcg

SEQ ID NO: 57 - DNA, 38 bp, synthetic construct
  source 1..38: mol_type = other DNA
aggccagagcattcggtcgtagagtggccgatgtttcg

SEQ ID NO: 58 - DNA, 38 bp, synthetic construct
  source 1..38: mol_type = other DNA
aggccagagcattcggtctgtcagtggccgatgtttcg

SEQ ID NO: 59 - DNA, 38 bp, synthetic construct
  source 1..38: mol_type = other DNA
aggccagagcattcggtgttctagtggccgatgtttcg

SEQ ID NO: 60 - DNA, 38 bp, synthetic construct
  source 1..38: mol_type = other DNA
aggccagagcattcgtaggatgagtggccgatgtttcg

SEQ ID NO: 61 - DNA, 38 bp, synthetic construct
  source 1..38: mol_type = other DNA
aggccagagcattcgtatcagcagtggccgatgtttcg

SEQ ID NO: 62 - DNA, 38 bp, synthetic construct
  source 1..38: mol_type = other DNA
aggccagagcattcgtccgtctagtggccgatgtttcg

SEQ ID NO: 63 - DNA, 38 bp, synthetic construct
  source 1..38: mol_type = other DNA
aggccagagcattcgtcttcacagtggccgatgtttcg

SEQ ID NO: 64 - DNA, 38 bp, synthetic construct
  source 1..38: mol_type = other DNA
aggccagagcattcgtgaagagagtggccgatgtttcg

SEQ ID NO: 65 - DNA, 38 bp, synthetic construct
  source 1..38: mol_type = other DNA
aggccagagcattcgtggaacaagtggccgatgtttcg

SEQ ID NO: 66 - DNA, 38 bp, synthetic construct
  source 1..38: mol_type = other DNA
aggccagagcattcgtggcttcagtggccgatgtttcg

SEQ ID NO: 67 - DNA, 38 bp, synthetic construct
  source 1..38: mol_type = other DNA
aggccagagcattcgtggtggtagtggccgatgtttcg

SEQ ID NO: 68 - DNA, 38 bp, synthetic construct
  source 1..38: mol_type = other DNA
aggccagagcattcgttcacgcagtggccgatgtttcg

SEQ ID NO: 69 - DNA, 38 bp, synthetic construct
  source 1..38: mol_type = other DNA
aggccagagcattcgaactcaccgtggccgatgtttcg

SEQ ID NO: 70 - DNA, 38 bp, synthetic construct
  source 1..38: mol_type = other DNA
aggccagagcattcgaagagatcgtggccgatgtttcg

SEQ ID NO: 71 - DNA, 38 bp, synthetic construct
  source 1..38: mol_type = other DNA
aggccagagcattcgaaggacacgtggccgatgtttcg

SEQ ID NO: 72 - DNA, 38 bp, synthetic construct
  source 1..38: mol_type = other DNA
aggccagagcattcgaatccgtcgtggccgatgtttcg

SEQ ID NO: 73 - DNA, 38 bp, synthetic construct
  source 1..38: mol_type = other DNA
aggccagagcattcgaatgttgcgtggccgatgtttcg

SEQ ID NO: 74 - DNA, 38 bp, synthetic construct
  source 1..38: mol_type = other DNA
aggccagagcattcgacacgaccgtggccgatgtttcg

SEQ ID NO: 75 - DNA, 38 bp, synthetic construct
  source 1..38: mol_type = other DNA
aggccagagcattcgacagattcgtggccgatgtttcg

SEQ ID NO: 76 - DNA, 38 bp, synthetic construct
  source 1..38: mol_type = other DNA
aggccagagcattcgagatgtacgtggccgatgtttcg

SEQ ID NO: 77 - DNA, 38 bp, synthetic construct
  source 1..38: mol_type = other DNA
aggccagagcattcgagcacctcgtggccgatgtttcg

SEQ ID NO: 78 - DNA, 38 bp, synthetic construct
  source 1..38: mol_type = other DNA
aggccagagcattcgagccatgcgtggccgatgtttcg

SEQ ID NO: 79 - DNA, 38 bp, synthetic construct
  source 1..38: mol_type = other DNA
aggccagagcattcgaggctaacgtggccgatgtttcg

SEQ ID NO: 80 - DNA, 38 bp, synthetic construct
  source 1..38: mol_type = other DNA
aggccagagcattcgatagcgacgtggccgatgtttcg

SEQ ID NO: 81 - DNA, 38 bp, synthetic construct
  source 1..38: mol_type = other DNA
aggccagagcattcgatcattccgtggccgatgtttcg

SEQ ID NO: 82 - DNA, 38 bp, synthetic construct
  source 1..38: mol_type = other DNA
aggccagagcattcgattggctcgtggccgatgtttcg

SEQ ID NO: 83 - DNA, 38 bp, synthetic construct
  source 1..38: mol_type = other DNA
aggccagagcattcgcaaggagcgtggccgatgtttcg

SEQ ID NO: 84 - DNA, 38 bp, synthetic construct
  source 1..38: mol_type = other DNA
aggccagagcattcgcaccttacgtggccgatgtttcg

SEQ ID NO: 85 - DNA, 38 bp, synthetic construct
  source 1..38: mol_type = other DNA
aggccagagcattcgccatcctcgtggccgatgtttcg

SEQ ID NO: 86 - DNA, 38 bp, synthetic construct
  source 1..38: mol_type = other DNA
aggccagagcattcgccgacaacgtggccgatgtttcg

SEQ ID NO: 87 - DNA, 38 bp, synthetic construct
  source 1..38: mol_type = other DNA
aggccagagcattcgcctaatccgtggccgatgtttcg

SEQ ID NO: 88 - DNA, 38 bp, synthetic construct
  source 1..38: mol_type = other DNA
aggccagagcattcgcctctatcgtggccgatgtttcg

SEQ ID NO: 89 - DNA, 38 bp, synthetic construct
  source 1..38: mol_type = other DNA
aggccagagcattcgcgacacacgtggccgatgtttcg

SEQ ID NO: 90 - DNA, 38 bp, synthetic construct
  source 1..38: mol_type = other DNA
aggccagagcattcgcggattgcgtggccgatgtttcg

SEQ ID NO: 91 - DNA, 38 bp, synthetic construct
  source 1..38: mol_type = other DNA
aggccagagcattcgctaaggtcgtggccgatgtttcg

SEQ ID NO: 92 - DNA, 38 bp, synthetic construct
  source 1..38: mol_type = other DNA
aggccagagcattcggaacaggcgtggccgatgtttcg

SEQ ID NO: 93 - DNA, 38 bp, synthetic construct
  source 1..38: mol_type = other DNA
aggccagagcattcggacagtgcgtggccgatgtttcg

SEQ ID NO: 94 - DNA, 38 bp, synthetic construct
  source 1..38: mol_type = other DNA
aggccagagcattcggagttagcgtggccgatgtttcg

SEQ ID NO: 95 - DNA, 38 bp, synthetic construct
  source 1..38: mol_type = other DNA
aggccagagcattcggatgaatcgtggccgatgtttcg

SEQ ID NO: 96 - DNA, 38 bp, synthetic construct
  source 1..38: mol_type = other DNA
aggccagagcattcggccaagacgtggccgatgtttcg

SEQ ID NO: 97 - DNA, 38 bp, synthetic construct
  source 1..38: mol_type = other DNA
aggccagagcattcgcggaagaagtggccgatgtttcg

SEQ ID NO: 98 - DNA, 38 bp, synthetic construct
  source 1..38: mol_type = other DNA
aggccagagcattcggtgacaaggtggccgatgtttcg

SEQ ID NO: 99 - DNA, 38 bp, synthetic construct
  source 1..38: mol_type = other DNA
aggccagagcattcggaaccagagtggccgatgtttcg

SEQ ID NO: 100 - DNA, 38 bp, synthetic construct
  source 1..38: mol_type = other DNA
aggccagagcattcgttgctggagtggccgatgtttcg

SEQ ID NO: 101 - DNA, 45 bp, synthetic construct
  source 1..45: mol_type = other DNA
caagcgttggcttctcgcatctaacgtgatatccacgtgcttgag

SEQ ID NO: 102 - DNA, 45 bp, synthetic construct
  source 1..45: mol_type = other DNA
caagcgttggcttctcgcatctaaacatcgatccacgtgcttgag

SEQ ID NO: 103 - DNA, 45 bp, synthetic construct
  source 1..45: mol_type = other DNA
caagcgttggcttctcgcatctatgcctaaatccacgtgcttgag

SEQ ID NO: 104 - DNA, 45 bp, synthetic construct
  source 1..45: mol_type = other DNA
caagcgttggcttctcgcatctagtggtcaatccacgtgcttgag

SEQ ID NO: 105 - DNA, 45 bp, synthetic construct
  source 1..45: mol_type = other DNA
caagcgttggcttctcgcatctaccactgtatccacgtgcttgag

SEQ ID NO: 106 - DNA, 45 bp, synthetic construct
  source 1..45: mol_type = other DNA
caagcgttggcttctcgcatctacattggcatccacgtgcttgag

SEQ ID NO: 107 - DNA, 45 bp, synthetic construct
  source 1..45: mol_type = other DNA
caagcgttggcttctcgcatctcagatctgatccacgtgcttgag

SEQ ID NO: 108 - DNA, 45 bp, synthetic construct
  source 1..45: mol_type = other DNA
caagcgttggcttctcgcatctcatcaagtatccacgtgcttgag

SEQ ID NO: 109 - DNA, 45 bp, synthetic construct
  source 1..45: mol_type = other DNA
caagcgttggcttctcgcatctcgctgatcatccacgtgcttgag

SEQ ID NO: 110 - DNA, 45 bp, synthetic construct
  source 1..45: mol_type = other DNA
caagcgttggcttctcgcatctacaagctaatccacgtgcttgag

SEQ ID NO: 111 - DNA, 45 bp, synthetic construct
  source 1..45: mol_type = other DNA
caagcgttggcttctcgcatctctgtagccatccacgtgcttgag

SEQ ID NO: 112 - DNA, 45 bp, synthetic construct
  source 1..45: mol_type = other DNA
caagcgttggcttctcgcatctagtacaagatccacgtgcttgag

SEQ ID NO: 113 - DNA, 45 bp, synthetic construct
  source 1..45: mol_type = other DNA
caagcgttggcttctcgcatctaacaaccaatccacgtgcttgag

SEQ ID NO: 114 - DNA, 45 bp, synthetic construct
  source 1..45: mol_type = other DNA
caagcgttggcttctcgcatctaaccgagaatccacgtgcttgag

SEQ ID NO: 115 - DNA, 45 bp, synthetic construct
  source 1..45: mol_type = other DNA
caagcgttggcttctcgcatctaacgcttaatccacgtgcttgag

SEQ ID NO: 116 - DNA, 45 bp, synthetic construct
  source 1..45: mol_type = other DNA
caagcgttggcttctcgcatctaagacggaatccacgtgcttgag

SEQ ID NO: 117 - DNA, 45 bp, synthetic construct
  source 1..45: mol_type = other DNA
caagcgttggcttctcgcatctaaggtacaatccacgtgcttgag

SEQ ID NO: 118 - DNA, 45 bp, synthetic construct
  source 1..45: mol_type = other DNA
caagcgttggcttctcgcatctacacagaaatccacgtgcttgag

SEQ ID NO: 119 - DNA, 45 bp, synthetic construct
  source 1..45: mol_type = other DNA
caagcgttggcttctcgcatctacagcagaatccacgtgcttgag

SEQ ID NO: 120 - DNA, 45 bp, synthetic construct
  source 1..45: mol_type = other DNA
caagcgttggcttctcgcatctacctccaaatccacgtgcttgag

SEQ ID NO: 121 - DNA, 45 bp, synthetic construct
  source 1..45: mol_type = other DNA
caagcgttggcttctcgcatctacgctcgaatccacgtgcttgag

SEQ ID NO: 122 - DNA, 45 bp, synthetic construct
  source 1..45: mol_type = other DNA
caagcgttggcttctcgcatctacgtatcaatccacgtgcttgag

SEQ ID NO: 123 - DNA, 45 bp, synthetic construct
  source 1..45: mol_type = other DNA
caagcgttggcttctcgcatctactatgcaatccacgtgcttgag

SEQ ID NO: 124 - DNA, 45 bp, synthetic construct
  source 1..45: mol_type = other DNA
caagcgttggcttctcgcatctagagtcaaatccacgtgcttgag

SEQ ID NO: 125 - DNA, 45 bp, synthetic construct
  source 1..45: mol_type = other DNA
caagcgttggcttctcgcatctagatcgcaatccacgtgcttgag

SEQ ID NO: 126 - DNA, 45 bp, synthetic construct
  source 1..45: mol_type = other DNA
caagcgttggcttctcgcatctagcaggaaatccacgtgcttgag

SEQ ID NO: 127 - DNA, 45 bp, synthetic construct
  source 1..45: mol_type = other DNA
caagcgttggcttctcgcatctagtcactaatccacgtgcttgag

SEQ ID NO: 128 - DNA, 45 bp, synthetic construct
  source 1..45: mol_type = other DNA
caagcgttggcttctcgcatctatcctgtaatccacgtgcttgag

SEQ ID NO: 129 - DNA, 45 bp, synthetic construct
  source 1..45: mol_type = other DNA
caagcgttggcttctcgcatctattgaggaatccacgtgcttgag

SEQ ID NO: 130 - DNA, 45 bp, synthetic construct
  source 1..45: mol_type = other DNA
caagcgttggcttctcgcatctcaaccacaatccacgtgcttgag

SEQ ID NO: 131 - DNA, 45 bp, synthetic construct
  source 1..45: mol_type = other DNA
caagcgttggcttctcgcatctgactagtaatccacgtgcttgag

SEQ ID NO: 132 - DNA, 45 bp, synthetic construct
  source 1..45: mol_type = other DNA
caagcgttggcttctcgcatctcaatggaaatccacgtgcttgag

SEQ ID NO: 133 - DNA, 45 bp, synthetic construct
  source 1..45: mol_type = other DNA
caagcgttggcttctcgcatctcacttcgaatccacgtgcttgag

SEQ ID NO: 134 - DNA, 45 bp, synthetic construct
  source 1..45: mol_type = other DNA
caagcgttggcttctcgcatctcagcgttaatccacgtgcttgag

SEQ ID NO: 135 - DNA, 45 bp, synthetic construct
  source 1..45: mol_type = other DNA
caagcgttggcttctcgcatctcataccaaatccacgtgcttgag

SEQ ID NO: 136 - DNA, 45 bp, synthetic construct
  source 1..45: mol_type = other DNA
caagcgttggcttctcgcatctccagttcaatccacgtgcttgag

SEQ ID NO: 137 - DNA, 45 bp, synthetic construct
  source 1..45: mol_type = other DNA
caagcgttggcttctcgcatctccgaagtaatccacgtgcttgag

SEQ ID NO: 138 - DNA, 45 bp, synthetic construct
  source 1..45: mol_type = other DNA
caagcgttggcttctcgcatctccgtgagaatccacgtgcttgag

SEQ ID NO: 139 - DNA, 45 bp, synthetic construct
  source 1..45: mol_type = other DNA
caagcgttggcttctcgcatctcctcctgaatccacgtgcttgag

SEQ ID NO: 140 - DNA, 45 bp, synthetic construct
  source 1..45: mol_type = other DNA
caagcgttggcttctcgcatctcgaacttaatccacgtgcttgag

SEQ ID NO: 141 - DNA, 45 bp, synthetic construct
  source 1..45: mol_type = other DNA
caagcgttggcttctcgcatctcgactggaatccacgtgcttgag

SEQ ID NO: 142 - DNA, 45 bp, synthetic construct
  source 1..45: mol_type = other DNA
caagcgttggcttctcgcatctcgcatacaatccacgtgcttgag

SEQ ID NO: 143 - DNA, 45 bp, synthetic construct
  source 1..45: mol_type = other DNA
caagcgttggcttctcgcatctctcaatgaatccacgtgcttgag

SEQ ID NO: 144 - DNA, 45 bp, synthetic construct
  source 1..45: mol_type = other DNA
caagcgttggcttctcgcatctctgagccaatccacgtgcttgag

SEQ ID NO: 145 - DNA, 45 bp, synthetic construct
  source 1..45: mol_type = other DNA
caagcgttggcttctcgcatctctggcataatccacgtgcttgag

SEQ ID NO: 146 - DNA, 45 bp, synthetic construct
  source 1..45: mol_type = other DNA
caagcgttggcttctcgcatctgaatctgaatccacgtgcttgag

SEQ ID NO: 147 - DNA, 45 bp, synthetic construct
  source 1..45: mol_type = other DNA
caagcgttggcttctcgcatctcaagactaatccacgtgcttgag

SEQ ID NO: 148 - DNA, 45 bp, synthetic construct
  source 1..45: mol_type = other DNA
caagcgttggcttctcgcatctgagctgaaatccacgtgcttgag

SEQ ID NO: 149 - DNA, 45 bp, synthetic construct
  source 1..45: mol_type = other DNA
caagcgttggcttctcgcatctgatagacaatccacgtgcttgag

SEQ ID NO: 150 - DNA, 45 bp, synthetic construct
  source 1..45: mol_type = other DNA
caagcgttggcttctcgcatctgccacataatccacgtgcttgag

SEQ ID NO: 151 - DNA, 45 bp, synthetic construct
  source 1..45: mol_type = other DNA
caagcgttggcttctcgcatctgcgagtaaatccacgtgcttgag

SEQ ID NO: 152 - DNA, 45 bp, synthetic construct
  source 1..45: mol_type = other DNA
caagcgttggcttctcgcatctgctaacgaatccacgtgcttgag

SEQ ID NO: 153 - DNA, 45 bp, synthetic construct
  source 1..45: mol_type = other DNA
caagcgttggcttctcgcatctgctcggtaatccacgtgcttgag

SEQ ID NO: 154 - DNA, 45 bp, synthetic construct
  source 1..45: mol_type = other DNA
caagcgttggcttctcgcatctggagaacaatccacgtgcttgag

SEQ ID NO: 155 - DNA, 45 bp, synthetic construct
  source 1..45: mol_type = other DNA
caagcgttggcttctcgcatctggtgcgaaatccacgtgcttgag

SEQ ID NO: 156 - DNA, 45 bp, synthetic construct
  source 1..45: mol_type = other DNA
caagcgttggcttctcgcatctgtacgcaaatccacgtgcttgag

SEQ ID NO: 157 - DNA, 45 bp, synthetic construct
  source 1..45: mol_type = other DNA
caagcgttggcttctcgcatctgtcgtagaatccacgtgcttgag

SEQ ID NO: 158 - DNA, 45 bp, synthetic construct
  source 1..45: mol_type = other DNA
caagcgttggcttctcgcatctgtctgtcaatccacgtgcttgag

SEQ ID NO: 159 - DNA, 45 bp, synthetic construct
  source 1..45: mol_type = other DNA
caagcgttggcttctcgcatctgtgttctaatccacgtgcttgag

SEQ ID NO: 160 - DNA, 45 bp, synthetic construct
  source 1..45: mol_type = other DNA
caagcgttggcttctcgcatcttaggatgaatccacgtgcttgag

SEQ ID NO: 161 - DNA, 45 bp, synthetic construct
  source 1..45: mol_type = other DNA
caagcgttggcttctcgcatcttatcagcaatccacgtgcttgag

SEQ ID NO: 162 - DNA, 45 bp, synthetic construct
  source 1..45: mol_type = other DNA
caagcgttggcttctcgcatcttccgtctaatccacgtgcttgag

SEQ ID NO: 163 - DNA, 45 bp, synthetic construct
  source 1..45: mol_type = other DNA
caagcgttggcttctcgcatcttcttcacaatccacgtgcttgag

SEQ ID NO: 164 - DNA, 45 bp, synthetic construct
  source 1..45: mol_type = other DNA
caagcgttggcttctcgcatcttgaagagaatccacgtgcttgag

SEQ ID NO: 165 - DNA, 45 bp, synthetic construct
  source 1..45: mol_type = other DNA
caagcgttggcttctcgcatcttggaacaaatccacgtgcttgag

SEQ ID NO: 166 - DNA, 45 bp, synthetic construct
  source 1..45: mol_type = other DNA
caagcgttggcttctcgcatcttggcttcaatccacgtgcttgag

SEQ ID NO: 167 - DNA, 45 bp, synthetic construct
  source 1..45: mol_type = other DNA
caagcgttggcttctcgcatcttggtggtaatccacgtgcttgag

SEQ ID NO: 168 - DNA, 45 bp, synthetic construct
  source 1..45: mol_type = other DNA
caagcgttggcttctcgcatctttcacgcaatccacgtgcttgag

SEQ ID NO: 169 - DNA, 45 bp, synthetic construct
  source 1..45: mol_type = other DNA
caagcgttggcttctcgcatctaactcaccatccacgtgcttgag

SEQ ID NO: 170 - DNA, 45 bp, synthetic construct
  source 1..45: mol_type = other DNA
caagcgttggcttctcgcatctaagagatcatccacgtgcttgag

SEQ ID NO: 171 - DNA, 45 bp, synthetic construct
  source 1..45: mol_type = other DNA
caagcgttggcttctcgcatctaaggacacatccacgtgcttgag

SEQ ID NO: 172 - DNA, 45 bp, synthetic construct
  source 1..45: mol_type = other DNA
caagcgttggcttctcgcatctaatccgtcatccacgtgcttgag

SEQ ID NO: 173 - DNA, 45 bp, synthetic construct
  source 1..45: mol_type = other DNA
caagcgttggcttctcgcatctaatgttgcatccacgtgcttgag

SEQ ID NO: 174 - DNA, 45 bp, synthetic construct
  source 1..45: mol_type = other DNA
caagcgttggcttctcgcatctacacgaccatccacgtgcttgag

SEQ ID NO: 175 - DNA, 45 bp, synthetic construct
  source 1..45: mol_type = other DNA
caagcgttggcttctcgcatctacagattcatccacgtgcttgag

SEQ ID NO: 176 - DNA, 45 bp, synthetic construct
  source 1..45: mol_type = other DNA
caagcgttggcttctcgcatctagatgtacatccacgtgcttgag

SEQ ID NO: 177 - DNA, 45 bp, synthetic construct
  source 1..45: mol_type = other DNA
caagcgttggcttctcgcatctagcacctcatccacgtgcttgag

SEQ ID NO: 178 - DNA, 45 bp, synthetic construct
  source 1..45: mol_type = other DNA
caagcgttggcttctcgcatctagccatgcatccacgtgcttgag

SEQ ID NO: 179 - DNA, 45 bp, synthetic construct
  source 1..45: mol_type = other DNA
caagcgttggcttctcgcatctaggctaacatccacgtgcttgag

SEQ ID NO: 180 - DNA, 45 bp, synthetic construct
  source 1..45: mol_type = other DNA
caagcgttggcttctcgcatctatagcgacatccacgtgcttgag

SEQ ID NO: 181 - DNA, 45 bp, synthetic construct
  source 1..45: mol_type = other DNA
caagcgttggcttctcgcatctatcattccatccacgtgcttgag

SEQ ID NO: 182 - DNA, 45 bp, synthetic construct
  source 1..45: mol_type = other DNA
caagcgttggcttctcgcatctattggctcatccacgtgcttgag

SEQ ID NO: 183 - DNA, 45 bp, synthetic construct
  source 1..45: mol_type = other DNA
caagcgttggcttctcgcatctcaaggagcatccacgtgcttgag

SEQ ID NO: 184 - DNA, 45 bp, synthetic construct
  source 1..45: mol_type = other DNA
caagcgttggcttctcgcatctcaccttacatccacgtgcttgag

SEQ ID NO: 185 - DNA, 45 bp, synthetic construct
  source 1..45: mol_type = other DNA
caagcgttggcttctcgcatctccatcctcatccacgtgcttgag

SEQ ID NO: 186 - DNA, 45 bp, synthetic construct
  source 1..45: mol_type = other DNA
caagcgttggcttctcgcatctccgacaacatccacgtgcttgag

SEQ ID NO: 187 - DNA, 45 bp, synthetic construct
  source 1..45: mol_type = other DNA
caagcgttggcttctcgcatctcctaatccatccacgtgcttgag

SEQ ID NO: 188 - DNA, 45 bp, synthetic construct
  source 1..45: mol_type = other DNA
caagcgttggcttctcgcatctcctctatcatccacgtgcttgag

SEQ ID NO: 189 - DNA, 45 bp, synthetic construct
  source 1..45: mol_type = other DNA
caagcgttggcttctcgcatctcgacacacatccacgtgcttgag

SEQ ID NO: 190 - DNA, 45 bp, synthetic construct
  source 1..45: mol_type = other DNA
caagcgttggcttctcgcatctcggattgcatccacgtgcttgag

SEQ ID NO: 191 - DNA, 45 bp, synthetic construct
  source 1..45: mol_type = other DNA
caagcgttggcttctcgcatctctaaggtcatccacgtgcttgag

SEQ ID NO: 192 - DNA, 45 bp, synthetic construct
  source 1..45: mol_type = other DNA
caagcgttggcttctcgcatctgaacaggcatccacgtgcttgag

SEQ ID NO: 193 - DNA, 45 bp, synthetic construct
  source 1..45: mol_type = other DNA
caagcgttggcttctcgcatctgacagtgcatccacgtgcttgag

SEQ ID NO: 194 - DNA, 45 bp, synthetic construct
  source 1..45: mol_type = other DNA
caagcgttggcttctcgcatctgagttagcatccacgtgcttgag

SEQ ID NO: 195 - DNA, 45 bp, synthetic construct
  source 1..45: mol_type = other DNA
caagcgttggcttctcgcatctgatgaatcatccacgtgcttgag

SEQ ID NO: 196 - DNA, 45 bp, synthetic construct
  source 1..45: mol_type = other DNA
caagcgttggcttctcgcatctgccaagacatccacgtgcttgag

SEQ ID NO: 197 - DNA, 45 bp, synthetic construct
  source 1..45: mol_type = other DNA
caagcgttggcttctcgcatctcggaagaaatccacgtgcttgag

SEQ ID NO: 198 - DNA, 45 bp, synthetic construct
  source 1..45: mol_type = other DNA
caagcgttggcttctcgcatctgtgacaagatccacgtgcttgag

SEQ ID NO: 199 - DNA, 45 bp, synthetic construct
  source 1..45: mol_type = other DNA
caagcgttggcttctcgcatctgaaccagaatccacgtgcttgag

SEQ ID NO: 200 - DNA, 45 bp, synthetic construct
  source 1..45: mol_type = other DNA
caagcgttggcttctcgcatctttgctggaatccacgtgcttgag

SEQ ID NO: 201 - DNA, 43 bp, synthetic construct
  source 1..43: mol_type = other DNA
  modified_base 26: mod_base = OTHER; note = iBiodT - biotinylated base
catcggcgtacgactnnnnnnnnnnttttttttttttttttvn

SEQ ID NO: 202 - DNA, 30 bp, synthetic construct
  source 1..30: mol_type = other DNA
agtcgtacgccgatgcgaaacatcggccac

SEQ ID NO: 203 - DNA, 30 bp, synthetic construct
  source 1..30: mol_type = other DNA
cgaatgctctggcctctcaagcacgtggat

SEQ ID NO: 204 - DNA, 22 bp, synthetic construct
  source 1..22: mol_type = other DNA
caagcgttggcttctcgcatct

SEQ ID NO: 205 - DNA, 23 bp, synthetic construct
  source 1..23: mol_type = other DNA
aagcagtggtatcaacgcagagt

SEQ ID NO: 206 - DNA, 70 bp, synthetic construct
  source 1..70: mol_type = other DNA
aatgatacggcgaccaccgagatctacactagatcgctcgtcggcagcgtcagatgtgtataagagacag

SEQ ID NO: 207 - DNA, 88 bp, synthetic construct
  source 1..88: mol_type = other DNA
caagcagaagacggcatacgagattcgccttagtctcgtgggctcggagatgtgtataagagacagcaagcgttggcttctcgcatct

SEQ ID NO: 208 - DNA, 88 bp, synthetic construct
  source 1..88: mol_type = other DNA
caagcagaagacggcatacgagatctagtacggtctcgtgggctcggagatgtgtataagagacagcaagcgttggcttctcgcatct

SEQ ID NO: 209 - DNA, 88 bp, synthetic construct
  source 1..88: mol_type = other DNA
caagcagaagacggcatacgagatttctgcctgtctcgtgggctcggagatgtgtataagagacagcaagcgttggcttctcgcatct

SEQ ID NO: 210 - DNA, 88 bp, synthetic construct
  source 1..88: mol_type = other DNA
caagcagaagacggcatacgagatgctcaggagtctcgtgggctcggagatgtgtataagagacagcaagcgttggcttctcgcatct

SEQ ID NO: 211 - DNA, 88 bp, synthetic construct
  source 1..88: mol_type = other DNA
caagcagaagacggcatacgagataggagtccgtctcgtgggctcggagatgtgtataagagacagcaagcgttggcttctcgcatct

SEQ ID NO: 212 - DNA, 88 bp, synthetic construct
  source 1..88: mol_type = other DNA
caagcagaagacggcatacgagatcatgcctagtctcgtgggctcggagatgtgtataagagacagcaagcgttggcttctcgcatct

SEQ ID NO: 213 - DNA, 88 bp, synthetic construct
  source 1..88: mol_type = other DNA
caagcagaagacggcatacgagatgtagagaggtctcgtgggctcggagatgtgtataagagacagcaagcgttggcttctcgcatct

SEQ ID NO: 214 - DNA, 33 bp, synthetic construct
  source 1..33: mol_type = other DNA
tcgtcggcagcgtcagatgtgtataagagacag

SEQ ID NO: 215 - DNA, 19 bp, synthetic construct
  source 1..19: mol_type = other DNA
ctgtctcttatacacatct

SEQ ID NO: 216 - DNA, 34 bp, synthetic construct
  source 1..34: mol_type = other DNA
catcggcgtacgactagatgtgtataagagacag